(19) United States
(12) Patent Application Publication
(10) Pub. No.: US 2017/0050962 A1
(43) Pub. Date: Feb. 23, 2017

(54) IMIDAZO[1,2-A]PYRIDINES AS STIMULATORS OF SOLUBLE GUANYLATE CYCLASE FOR TREATING CARDIOVASCULAR DISEASES

(71) Applicant: BAYER PHARMA AKTIENGESELLSCHAFT, Berlin (DE)

(72) Inventors: Alexandros VAKALOPOULOS, Hilden (DE); Markus FOLLMANN, Köln (DE); Frank WUNDER, Wuppertal (DE); Johannes-Peter STASCH, Grottaferrata (RM) (IT); Tobias MARQUARDT, Wuppertal (DE); Lisa DIETZ, Wuppertal (DE); Volkhart Min-Jian LI, Velbert (DE); Nicholas Charles RAY, Essex (GB); Ines VUJASINOVI&#262;, Zagreb (HR)

(73) Assignee: BAYER PHARMA AKTIENGESELLSCHAFT, Berlin (DE)

(21) Appl. No.: 15/308,230

(22) PCT Filed: Apr. 29, 2015
(86) PCT No.: PCT/EP2015/059275

§ 371 Date: Nov. 1, 2016

(30) Foreign Application Priority Data
May 2, 2014 (EP) 14166910.1

(51) Int. Cl. C07D 471/04; A61K 31/444; A61K 31/437; A61K 45/06

(52) U.S. Cl. CPC C07D 471/04; A61K 45/06; A61K 31/444; A61K 31/437

(57) ABSTRACT

The present application relates to novel heterocyclyl- and heteroaryl-substituted imidazo[1,2-a]pyridines, to processes for preparation thereof, to the use thereof, alone or in combinations, for the treatment and/or prophylaxis of diseases, and to the use thereof for production of medicaments for the treatment and/or prophylaxis of diseases, especially for the treatment and/or prophylaxis of cardiovascular disorders.

The present application relates to novel heterocyclyl- and heteroaryl-substituted imidazo[1,2-a]pyridines, to processes for preparation thereof, to the use thereof, alone or in combinations, for the treatment and/or prophylaxis of diseases, and to the use thereof for production of medicaments for the treatment and/or prophylaxis of diseases, especially for the treatment and/or prophylaxis of cardiovascular disorders.

One of the most important cellular transmission systems in mammalian cells is cyclic guanosine monophosphate (cGMP). Together with nitrogen monoxide (NO), which is released from the endothelium and transmits hormonal and mechanical signals, it forms the NO/cGMP system. Guanylate cyclases catalyse the biosynthesis of cGMP from guanosine triphosphate (GTP). The representatives of this family known to date can be classified into two groups either by structural features or by the type of ligands: the particulate guanylate cyclases which can be stimulated by natriuretic peptides, and the soluble guanylate cyclases which can be stimulated by NO. The soluble guanylate cyclases consist of two subunits and very probably contain one haem per heterodimer, which is part of the regulatory centre. This is of central importance for the activation mechanism. NO is able to bind to the iron atom of haem and thus markedly increase the activity of the enzyme. Haem-free preparations cannot, by contrast, be stimulated by NO. Carbon monoxide (CO) is also able to bind to the central iron atom of haem, but the stimulation by CO is much less than that by NO.

By forming cGMP, and owing to the resulting regulation of phosphodiesterases, ion channels and protein kinases, guanylate cyclase plays an important role in various physiological processes, in particular in the relaxation and proliferation of smooth muscle cells, in platelet aggregation and platelet adhesion and in neuronal signal transmission, and also in disorders which are based on a disruption of the aforementioned processes. Under pathophysiological conditions, the NO/cGMP system can be suppressed, which can lead, for example, to hypertension, platelet activation, increased cell proliferation, endothelial dysfunction, atherosclerosis, angina pectoris, heart failure, myocardial infarction, thromboses, stroke and sexual dysfunction.

Owing to the expected high efficiency and low level of side effects, a possible NO-independent treatment for such disorders by targeting the influence of the cGMP signal pathway in organisms is a promising approach.

Hitherto, for the therapeutic stimulation of the soluble guanylate cyclase, use has exclusively been made of compounds such as organic nitrates whose effect is based on NO. The latter is formed by bioconversion and activates soluble guanylate cyclase by attack at the central iron atom of haem. In addition to the side effects, the development of tolerance is one of the crucial disadvantages of this mode of treatment.

In recent years, some substances have been described which stimulate soluble guanylate cyclase directly, i.e. without prior release of NO, such as, for example, 3-(5′-hydroxymethyl-2′-furyl)-1-benzylindazole [YC-1; Wu et al., Blood 84 (1994), 4226; Mülsch et al., Brit. J. Pharmacol. 120 (1997), 681], fatty acids [Goldberg et al., J. Biol. Chem. 252 (1977), 1279], diphenyliodonium hexafluorophosphate [Pettibone et al., Eur. J. Pharmacol. 116 (1985), 307], isoliquiritigenin [Yu et al., Brit. J. Pharmacol. 114 (1995), 1587] and various substituted pyrazole derivatives (WO 98/16223).

Various imidazo[1,2-a]pyridine derivatives which can be used for treating disorders are described, inter alia, in EP 0 266 890-A1, WO 89/03833-A1, JP 01258674-A [cf. Chem. Abstr. 112:178986], WO 96/34866-A1, EP 1 277 754-A1, WO 2001/096335, WO 2006/015737-A1, WO 2006/135667, WO 2008/008539-A2, WO 2008/082490-A2, WO 2008/134553-A1, WO 2010/030538-A2, WO 2011/113606-A1 and WO 2012/165399-A1.

It was an object of the present invention to provide novel substances which act as stimulators of soluble guanylate cyclase and are suitable as such for the treatment and/or prophylaxis of diseases.

The present invention provides compounds of the general formula (I)

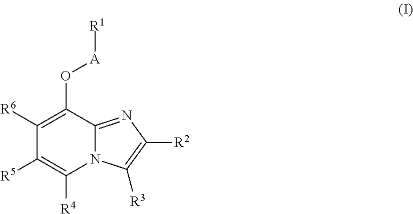

in which
A represents CH₂, CD₂ or CH(CH₃),
R¹ represents (C₃-C₇)-cycloalkyl, phenyl or pyridyl,
   where (C₃-C₇)-cycloalkyl may be substituted by 1 to 4 substituents independently of one another selected from the group consisting of fluorine, trifluoromethyl and (C₁-C₄)-alkyl,
   where phenyl is substituted by 1 to 4 substituents independently of one another selected from the group consisting of halogen, cyano, monofluoromethyl, difluoromethyl, trifluoromethyl, (C₁-C₄)-alkyl, (C₁-C₄)-alkoxy and difluoromethoxy
   and
   where pyridyl is substituted by 1 or 2 substituents independently of one another selected from the group consisting of halogen, cyano and (C₁-C₄)-alkyl,
R² represents (C₁-C₄)-alkyl, cyclopropyl, cyclobutyl, monofluoromethyl, difluoromethyl or trifluoromethyl,
R³ represents a group of the formula

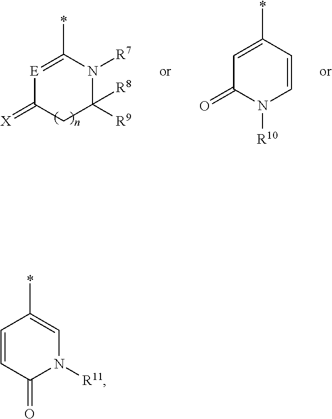

where
* represents the point of attachment to the imidazo[1,2-a]pyridine ring,
E represents carbon or nitrogen,
n represents 0, 1 or 2,
X represents oxygen or nitrogen,
   in which nitrogen may be substituted by hydrogen or hydroxy,
R⁷ represents hydrogen or (C₁-C₆)-alkyl,
   in which (C₁-C₆)-alkyl may be substituted by amino or hydroxy,
   and
   in which (C₁-C₆)-alkyl may be substituted up to five times by fluorine,
R⁸ represents hydrogen or (C₁-C₄)-alkyl,
   in which (C₁-C₄)-alkyl may be substituted up to five times by fluorine,
R⁹ represents hydrogen or (C₁-C₄)-alkyl,
   in which (C₁-C₄)-alkyl may be substituted up to five times by fluorine,
or
R⁸ and R⁹ together with the carbon atom to which they are bonded form a 3- to 7-membered carbocycle or a 4- to 7-membered heterocycle,
   in which the 3- to 7-membered carbocycle and the 4- to 7-membered heterocycle may in turn be substituted by 1 or 2 substituents independently of one another selected from the group consisting of fluorine and (C₁-C₄)-alkyl,
R¹⁰ represents hydrogen or (C₁-C₈)-alkyl,
   in which (C₁-C₈)-alkyl may be substituted by amino or hydroxy,
   and
   in which (C₁-C₈)-alkyl may be substituted up to five times by fluorine,
R¹¹ represents hydrogen or (C₁-C₈)-alkyl,
   in which (C₁-C₈)-alkyl may be substituted by amino or hydroxy,
   and
   in which (C₁-C₈)-alkyl may be substituted up to five times by fluorine,
or
represents 5- to 10-membered heteroaryl,
where 5- to 10-membered heteroaryl is substituted by (C₁-C₈)-alkoxy,
   in which (C₁-C₈)-alkoxy is substituted by amino,
   and
   in which (C₁-C₈)-alkoxy may be substituted up to five times by fluorine,
and
where 5- to 10-membered heteroaryl may be substituted by 1 or 2 substituents independently of one another selected from the group consisting of halogen, cyano, trifluoromethyl, difluoromethyl and (C₁-C₆)-alkyl,
R⁴ represents hydrogen,
R⁵ represents hydrogen, halogen, cyano, (C₁-C₄)-alkyl, (C₂-C₄)-alkynyl, (C₁-C₄)-alkoxy, (C₃-C₅)-cycloalkyl, difluoromethoxy, difluoromethyl, trifluoromethyl, 4- to 7-membered heterocyclyl or 5- or 6-membered heteroaryl,
R⁶ represents hydrogen or halogen, and the N-oxides, salts, solvates, salts of the N-oxides and solvates of the N-oxides and salts thereof.

Compounds of the invention are the compounds of the formula (I) and the salts, solvates and solvates of the salts thereof, the compounds that are encompassed by formula (I) and are of the formulae mentioned below and the salts, solvates and solvates of the salts thereof and the compounds that are encompassed by formula (I) and are mentioned below as working examples and the salts, solvates and solvates of the salts thereof if the compounds that are encompassed by formula (I) and are mentioned below are not already salts, solvates and solvates of the salts.

Preferred salts in the context of the present invention are physiologically acceptable salts of the compounds of the invention. Also encompassed are salts which are not themselves suitable for pharmaceutical applications but can be used, for example, for isolation or purification of the compounds according to the invention.

Physiologically acceptable salts of the compounds of the invention include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, formic acid, acetic acid, trifluoroacetic acid, propionic acid, lactic acid, tartaric acid, malic acid, citric acid, fumaric acid, maleic acid and benzoic acid.

Physiologically acceptable salts of the compounds of the invention also include salts of conventional bases, by way of example and with preference alkali metal salts (e.g. sodium and potassium salts), alkaline earth metal salts (e.g. calcium and magnesium salts) and ammonium salts derived from ammonia or organic amines having 1 to 16 carbon atoms, by way of example and with preference ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

Solvates in the context of the invention are described as those forms of the compounds of the invention which form a complex in the solid or liquid state by coordination with solvent molecules. Hydrates are a specific form of the solvates in which the coordination is with water. Solvates preferred in the context of the present invention are hydrates.

The compounds according to the invention may, depending on their structure, exist in different stereoisomeric forms, i.e. in the form of configurational isomers or else, if appropriate, as conformational isomers (enantiomers and/or diastereomers, including those in the case of atropisomers). The present invention therefore encompasses the enantiomers and diastereomers, and the respective mixtures thereof. The stereoisomerically homogeneous constituents can be isolated from such mixtures of enantiomers and/or diastereomers in a known manner; chromatographic processes are preferably used for this purpose, especially HPLC chromatography on an achiral or chiral phase.

If the compounds according to the invention can occur in tautomeric forms, the present invention encompasses all the tautomeric forms.

The present invention also encompasses all suitable isotopic variants of the compounds according to the invention. An isotopic variant of a compound according to the invention is understood here to mean a compound in which at least one atom within the compound according to the invention has been exchanged for another atom of the same atomic number, but with a different atomic mass from the atomic mass which usually or predominantly occurs in nature. Examples of isotopes which can be incorporated into a compound according to the invention are those of hydrogen, carbon, nitrogen, oxygen, phosphorus, sulphur, fluorine, chlorine, bromine and iodine, such as ²H (deuterium), ³H (tritium), ¹³C, ¹⁴C, ¹⁵N, ¹⁷O, ¹⁸O, ³²F, ³³F, ³³S, ³⁴S, ³⁵S, ³⁶S, ¹⁸F, ³⁶Cl, ⁸²Br, ¹²³I, ¹²⁴I, ¹²⁹I and ¹³¹I. Particular isotopic variants of a compound according to the invention, especially those in which one or more radioactive isotopes have been incorporated, may be beneficial, for example, for the examination of the mechanism of action or of the active compound distribution in the body; due to comparatively easy preparability and detectability, especially compounds labelled with ³H or ¹⁴C isotopes are suitable for this purpose. In addition, the incorporation of isotopes, for example of deuterium, may lead to particular therapeutic benefits as a consequence of greater metabolic stability of the compound, for example an extension of the half-life in the body or a reduction in the active dose required; such modifications of the compounds according to the invention may therefore in some cases also constitute a preferred embodiment of the present invention. Isotopic variants of the compounds of the invention can be prepared by the processes known to those skilled in the art, for example by the methods described further down and the procedures described in the working examples, by using corresponding isotopic modifications of the respective reagents and/or starting materials.

The present invention additionally also encompasses prodrugs of the compounds according to the invention. The term “prodrugs” in this context refers to compounds which may themselves be biologically active or inactive but are reacted (for example metabolically or hydrolytically) to give compounds according to the invention during their residence time in the body.

In the context of the present invention, unless specified otherwise, the substituents are defined as follows:

Alkyl in the context of the invention is a straight-chain or branched alkyl radical having the particular number of carbon atoms specified. The following may be mentioned by way of example and by way of preference: methyl, ethyl, n-propyl, isopropyl, n-butyl, isobutyl, 1-methylpropyl, tert-butyl, n-pentyl, isopentyl, 1-ethylpropyl, 1-methylbutyl, 2-methylbutyl, 3-methylbutyl, n-hexyl, 1-methylpentyl, 2-methylpentyl, 3-methylpentyl, 4-methylpentyl, 3,3-dimethylbutyl, 1-ethylbutyl, 2-ethylbutyl.

Cycloalkyl or carbocycle in the context of the invention represents a monocyclic saturated alkyl radical having the particular number of ring carbon atoms specified. The following may be mentioned by way of example and by way of preference: cyclopropyl, cyclobutyl, cyclopentyl, cyclohexyl and cycloheptyl.

Alkoxy in the context of the invention is a straight-chain or branched alkoxy radical having 1 to 4 carbon atoms. The following may be mentioned by way of example and by way of preference: methoxy, ethoxy, n-propoxy, isopropoxy, 1-methylpropoxy, n-butoxy, isobutoxy and tert-butoxy.

Alkoxycarbonyl in the context of the invention is a straight-chain or branched alkoxy radical having 1 to 4 carbon atoms and a carbonyl group attached to the oxygen atom. The following may be mentioned by way of example and by way of preference: methoxycarbonyl, ethoxycarbonyl, n-propoxycarbonyl, isopropoxycarbonyl and tert-butoxycarbonyl.

Alkylsulphonyl in the context of the invention is a straight-chain or branched alkyl radical which has 1 to 4 carbon atoms and is bonded via a sulphonyl group. The following may be mentioned by way of example and by way of preference: methylsulphonyl, ethylsulphonyl, n-propylsulphonyl, isopropylsulphonyl, n-butylsulphonyl and tert-butylsulphonyl.

A 4- to 7-membered heterocycle or 4- to 7-membered heterocyclyl in the context of the invention is a monocyclic saturated heterocycle which has a total of 4 to 7 ring atoms, contains one or two ring heteroatoms from the group consisting of N, O, S, SO and SO₂ and is joined via a ring carbon atom or optionally a ring nitrogen atom. The following may be mentioned by way of example: azetidinyl, oxetanyl, pyrrolidinyl, pyrazolidinyl, tetrahydrofuranyl, thiolanyl, piperidinyl, piperazinyl, tetrahydropyranyl, tetrahydrothiopyranyl, morpholinyl, thiomorpholinyl, hexahydroazepinyl and hexahydro-1,4-diazepinyl. Preference is given to azetidinyl, oxetanyl, pyrrolidinyl, tetrahydrofuranyl, piperidinyl, piperazinyl, tetrahydropyranyl and morpholinyl.

Heteroaryl in the context of the invention represents a monocyclic aromatic heterocycle (heteroaromatic) which has a total of 5 to 10 ring atoms, contains up to three identical or different ring heteroatoms from the group of N, O and/or S and is attached via a ring carbon atom or optionally via a ring nitrogen atom. The following may be mentioned by way of example and by way of preference: furyl, pyrrolyl, thienyl, 1H-pyrazol-4-yl, 1H-pyrazol-5-yl, imidazolyl, 1,3-thiazol-5-yl, 1,3-thiazol-2-yl, 1,3-oxazol-5-yl, 1,3-oxazol-2-yl, isoxazolyl, isothiazolyl, triazolyl, 1,3,4-oxadiazol-2-yl, 1,2,4-oxadiazol-3-yl, 1,2,4-oxadiazol-5-yl, 1,3,4-thiadiazol-2-yl, 1,2,4-thiadiazol-3-yl, 1,2,4-thiadiazol-5-yl, pyridyl, pyrimidinyl, pyridazinyl, pyrazinyl and triazinyl.

Halogen in the context of the invention includes fluorine, chlorine, bromine and iodine. Preference is given to chlorine or fluorine.

In the formula of the group that R³ or R¹ may represent, the end point of the line marked by the symbol *, # or ## does not represent a carbon atom or a CH₂ group but is part of the bond to the respectively marked atom to which R³ or R¹ is attached.

When radicals in the compounds according to the invention are substituted, the radicals may be mono- or polysubstituted, unless specified otherwise. In the context of the present invention, all radicals which occur more than once are defined independently of one another. Substitution by one, two or three identical or different substituents is preferred.

In the context of the present invention, the term “treatment” or “treating” includes inhibition, retardation, checking, alleviating, attenuating, restricting, reducing, suppressing, repelling or healing of a disease, a condition, a disorder, an injury or a health problem, or the development, the course or the progression of such states and/or the symptoms of such states. The term “therapy” is understood here to be synonymous with the term “treatment”.

The terms “prevention”, “prophylaxis” and “preclusion” are used synonymously in the context of the present invention and refer to the avoidance or reduction of the risk of contracting, experiencing, suffering from or having a disease, a condition, a disorder, an injury or a health problem, or a development or advancement of such states and/or the symptoms of such states.

The treatment or prevention of a disease, a condition, a disorder, an injury or a health problem may be partial or complete.

In the context of the present invention, preference is given to compounds of the formula (I) in which
A represents CH₂ or CD₂,
R¹ represents cyclohexyl, phenyl or pyridyl,
   where phenyl is substituted by 1 to 4 substituents independently of one another selected from the group consisting of fluorine, bromine, chlorine, cyano and methyl,
   and
   where pyridyl is substituted by 1 or 2 substituents independently of one another selected from the group consisting of fluorine, cyano and methyl,
R² represents (C₁-C₄)-alkyl, cyclopropyl or trifluoromethyl,
R³ represents a group of the formula

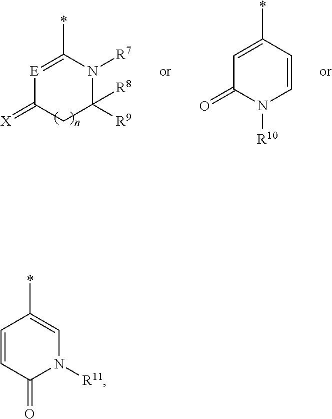

where
* represents the point of attachment to the imidazo[1,2-a]pyridine ring,
E represents carbon or nitrogen,
n represents 0 or 1,
X represents oxygen or nitrogen,
   in which nitrogen may be substituted by hydrogen or hydroxy,
R⁷ represents hydrogen or (C₁-C₆)-alkyl,
   in which (C₁-C₆)-alkyl may be substituted by amino or hydroxy,
   and
   in which (C₁-C₆)-alkyl may be substituted up to five times by fluorine,
R⁸ represents hydrogen or (C₁-C₄)-alkyl,
   in which (C₁-C₄)-alkyl may be substituted up to five times by fluorine,
R⁹ represents hydrogen or (C₁-C₄)-alkyl,
   in which (C₁-C₄)-alkyl may be substituted up to five times by fluorine,
or
R⁸ and R⁹ together with the carbon atom to which they are bonded form a 3- to 7-membered carbocycle,
   in which the 3- to 7-membered carbocycle may be substituted by 1 or 2 substituents independently of one another selected from the group consisting of fluorine and methyl,
R¹⁰ represents hydrogen or (C₁-C₈)-alkyl,
   in which (C₁-C₈)-alkyl may be substituted by amino or hydroxy,
   and
   in which (C₁-C₈)-alkyl may be substituted up to five times by fluorine,
R¹¹ represents hydrogen or (C₁-C₈)-alkyl,
   in which (C₁-C₈)-alkyl may be substituted by amino or hydroxy,
   and
   in which (C₁-C₈)-alkyl may be substituted up to five times by fluorine,
or
represent a group of the formula

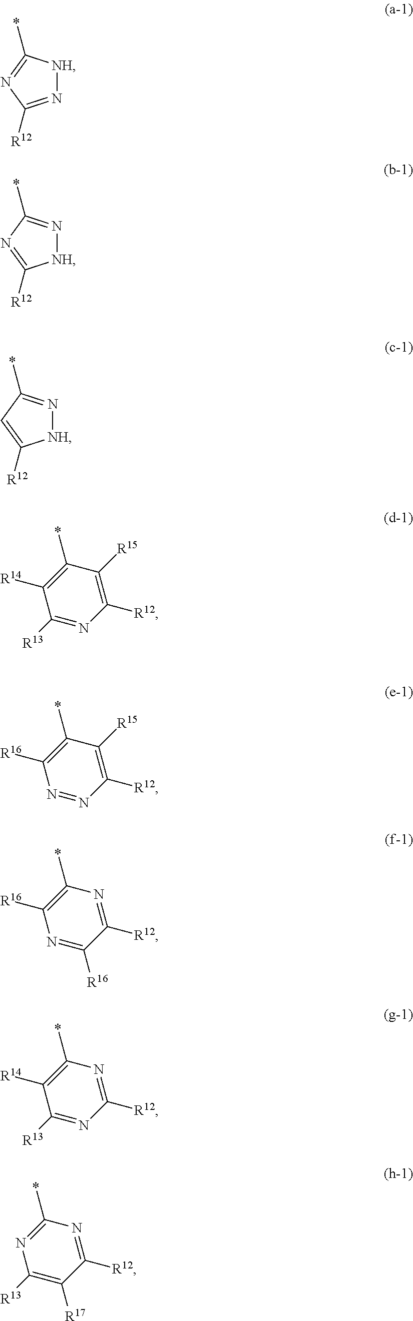

where
R¹² represents (C₁-C₈)-alkoxy,
   in which (C₁-C₈)-alkoxy is substituted by amino,
   and
   in which (C₁-C₈)-alkoxy may be substituted up to five times by fluorine,
and
R¹³ represents hydrogen, cyano, trifluoromethyl, difluoromethyl or methyl,
R¹⁴ represents hydrogen, fluorine, chlorine, cyano, trifluoromethyl, difluoromethyl or methyl,
R¹⁵ represents hydrogen, fluorine, chlorine, cyano, trifluoromethyl, difluoromethyl or methyl,
R¹⁶ represents hydrogen, cyano, trifluoromethyl, difluoromethyl or methyl,
R¹⁷ represents hydrogen, fluorine, chlorine, cyano, trifluoromethyl, difluoromethyl or methyl,
R⁴ represents hydrogen,
R⁵ represents hydrogen, chlorine, cyano, methyl, methoxy or cyclopropyl,
R⁶ represents hydrogen or fluorine,
   and the N-oxides, salts, solvates, salts of the N-oxides and solvates of the N-oxides and salts thereof.

In the context of the present invention, preference is given to compounds of the formula (I) in which
A represents CH₂,
R¹ represents a phenyl group of the formula

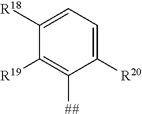

where
## represents the point of attachment to A,
and
R¹⁸, R¹⁹ and R²⁰ independently of one another represent hydrogen or fluorine, with the proviso that at least two of the radicals R¹⁸, R¹⁹, R²⁰ are different from hydrogen,
R² represents methyl,
R³ represents a group of the formula

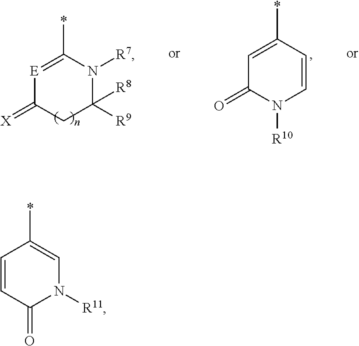

where
* represents the point of attachment to the imidazo[1,2-a]pyridine ring,
E represents carbon or nitrogen,
n represents 1,
X represents oxygen or nitrogen,
   in which nitrogen may be substituted by hydrogen or hydroxy,
R⁷ represents hydrogen,
R⁸ represents hydrogen, methyl or ethyl,
   in which methyl may be substituted up to three times by fluorine,
   and
   in which ethyl may be substituted up to five times by fluorine,
R⁹ represents hydrogen, methyl or ethyl,
   in which methyl may be substituted up to three times by fluorine,
   and
   in which ethyl may be substituted up to five times by fluorine,
or
R⁸ and R⁹ together with the carbon atom to which they are bonded form a 3- to 6-membered carbocycle,
R¹⁰ represents hydrogen or (C₁-C₈)-alkyl,
   in which (C₁-C₈)-alkyl is substituted by amino,
   and
   in which (C₁-C₈)-alkyl may be substituted up to five times by fluorine,
R¹¹ represents hydrogen or (C₁-C₈)-alkyl,
   in which (C₁-C₈)-alkyl is substituted by amino,
   and
   in which (C₁-C₈)-alkyl may be substituted up to five times by fluorine,
or
represent a group of the formula

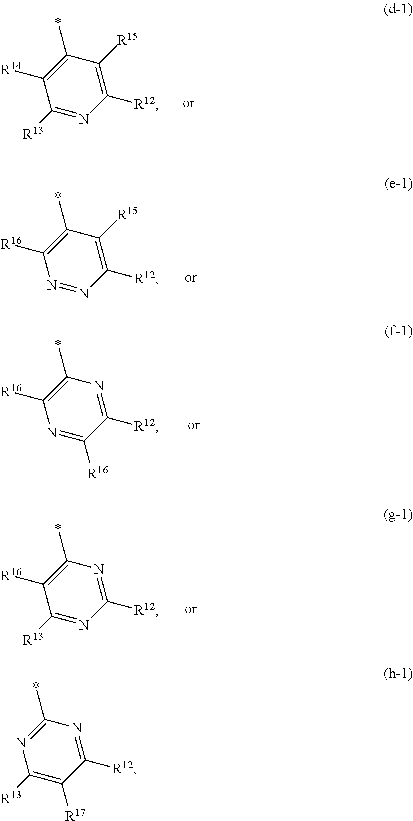

where
R¹² represents (C₁-C₈)-alkoxy,
   in which (C₁-C₈)-alkoxy is substituted by amino,
   and
   in which (C₁-C₈)-alkoxy may be substituted up to five times by fluorine,
R¹³ represents hydrogen or methyl,
R¹⁴ represents hydrogen, fluorine, chlorine or methyl,
R¹⁵ represents hydrogen, fluorine, chlorine or methyl,
R¹⁶ represents hydrogen or methyl,
and
R¹⁷ represents hydrogen, fluorine, chlorine or methyl,
R⁴ represents hydrogen,
R⁵ represents hydrogen, chlorine or methyl,
R⁶ represents hydrogen,
   and the N-oxides, salts, solvates, salts of the N-oxides and solvates of the N-oxides and salts thereof.

In the context of the present invention, preference is given to compounds of the formula (I) in which
A represents CH₂,
R¹ represents a phenyl group of the formula

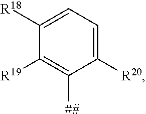

where
## represents the point of attachment to A,
and
R¹⁸, R¹⁹ and R²⁰ independently of one another represent hydrogen or fluorine, with the proviso that at least two of the radicals R¹⁸, R¹⁹, R²⁰ are different from hydrogen,
R² represents methyl,
R³ represents a group of the formula

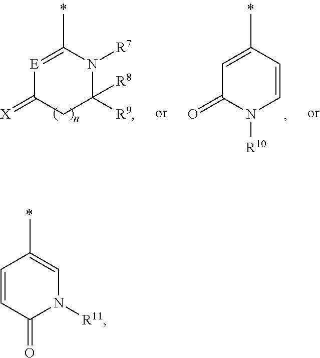

where
* represents the point of attachment to the imidazo[1,2-a]pyridine ring,
E represents carbon or nitrogen,
n represents 1,
X represents oxygen or nitrogen,
   in which nitrogen may be substituted by hydrogen or hydroxy,
R⁷ represents hydrogen,
R⁸ represents hydrogen, methyl or ethyl,
   in which methyl may be substituted up to three times by fluorine,
   and
   in which ethyl may be substituted up to five times by fluorine,
R⁹ represents hydrogen, methyl or ethyl,
   in which methyl may be substituted up to three times by fluorine,
   and
   in which ethyl may be substituted up to five times by fluorine
or
R⁸ and R⁹ together with the carbon atom to which they are attached form a cyclopropyl ring,
R¹⁰ represents hydrogen or (C₁-C₆)-alkyl,
   in which (C₁-C₆)-alkyl is substituted by amino,
   and
   in which (C₁-C₆)-alkyl may be substituted up to five times by fluorine,
R¹¹ represents hydrogen or (C₁-C₆)-alkyl,
   in which (C₁-C₆)-alkyl is substituted by amino,
   and
   in which (C₁-C₆)-alkyl may be substituted up to five times by fluorine,
or
represent a group of the formula

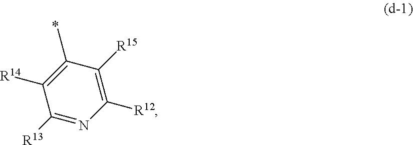

where
R¹² represents (C₁-C₆)-alkoxy,
   in which (C₁-C₆)-alkoxy is substituted by amino,
   and
   in which (C₁-C₆)-alkoxy may be substituted up to five times by fluorine,
R¹³ represents hydrogen,
R¹⁴ represents hydrogen or fluorine,
R¹⁵ represents hydrogen or fluorine,
R⁴ represents hydrogen,
R⁵ represents hydrogen, chlorine or methyl,
R⁶ represents hydrogen,
   and the N-oxides, salts, solvates, salts of the N-oxides and solvates of the N-oxides and salts thereof.

In the context of the present invention, preference is also given to compounds of the formula (I) in which
A represents CH₂,
   and the N-oxides, salts, solvates, salts of the N-oxides and solvates of the N-oxides and salts thereof.

In the context of the present invention, preference is also given to compounds of the formula (I) in which
R¹ represents a phenyl group of the formula

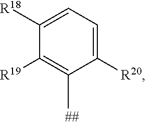

where
## represents the point of attachment to A,
and
R¹⁸, R¹⁹ and R²⁰ independently of one another represent hydrogen or fluorine, with the proviso that at least two of the radicals R¹⁸, R¹⁹, R²⁰ are different from hydrogen,
   and the N-oxides, salts, solvates, salts of the N-oxides and solvates of the N-oxides and salts thereof.

In the context of the present invention, preference is also given to compounds of the formula (I) in which
R¹ represents a phenyl group of the formula

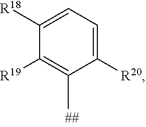

where
## represents the point of attachment to A,
and
R¹⁸ represents hydrogen,
and
R¹⁹ and R²⁰ represent fluorine,
   and the N-oxides, salts, solvates, salts of the N-oxides and solvates of the N-oxides and salts thereof.

In the context of the present invention, preference is also given to compounds of the formula (I) in which
R² represents methyl,
   and the N-oxides, salts, solvates, salts of the N-oxides and solvates of the N-oxides and salts thereof.

In the context of the present invention, preference is also given to compounds of the formula (I) in which
R³ represents a group of the formula

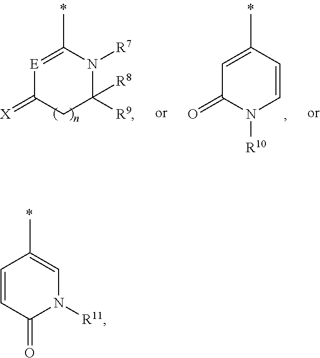

where
* represents the point of attachment to the imidazo[1,2-a]pyridine ring,
E represents carbon or nitrogen,
n represents 1,
X represents oxygen or nitrogen,
   in which nitrogen may be substituted by hydrogen or hydroxy,
R⁷ represents hydrogen,
R⁸ represents hydrogen, methyl or ethyl,
   in which methyl may be substituted up to three times by fluorine,
   and
   in which ethyl may be substituted up to five times by fluorine,
R⁹ represents hydrogen, methyl or ethyl,
   in which methyl may be substituted up to three times by fluorine,
   and
   in which ethyl may be substituted up to five times by fluorine
or
R⁸ and R⁹ together with the carbon atom to which they are attached form a cyclopropyl ring,
R¹⁰ represents hydrogen or (C₁-C₆)-alkyl,
   in which (C₁-C₆)-alkyl is substituted by amino,
   and
   in which (C₁-C₆)-alkyl may be substituted up to five times by fluorine,
R¹¹ represents hydrogen or (C₁-C₆)-alkyl,
   in which (C₁-C₆)-alkyl is substituted by amino,
   and
   in which (C₁-C₆)-alkyl may be substituted up to five times by fluorine,
or
represent a group of the formula

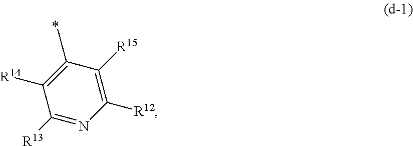

where
R¹² represents (C₁-C₆)-alkoxy,
   in which (C₁-C₆)-alkoxy is substituted by amino,
   and
   in which (C₁-C₆)-alkoxy may be substituted up to five times by fluorine,
R¹³ represents hydrogen,
R¹⁴ represents hydrogen or fluorine,
R¹⁵ represents hydrogen or fluorine,
and the N-oxides, salts, solvates, salts of the N-oxides and solvates of the N-oxides and salts thereof.

In the context of the present invention, preference is also given to compounds of the formula (I) in which
R³ represents a group of the formula

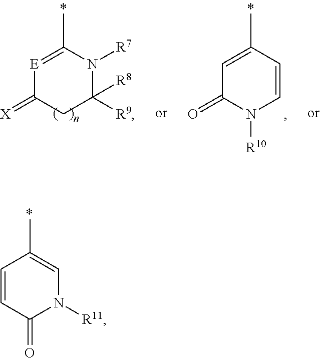

where
* represents the point of attachment to the imidazo[1,2-a]pyridine ring,
E represents carbon or nitrogen,
n represents 1,
X represents oxygen or nitrogen,
   in which nitrogen may be substituted by hydrogen or hydroxy,
R⁷ represents hydrogen,
R⁸ represents hydrogen, methyl or ethyl,
   in which methyl may be substituted up to three times by fluorine,
   and
   in which ethyl may be substituted up to five times by fluorine,
R⁹ represents hydrogen, methyl or ethyl,
   in which methyl may be substituted up to three times by fluorine,
   and
   in which ethyl may be substituted up to five times by fluorine
or
R⁸ and R⁹ together with the carbon atom to which they are attached form a cyclopropyl ring,
R¹⁰ represents hydrogen or (C₁-C₆)-alkyl,
   in which (C₁-C₆)-alkyl is substituted by amino,
   and
   in which (C₁-C₆)-alkyl may be substituted up to five times by fluorine,
R¹¹ represents hydrogen or (C₁-C₆)-alkyl,
   in which (C₁-C₆)-alkyl is substituted by amino,
   and
   in which (C₁-C₆)-alkyl may be substituted up to five times by fluorine,
      and the N-oxides, salts, solvates, salts of the N-oxides and solvates of the N-oxides and salts thereof.

In the context of the present invention, preference is also given to compounds of the formula (I) in which
R³ represents a group of the formula

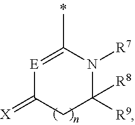

where
* represents the point of attachment to the imidazo[1,2-a]pyridine ring,
E represents carbon or nitrogen,
n represents 1,
X represents oxygen or nitrogen,
   in which nitrogen may be substituted by hydrogen or hydroxy,
R⁷ represents hydrogen,
R⁸ represents hydrogen, methyl or ethyl,
   in which methyl may be substituted up to three times by fluorine,
   and
   in which ethyl may be substituted up to five times by fluorine,
R⁹ represents hydrogen, methyl or ethyl,
   in which methyl may be substituted up to three times by fluorine,
   and
   in which ethyl may be substituted up to five times by fluorine
or
R⁸ and R⁹ together with the carbon atom to which they are attached form a cyclopropyl ring,
   and the N-oxides, salts, solvates, salts of the N-oxides and solvates of the N-oxides and salts thereof.

In the context of the present invention, preference is also given to compounds of the formula (I) in which
R³ represents a group of the formula

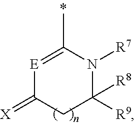

where
* represents the point of attachment to the imidazo[1,2-a]pyridine ring,
E represents carbon or nitrogen,
n represents 1,
X represents nitrogen,
   in which nitrogen is substituted by hydroxy,
R⁷ represents hydrogen,
R⁸ represents methyl or ethyl,
   in which methyl may be substituted up to three times by fluorine,
   and
   in which ethyl may be substituted up to five times by fluorine,
R⁹ represents hydrogen, methyl or ethyl,
   in which methyl may be substituted up to three times by fluorine,
   and
   in which ethyl may be substituted up to five times by fluorine
      and the N-oxides, salts, solvates, salts of the N-oxides and solvates of the N-oxides and salts thereof.

In the context of the present invention, preference is also given to compounds of the formula (I) in which
R³ represents a group of the formula

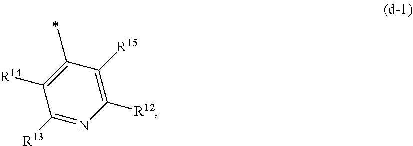

where
R¹² represents (C₁-C₆)-alkoxy,
   in which (C₁-C₆)-alkoxy is substituted by amino,
   and
   in which (C₁-C₆)-alkoxy may be substituted up to five times by fluorine,
R¹³ represents hydrogen,
R¹⁴ represents hydrogen or fluorine,
R¹⁵ represents hydrogen or fluorine,
and the N-oxides, salts, solvates, salts of the N-oxides and solvates of the N-oxides and salts thereof.

In the context of the present invention, preference is also given to compounds of the formula (I) in which
R³ represents a group of the formula

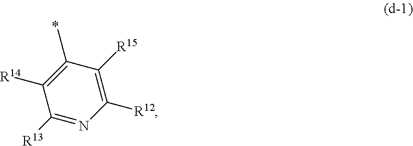

where
R¹² represents (C₁-C₆)-alkoxy,
   in which (C₁-C₆)-alkoxy is substituted by amino,
   and
   in which (C₁-C₆)-alkoxy may be substituted up to five times by fluorine,
R¹³ represents hydrogen,
R¹⁴ represents hydrogen,
R¹⁵ represents hydrogen,
and the N-oxides, salts, solvates, salts of the N-oxides and solvates of the N-oxides and salts thereof.

In the context of the present invention, preference is also given to compounds of the formula (I) in which
R⁵ represents hydrogen, chlorine or methyl,
and the N-oxides, salts, solvates, salts of the N-oxides and solvates of the N-oxides and salts thereof.

In the context of the present invention, preference is also given to compounds of the formula (I) in which
R⁵ represents hydrogen,
and the N-oxides, salts, solvates, salts of the N-oxides and solvates of the N-oxides and salts thereof.

In the context of the present invention, preference is also given to compounds of the formula (I) in which
R⁵ represents chlorine,
and the N-oxides, salts, solvates, salts of the N-oxides and solvates of the N-oxides and salts thereof.

In the context of the present invention, preference is also given to compounds of the formula (I) in which
R⁵ represents methyl,
and the N-oxides, salts, solvates, salts of the N-oxides and solvates of the N-oxides and salts thereof.

The individual radical definitions specified in the respective combinations or preferred combinations of radicals are, independently of the respective combinations of the radicals specified, also replaced as desired by radical definitions of other combinations.

Particular preference is given to combinations of two or more of the preferred ranges mentioned above.

The invention further provides a process for preparing the compounds of the formula (I) according to the invention, characterized in that

a compound of the formula (II)

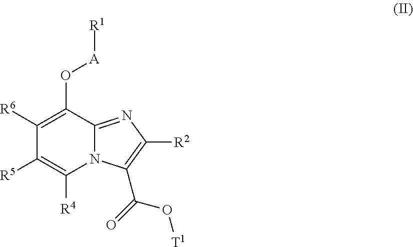

in which A, R¹, R², R⁴, R⁵ and R⁶ are each as defined above and
T¹ represents (C₁-C₄)-alkyl or benzyl,
   is reacted in an inert solvent in the presence of a suitable base or acid to give a carboxylic acid of the formula (III)

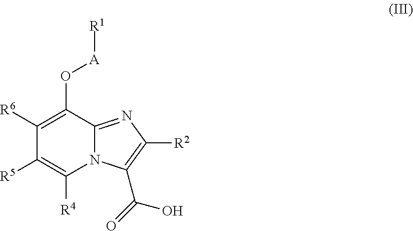

in which A, R¹, R², R⁴, R⁵ and R⁶ each have the meanings given above,
and these are subsequently reacted in the presence of a suitable acid to give an imidazo[1,2-a]-pyridine of the formula (IV)

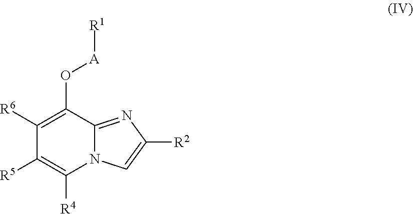

in which A, R¹, R², R⁴, R⁵ and R⁶ each have the meanings given above,
and this is then converted with a halogen equivalent into a compound of the formula (V)

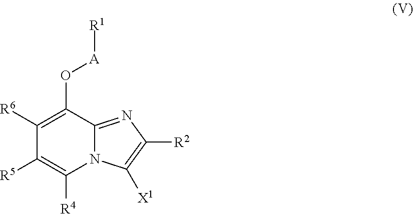

in which A, R¹, R², R⁴, R⁵ and R⁶ are each as defined above and
X¹ represents chlorine, bromine or iodine,
   and this is subsequently reacted in an inert solvent, in the presence of a suitable transition metal catalyst, with a compound of the formula (VI)

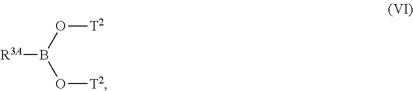

in which
R^{3A} has the meanings given above for R³
   and
T² represents hydrogen or (C₁-C₄)-alkyl, or the two T² radicals together form a —C(CH₃)₂—C(CH₃)₂— bridge,
   to give a compound of the formula (I-A)

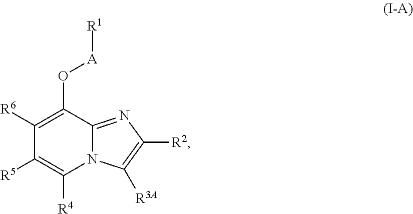

and these compounds are subsequently, if R^{3A} represents

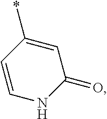 [SMILES: *c1cc[nH]c(=O)c1]

reacted in an inert solvent in the presence of a suitable base with a compound of the formula (VIII)

R^{10A}—X² (VIII)

in which
X² represents a suitable leaving group, in particular chlorine, bromine, iodine, mesylate, triflate or tosylate,
   and
R^{10A} represents (C₁-C₈)-alkyl,
   in which (C₁-C₈)-alkyl is substituted by nitro,
   and
   in which (C₁-C₈)-alkyl may be substituted up to five times by fluorine,
      to give compounds of the formula (VII-A) or (VII-B)

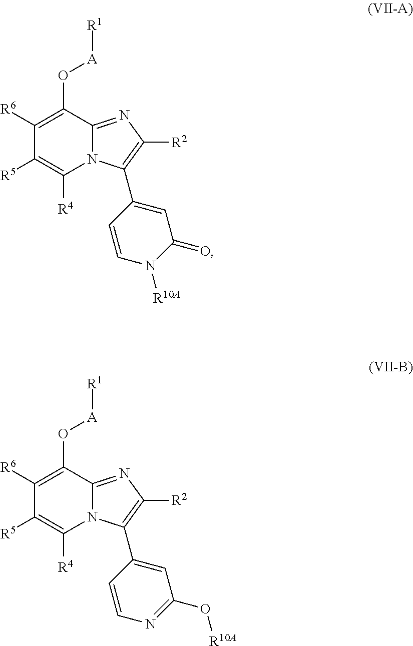

in which A, R¹, R², R⁴, R⁵ and R⁶ each have the meanings given above
and
R^{10A} represents (C₁-C₈)-alkyl,
   in which (C₁-C₈)-alkyl is substituted by nitro,
   and
   in which (C₁-C₈)-alkyl may be substituted up to five times by fluorine,
      and the nitro compounds are converted in an inert solvent in the presence of Raney nickel or palladium/carbon in a hydrogen atmosphere into compounds of the formula (I-B and I-C)

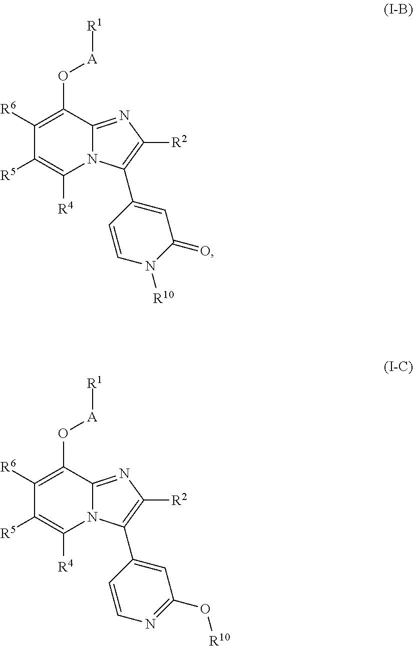

in which A, R¹, R², R⁴, R⁵, R⁶ and R¹⁰ each have the meanings given above,
any protective groups present are subsequently detached, and the resulting compounds of the formula (I) are optionally converted with the appropriate (i) solvents and/or (ii) acids or bases to the solvates, salts and/or solvates of the salts thereof.

The preparation processes described can be illustrated by way of example by the following synthesis schemes (Schemes 1 and 2):

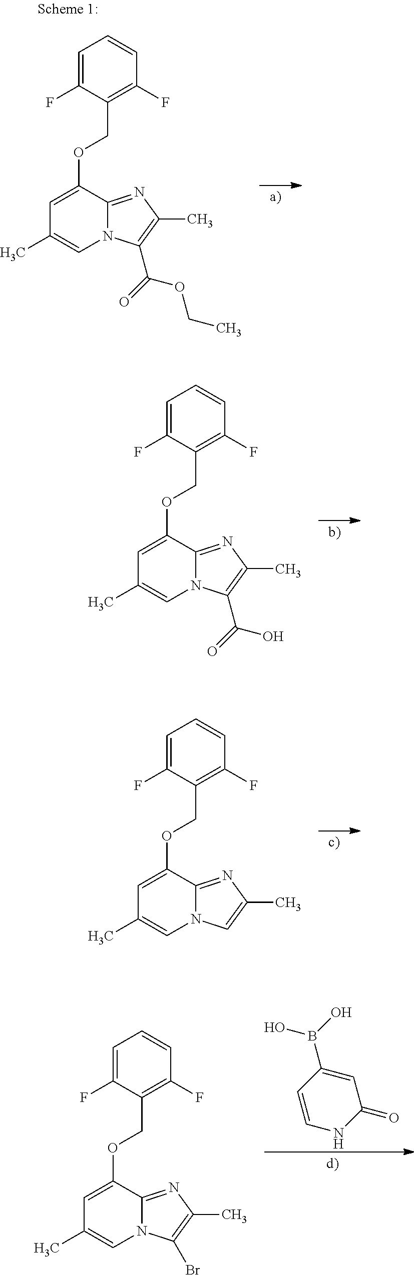

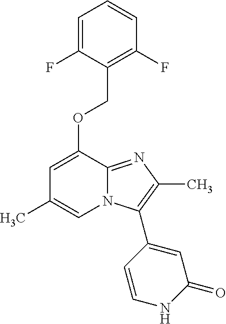 [SMILES: Cc1cc(OCc2c(F)cccc2F)c2nc(C)c(-c3cc[nH]c(=O)c3)n2c1]

[a): lithium hydroxide, THF/methanol/H₂O, RT; b): 6 N hydrochloric acid, 100° C.; c): N-bromosuccinimide, ethanol, RT; d): tetrakis(triphenylphosphine)palladium(0) (or [1,1′-bis(diphenylphosphino)ferrocene]dichloropalladium(II) complex), potassium phosphate (or sodium carbonate), ethanol/water/toluene, 90° C.].

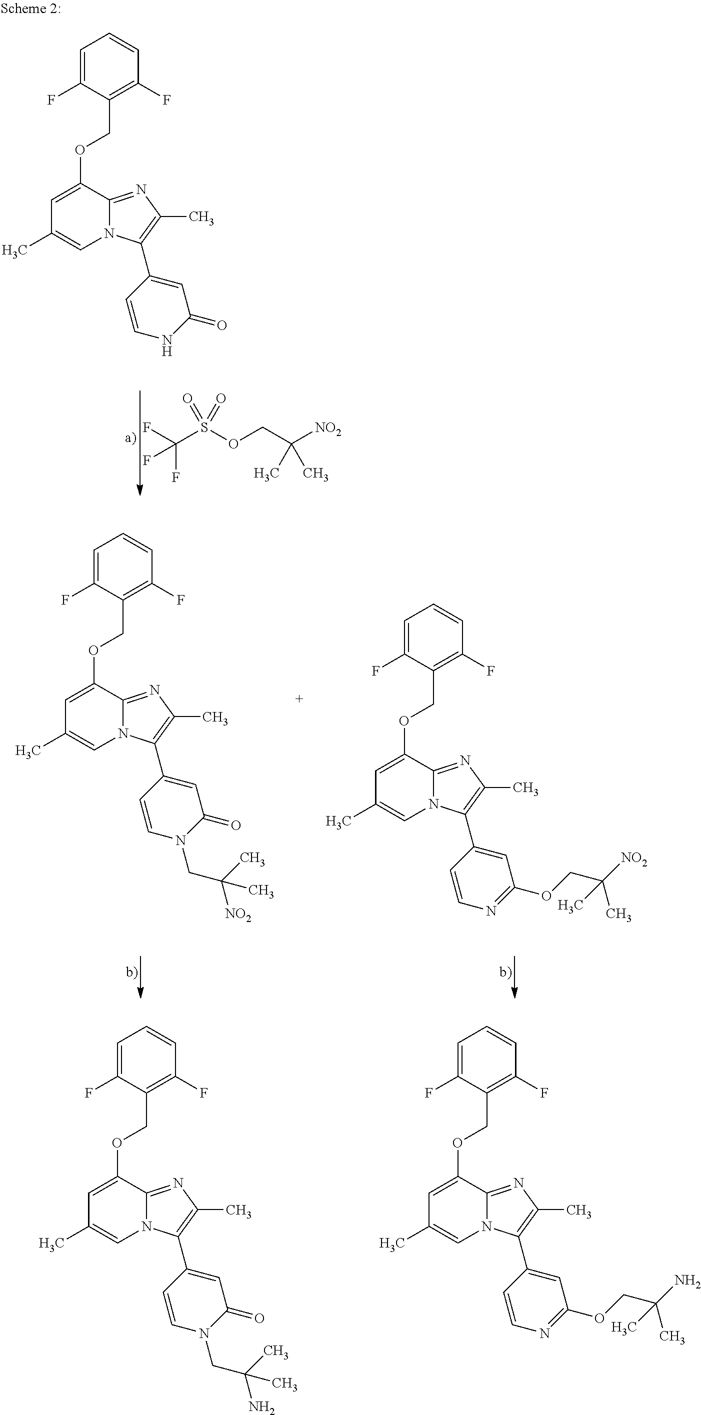 [SMILES: Cc1cc(OCc2c(F)cccc2F)c2nc(C)c(-c3cc[nH]c(=O)c3)n2c1 ; CC(C)(COS(=O)(=O)C(F)(F)F)[N+](=O)[O-] ; Cc1cc(OCc2c(F)cccc2F)c2nc(C)c(-c3ccn(CC(C)(C)[N+](=O)[O-])c(=O)c3)n2c1 ; Cc1cc(OCc2c(F)cccc2F)c2nc(C)c(-c3ccnc(OCC(C)(C)[N+](=O)[O-])c3)n2c1 ; Cc1cc(OCc2c(F)cccc2F)c2nc(C)c(-c3ccn(CC(C)(C)N)c(=O)c3)n2c1 ; Cc1cc(OCc2c(F)cccc2F)c2nc(C)c(-c3ccnc(OCC(C)(C)N)c3)n2c1]

[a): caesium carbonate, dioxane, RT; b): Raney nickel, EtOH, H₂, 1 bar, RT].

The compounds of the formulae (VI), (VIII), (IX) and (XI) are commercially available, known from the literature or can be prepared in analogy to literature processes.

The hydrolysis of the ester group T¹ in the compounds of the formula (II) is carried out by customary methods, by treating the esters in inert solvents with acids or bases, in which latter case the salts formed at first are converted to the free carboxylic acids by treating with acid. In the case of the tert-butyl esters, the ester hydrolysis is preferably carried out with acids. In the case of the benzyl esters, the ester hydrolysis is preferably carried out by hydrogenolysis with palladium on activated carbon or Raney nickel. Suitable inert solvents for this reaction are water or the organic solvents customary for ester hydrolysis. These preferably include alcohols such as methanol, ethanol, n-propanol, isopropanol, n-butanol or tert-butanol, or ethers such as diethyl ether, tetrahydrofuran, 2-methyltetrahydrofuran, dioxane or glycol dimethyl ether, or other solvents such as acetone, dichloromethane, dimethylformamide or dimethyl sulphoxide. It is also possible to use mixtures of the solvents mentioned. In the case of a basic ester hydrolysis, preference is given to using mixtures of water with dioxane, tetrahydrofuran, methanol and/or ethanol.

Suitable bases for the ester hydrolysis are the customary inorganic bases. These preferably include alkali metal or alkaline earth metal hydroxides, for example sodium hydroxide, lithium hydroxide, potassium hydroxide or barium hydroxide, or alkali metal or alkaline earth metal carbonates, such as sodium carbonate, potassium carbonate or calcium carbonate. Particular preference is given to sodium hydroxide or lithium hydroxide.

Suitable acids for the ester hydrolysis are generally sulphuric acid, hydrogen chloride/hydrochloric acid, hydrogen bromide/hydrobromic acid, phosphoric acid, acetic acid, trifluoroacetic acid, toluenesulphonic acid, methanesulphonic acid or trifluoromethanesulphonic acid, or mixtures thereof, optionally with addition of water. Preference is given to hydrogen chloride or trifluoroacetic acid in the case of the tert-butyl esters and to hydrochloric acid in the case of the methyl esters.

The ester hydrolysis is generally carried out within a temperature range from 0° C. to +100° C., preferably at +0° C. to +50° C.

These conversions can be performed at atmospheric, elevated or reduced pressure (for example from 0.5 to 5 bar). In general, the reaction is in each case carried out at atmospheric pressure.

Suitable solvents for the process step (III)→(IV) are water and dioxane. It is also possible to use mixtures of the solvents mentioned.

Suitable acids for the process step (III)→(IV) are hydrogen chloride/hydrochloric acid, hydrogen bromide/hydrobromic acid, sulphuric acid, acetic acid or mixtures thereof, optionally with addition of water. Preference is given to using hydrochloric acid.

The decarboxylation (III)→(IV) is generally carried out in a temperature range of from +20° C. to +100° C., preferably at from 75° C. to +100° C. The conversion can be carried out under atmospheric, elevated or reduced pressure (for example from 0.5 to 5 bar). In general, the reaction is carried out at atmospheric pressure.

Suitable solvents for process step (IV)→(V) include alcohols such as methanol, ethanol, n-propanol, isopropanol, n-butanol or tert-butanol, or ethers such as diethyl ether, tetrahydrofuran, 2-methyltetrahydrofuran, dioxane or glycol dimethyl ether, or other solvents such as acetone, dichloromethane, dimethylformamide or dimethyl sulphoxide. It is also possible to use mixtures of the solvents mentioned. Preference is given to using methanol and/or ethanol.

A suitable halogen source for the reaction (IV)→(V) is, for example, N-bromosuccinimide, N-chlorosuccinimide, N-iodosuccinimide, chlorine, bromine or iodine. Preference is given to using N-bromosuccinimide.

The reaction (IV)→(V) is generally carried out in a temperature range of from +20° C. to +100° C., preferably in the range from +20° C. to +80° C. The reaction can be performed at atmospheric, elevated or reduced pressure (for example in the range from 0.5 to 5 bar). In general, the reaction is carried out at atmospheric pressure.

Process step (V)+(VI)→(I-A) is carried out in a solvent which is inert under the reaction conditions. Suitable solvents are, for example, alcohols such as methanol, ethanol, n-propanol, isopropanol, n-butanol or tert-butanol, ethers such as diethyl ether, dioxane, tetrahydrofuran, glycol dimethyl ether or diethylene glycol dimethyl ether, or other solvents such as 1,2-dimethoxyethane (DME), dimethylformamide (DMF), dimethyl sulphoxide (DMSO), N,N′-dimethylpropyleneurea (DMPU), N-methylpyrrolidone (NMP), pyridine, acetonitrile, toluene or else water. It is also possible to use mixtures of the solvents mentioned. Preference is given to methanol, ethanol, toluene and water.

The conversion (V)+(VI)→(I-A) can optionally be carried out in the presence of a suitable palladium and/or copper catalyst. A suitable palladium catalyst is, for example, palladium(II) acetate, tetrakis(triphenylphosphine)palladium(0), bis(tri-tert-butylphosphine)palladium(0), bis(triphenylphosphine)palladium(II) chloride, bis(acetonitrile)palladium(II) chloride and [1,1′-bis(diphenylphosphino)ferrocene]dichloropalladium(II) and the corresponding dichloromethane complex, optionally in conjunction with additional phosphane ligands, for example (2-biphenyl)di-tert-butylphosphine, 2-dicyclohexylphosphino-2′,6′-dimethoxybiphenyl (SPHOS), dicyclohexyl[2′,4′,6′-tris(1-methylethyl)biphenyl-2-yl]phosphane (XPHOS), bis(2-phenylphosphinophenyl) ether (DPEphos) or 4,5-bis(diphenylphosphino)-9,9-dimethylxanthene (Xantphos) [cf., for example, Hassan J. et al., Chem. Rev. 102, 1359-1469 (2002)].

The conversion (V)+(VI)→(I-A) is optionally carried out in the presence of a suitable base. Suitable bases for this conversion are the customary inorganic or organic bases. These preferably include alkali metal hydroxides, for example lithium hydroxide, sodium hydroxide or potassium hydroxide, alkali metal or alkaline earth metal carbonates such as lithium carbonate, sodium carbonate, potassium carbonate, calcium carbonate or caesium carbonate, alkali metal alkoxides such as sodium methoxide or potassium methoxide, sodium ethoxide or potassium ethoxide or sodium or potassium tert-butoxide, alkali metal hydrides such as sodium hydride or potassium hydride, amides such as sodium amide, lithium bis(trimethylsilyl)amide, sodium bis(trimethylsilyl)amide or potassium bis(trimethylsilyl)amide or lithium diisopropylamide, or organic amines such as triethylamine, N-methylmorpholine, N-methylpiperidine, N,N-diisopropylethylamine, pyridine, 1,5-diazabicyclo[4.3.0]non-5-ene (DBN), 1,8-diazabicyclo[5.4.0]undec-7-ene (DBU) or 1,4-diazabicyclo[2.2.2]octane (DABCO®) or potassium phosphate. Preference is given to using potassium phosphate.

The reaction (V)+(VI)→(I-A) is generally carried out in a temperature range from 0° C. to +200° C., preferably at from +100° C. to +150° C. The conversion can be carried out under atmospheric, elevated or reduced pressure (for example from 0.5 to 5 bar). In general, the reaction is carried out at atmospheric pressure.

Inert solvents for the process step (I-A)+(VIII)→(VI-A) or (I-A)+(VIII)→(VII-B) are, for example, halohydrocarbons such as dichloromethane, trichloromethane, tetrachloromethane, trichloroethylene or chlorobenzene, ethers such as diethyl ether, dioxane, tetrahydrofuran, glycol dimethyl ether or diethylene glycol dimethyl ether, hydrocarbons such as benzene, toluene, xylene, hexane, cyclohexane or mineral oil fractions, or other solvents such as acetone, methyl ethyl ketone, ethyl acetate, acetonitrile, N,N-dimethylformamide, N,N-dimethylacetamide, dimethyl sulphoxide, N,N′-dimethylpropyleneurea (DMPU), N-methylpyrrolidone (NMP) or pyridine. It is also possible to use mixtures of the solvents mentioned. Preference is given to using dimethylformamide or dimethyl sulphoxide.

Suitable bases for the process step (I-A)+(VIII)→(VII-A) or (I-A)+(VIII)→(VII-B) are the customary inorganic or organic bases. These preferably include alkali metal hydroxides, for example lithium hydroxide, sodium hydroxide or potassium hydroxide, alkali metal or alkaline earth metal carbonates such as lithium carbonate, sodium carbonate, potassium carbonate, calcium sodium iodide or potassium iodide, alkali metal alkoxides such as sodium methoxide or potassium methoxide, sodium ethoxide or potassium ethoxide or sodium or potassium tert-butoxide, alkali metal hydrides such as sodium hydride or potassium hydride, amides such as sodium amide, lithium bis(trimethylsilyl)amide or potassium bis(trimethylsilyl)amide or lithium diisopropylamide, or organic amines such as triethylamine, N-methylmorpholine, N-methylpiperidine, N,N-diisopropylethylamine, pyridine, 4-(N,N-dimethylamino)pyridine (DMAP), 1,5-diazabicyclo[4.3.0]non-5-ene (DBN), 1,8-diazabicyclo[5.4.0]undec-7-ene (DBU) or 1,4-diazabicyclo[2.2.2]octane (DABCO®). Preference is given to using potassium carbonate, caesium carbonate or sodium methoxide.

The reaction is generally carried out within a temperature range from 0° C. to +120° C., preferably at +20° C. to +80° C., optionally in a microwave. The reaction can be carried out under atmospheric, elevated or reduced pressure (for example from 0.5 to 5 bar).

Inert solvents for the process step (VI-A)→(I-B) or (VII-B)→(I-C) are, for example, alcohols such as methanol, ethanol, n-propanol, isopropanol, n-butanol or tert-butanol, and also dichloromethane, ethyl acetate, THF, dioxane, DMF, water, acetic acid, dilute hydrochloric acid or water. It is also possible to use mixtures of the solvents mentioned. Preference is given to using ethanol.

The reactions (VI-A)→(I-B) or (VII-B)→(I-C) are carried out in the presence of a suitable catalyst. Suitable catalysts are, for example, palladium/carbon, palladium(II) hydroxide/carbon, platinum(IV) oxide, platinum and Raney nickel. Preference is given to using Raney nickel or palladium/carbon.

The reaction is carried out generally within a temperature range from 0° C. to +120° C., preferably at +20° C. to +80° C.

The reaction is carried out in a hydrogen atmosphere at standard or elevated pressure (e.g. from 1.0 to 50 bar). Preferably, the reaction is carried out at standard hydrogen pressure.

Alternatively to hydrogen, it is also possible to employ other hydrogen sources such as cyclohexene, cyclohexadiene and ammonium formate.

The compounds of the formula (II) are known from the literature or can be prepared by reacting a compound of the formula (IX)

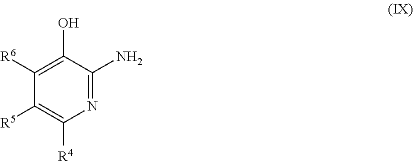

in which R⁴, R⁵ and R⁶ have the meaning given above,
in an inert solvent in the presence of a suitable base with a compound of the formula (X)

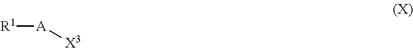

in which A and R¹ have the meaning given above and
X³ represents a suitable leaving group, in particular chlorine, bromine, iodine, mesylate, triflate or tosylate,
   to give a compound of the formula (XI)

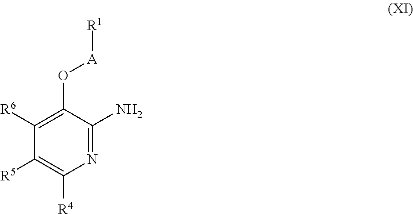

in which R¹, R⁴, R⁵ and R⁶ each have the meanings given above,
and then reacting this in an inert solvent with a compound of the formula (XII)

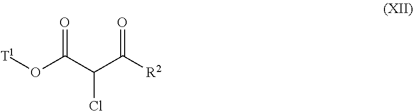

in which R² and T¹ are each as defined above.

The process described is illustrated in an exemplary manner by the scheme below (Scheme 3):

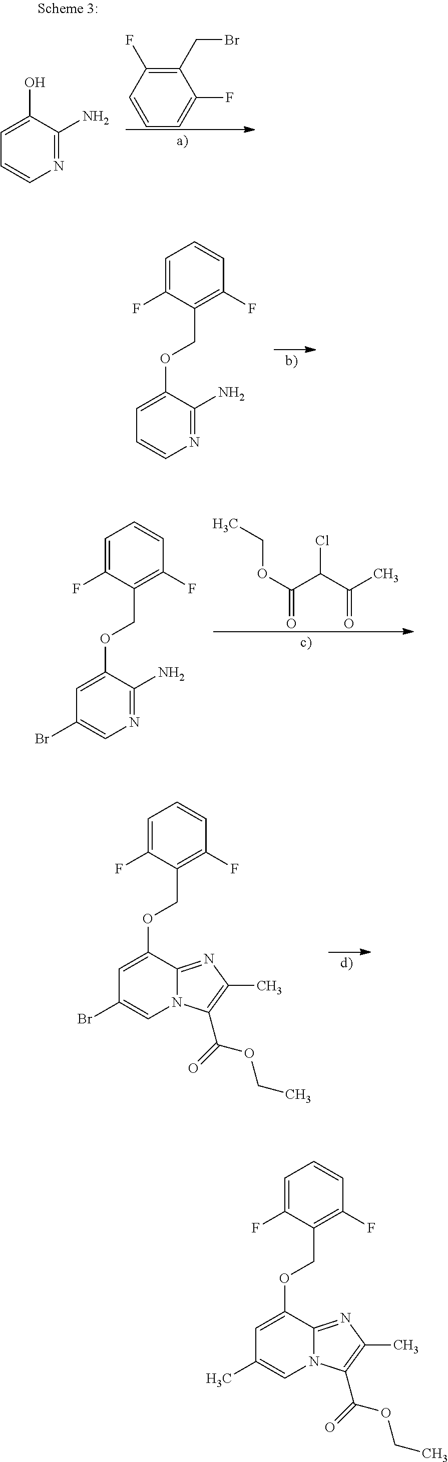

[a): i) sodium methoxide, methanol, RT; ii) DMSO, RT; b): Br₂, H₂SO₄/HOAc c): EtOH, molecular sieve, reflux; d): 1,1′-bis(diphenylphosphino)ferrocenepalladium(II) dichloride/dichloromethane, methylzinc chloride, THF, 100° C.].

The synthesis sequence shown can be modified such that the respective reaction steps are carried out in a different order. An example of such a modified synthesis sequence is shown in Scheme 4.

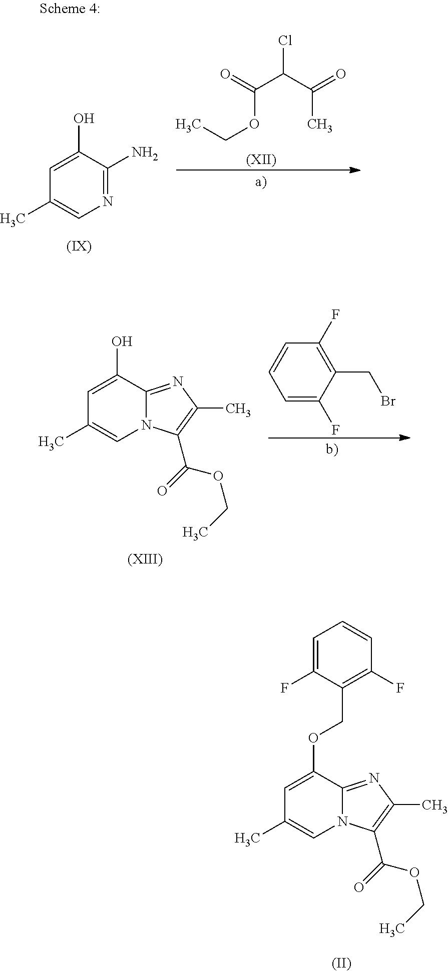

[a): EtOH, molecular sieve, reflux; b): b) caesium carbonate, DMF, 50° C.].

Inert solvents for the process step (IX)+(X)→(XI) are, for example, halohydrocarbons such as dichloromethane, trichloromethane, tetrachloromethane, trichloroethylene or chlorobenzene, ethers such as diethyl ether, dioxane, tetrahydrofuran, glycol dimethyl ether or diethylene glycol dimethyl ether, hydrocarbons such as benzene, toluene, xylene, hexane, cyclohexane or mineral oil fractions, alcohols such as methanol, ethanol, tert-butanol, or other solvents such as acetone, methyl ethyl ketone, ethyl acetate, acetonitrile, N,N-dimethylformamide, dimethyl sulphoxide, N,N′-dimethylpropyleneurea (DMPU), N-methylpyrrolidone (NMP) or pyridine. It is also possible to use mixtures of the solvents mentioned. Preference is given to using methanol, dimethylformamide or dimethyl sulphoxide.

Suitable bases for the process step (IX)+(X)→(XI) are the customary inorganic or organic bases. These preferably include alkali metal hydroxides, for example lithium hydroxide, sodium hydroxide or potassium hydroxide, alkali metal or alkaline earth metal carbonates such as lithium carbonate, sodium carbonate, potassium carbonate, calcium carbonate or caesium carbonate, optionally with addition of an alkali metal iodide, for example sodium iodide or potassium iodide, alkali metal alkoxides such as sodium methoxide or potassium methoxide, sodium ethoxide or potassium ethoxide or sodium or potassium tert-butoxide, alkali metal hydrides such as sodium hydride or potassium hydride, amides such as sodium amide, lithium bis(trimethylsilyl)amide or potassium bis(trimethylsilyl)amide or lithium diisopropylamide, or organic amines such as triethylamine, N-methylmorpholine, N-methylpiperidine, N,N-diisopropylethylamine, pyridine, 1,5-diazabicyclo[4.3.0]non-5-ene (DBN), 1,8-diazabicyclo[5.4.0]undec-7-ene (DBU) or 1,4-diazabicyclo[2.2.2]octane (DABCO®). Preference is given to using potassium carbonate, caesium carbonate or sodium methoxide.

The reaction is generally carried out within a temperature range from 0° C. to +120° C., preferably at +20° C. to +80° C., optionally in a microwave. The reaction can be carried out under atmospheric, elevated or reduced pressure (for example from 0.5 to 5 bar).

Inert solvents for the ring closure to give the imidazo[1,2-a]pyridine base skeleton (XI)+(XII)→(II) or (IX)+(XII)→(XIII) are the customary organic solvents. These preferably include alcohols such as methanol, ethanol, n-propanol, isopropanol, n-butanol, n-pentanol or tert-butanol, or ethers such as diethyl ether, tetrahydrofuran, 2-methyltetrahydrofuran, dioxane or glycol dimethyl ether, or other solvents such as acetone, dichloromethane, 1,2-dichloroethane, acetonitrile, dimethylformamide or dimethyl sulphoxide. It is also possible to use mixtures of the solvents mentioned. Preference is given to using ethanol.

The ring closure is generally carried out within a temperature range from +50° C. to +150° C., preferably at +50° C. to +100° C., optionally in a microwave.

The ring closure (XI)+(XII)→(II) or (IX)+(XII)→(XIII) is optionally carried out in the presence of dehydrating reaction additives, for example in the presence of molecular sieve (pore size 4 Å) or by means of a water separator. The reaction (XI)+(XII)→(II) or (IX)+(XII)→(XIII) is carried out using an excess of the reagent of the formula (XII), for example with 1 to 20 equivalents of the reagent (XII), optionally with addition of bases (for example sodium hydrogencarbonate), in which case the addition of this reagent can be carried out all at once or in several portions.

Further compounds of the invention can optionally also be prepared by conversions of functional groups of individual substituents, especially those listed for R³, proceeding from the compounds of the formula (I) obtained by above processes. These conversions are performed by customary methods known to those skilled in the art and include, for example, reactions such as nucleophilic and electrophilic substitutions, oxidations, reductions, hydrogenations, transition metal-catalysed coupling reactions, eliminations, alkylation, amination, esterification, ester hydrolysis, etherification, ether hydrolysis, formation of carbonamides, and introduction and removal of temporary protective groups.

The compounds of the invention have valuable pharmacological properties and can be used for the prevention and treatment of diseases in humans and animals. The compounds of the invention offer a further treatment alternative and thus enlarge the field of pharmacy.

The compounds of the invention bring about vasorelaxation and inhibition of platelet aggregation, and lead to a decrease in blood pressure and to a rise in coronary blood flow. These effects are mediated by a direct stimulation of soluble guanylate cyclase and an intracellular rise in cGMP. In addition, the compounds of the invention enhance the action of substances which increase the cGMP level, for example EDRF (endothelium-derived relaxing factor), NO donors, protoporphyrin IX, arachidonic acid or phenylhydrazine derivatives.

The compounds of the invention are suitable for the treatment and/or prophylaxis of cardiovascular, pulmonary, thromboembolic and fibrotic disorders.

Accordingly, the compounds according to the invention can be used in medicaments for the treatment and/or prophylaxis of cardiovascular disorders such as, for example, high blood pressure (hypertension), resistant hypertension, acute and chronic heart failure, coronary heart disease, stable and unstable angina pectoris, peripheral and cardiac vascular disorders, arrhythmias, atrial and ventricular arrhythmias and impaired conduction such as, for example, atrioventricular blocks degrees I-III (AB block supraventricular tachyarrhythmia, atrial fibrillation, atrial flutter, ventricular fibrillation, ventricular flutter, ventricular tachyarrhythmia, Torsade de pointes tachycardia, atrial and ventricular extrasystoles, AV-junctional extrasystoles, sick sinus syndrome, syncopes, AV-nodal re-entry tachycardia, Wolff-Parkinson-White syndrome, of acute coronary syndrome (ACS), autoimmune cardiac disorders (pericarditis, endocarditis, valvolitis, aortitis, cardiomyopathies), shock such as cardiogenic shock, septic shock and anaphylactic shock, aneurysms, boxer cardiomyopathy (premature ventricular contraction (PVC)), for the treatment and/or prophylaxis of thromboembolic disorders and ischaemias such as myocardial ischaemia, myocardial infarction, stroke, cardiac hypertrophy, transient and ischaemic attacks, preeclampsia, inflammatory cardiovascular disorders, spasms of the coronary arteries and peripheral arteries, oedema formation such as, for example, pulmonary oedema, cerebral oedema, renal oedema or oedema caused by heart failure, peripheral circulatory disturbances, reperfusion damage, arterial and venous thromboses, microalbuminuria, myocardial insufficiency, endothelial dysfunction, to prevent restenoses, for example after thrombolysis therapies, percutaneous transluminal angioplasties (PTA), transluminal coronary angioplasties (PTCA), heart transplants and bypass operations, and also micro- and macrovascular damage (vasculitis), increased levels of fibrinogen and of low-density lipoprotein (LDL) and increased concentrations of plasminogen activator inhibitor 1 (PAI-1), and also for the treatment and/or prophylaxis of erectile dysfunction and female sexual dysfunction.

In the context of the present invention, the term “heart failure” encompasses both acute and chronic manifestations of heart failure, and also more specific or related types of disease, such as acute decompensated heart failure, right heart failure, left heart failure, global failure, ischaemic cardiomyopathy, dilated cardiomyopathy, hypertrophic cardiomyopathy, idiopathic cardiomyopathy, congenital heart defects, heart failure associated with heart valve defects, mitral valve stenosis, mitral valve insufficiency, aortic valve stenosis, aortic valve insufficiency, tricuspid valve stenosis, tricuspid valve insufficiency, pulmonary valve stenosis, pulmonary valve insufficiency, combined heart valve defects, myocardial inflammation (myocarditis), chronic myocarditis, acute myocarditis, viral myocarditis, diabetic heart failure, alcoholic cardiomyopathy, cardiac storage disorders, diastolic heart failure and systolic heart failure and acute phases of worsening of existing chronic heart failure (worsening heart failure).

In addition, the compounds of the invention can also be used for the treatment and/or prophylaxis of arteriosclerosis, impaired lipid metabolism, hypolipoproteinaemias, dyslipidaemias, hypertriglyceridaemias, hyperlipidaemias, hypercholesterolaemias, abetelipoproteinaemia, sitosterolaemia, xanthomatosis, Tangier disease, adiposity, obesity and of combined hyperlipidaemias and metabolic syndrome.

The compounds of the invention can also be used for the treatment and/or prophylaxis of primary and secondary Raynaud's phenomenon, microcirculation impairments, claudication, peripheral and autonomic neuropathies, diabetic microangiopathies, diabetic retinopathy, diabetic ulcers on the extremities, gangrene, CREST syndrome, erythematosis, onychomycosis, rheumatic disorders and for promoting wound healing.

The compounds according to the invention are furthermore suitable for treating urological disorders such as, for example, benign prostate syndrome (BPS), benign prostate hyperplasia (BPH), benign prostate enlargement (BPE), bladder outlet obstruction (BOO), lower urinary tract syndromes (LUTS, including Feline Urological Syndrome (FUS)), disorders of the urogenital system including neurogenic over-active bladder (OAB) and (IC), incontinence (UI) such as, for example, mixed urinary incontinence, urge urinary incontinence, stress urinary incontinence or overflow urinary incontinence (MUI, UUI, SUI, OUI), pelvic pain, benign and malignant disorders of the organs of the male and female urogenital system.

The compounds of the invention are also suitable for the treatment and/or prophylaxis of kidney disorders, in particular of acute and chronic renal insufficiency and acute and chronic renal failure. In the context of the present invention, the term “renal insufficiency” encompasses both acute and chronic manifestations of renal insufficiency, and also underlying or related renal disorders such as renal hypoperfusion, intradialytic hypotension, obstructive uropathy, glomerulopathies, glomerulonephritis, acute glomerulonephritis, glomerulosclerosis, tubulointerstitial diseases, nephropathic disorders such as primary and congenital kidney disease, nephritis, immunological kidney disorders such as kidney transplant rejection and immunocomplex-induced kidney disorders, nephropathy induced by toxic substances, nephropathy induced by contrast agents, diabetic and non-diabetic nephropathy, pyelonephritis, renal cysts, nephrosclerosis, hypertensive nephrosclerosis and nephrotic syndrome which can be characterized diagnostically, for example by abnormally reduced creatinine and/or water excretion, abnormally elevated blood concentrations of urea, nitrogen, potassium and/or creatinine, altered activity of renal enzymes, for example glutamyl synthetase, altered urine osmolarity or urine volume, elevated microalbuminuria, macroalbuminuria, lesions on glomerulae and arterioles, tubular dilatation, hyperphosphataemia and/or need for dialysis. The present invention also encompasses the use of the compounds of the invention for the treatment and/or prophylaxis of sequelae of renal insufficiency, for example pulmonary oedema, heart failure, uraemia, anaemia, electrolyte disorders (for example hyperkalaemia, hyponatraemia) and disorders in bone and carbohydrate metabolism.

In addition, the compounds of the invention are also suitable for the treatment and/or prophylaxis of asthmatic disorders, pulmonary arterial hypertension (PAH) and other forms of pulmonary hypertension (PH) including left-heart disease-, HIV-, sickle cell anaemia-, thromboembolism (CTEPH)-, sarcoidosis-, COPD- or pulmonary fibrosis-associated pulmonary hypertension, chronic-obstructive pulmonary disease (COPD), acute respiratory distress syndrome (ARDS), acute lung injury (ALI), alpha-1-antitrypsin deficiency (AATD), pulmonary fibrosis, pulmonary emphysema (for example pulmonary emphysema induced by cigarette smoke) and cystic fibrosis (CF).

The compounds described in the present invention are also active compounds for control of central nervous system disorders characterized by disturbances of the NO/cGMP system. They are suitable in particular for improving perception, concentration, learning or memory after cognitive impairments like those occurring in particular in association with situations/diseases/syndromes such as mild cognitive impairment, age-associated learning and memory impairments, age-associated memory losses, vascular dementia, craniocerebral trauma, stroke, dementia occurring after strokes (post-stroke dementia), post-traumatic craniocerebral trauma, general concentration impairments, concentration impairments in children with learning and memory problems, Alzheimer's disease, Lewy body dementia, dementia with degeneration of the frontal lobes including Pick's syndrome, Parkinson's disease, progressive nuclear palsy, dementia with corticobasal degeneration, amyolateral sclerosis (ALS), Huntington's disease, demyelinization, multiple sclerosis, thalamic degeneration, Creutzfeldt-Jakob dementia, HIV dementia, schizophrenia with dementia or Korsakoff's psychosis. They are also suitable for the treatment and/or prophylaxis of central nervous system disorders such as states of anxiety, tension and depression, CNS-related sexual dysfunctions and sleep disturbances, and for controlling pathological disturbances of the intake of food, stimulants and addictive substances.

In addition, the compounds of the invention are also suitable for controlling cerebral blood flow and are effective agents for controlling migraine. They are also suitable for the prophylaxis and control of sequelae of cerebral infarct (Apoplexia cerebri) such as stroke, cerebral ischaemias and skull-brain trauma. The compounds according to the invention can likewise be used for controlling states of pain and tinnitus.

In addition, the compounds of the invention have anti-inflammatory action and can therefore be used as anti-inflammatory agents for the treatment and/or prophylaxis of sepsis (SIRS), multiple organ failure (MODS, MOF), inflammatory disorders of the kidney, chronic intestinal inflammations (IBD, Crohn's disease, UC), pancreatitis, peritonitis, rheumatoid disorders, inflammatory skin disorders and inflammatory eye disorders.

Furthermore, the compounds according to the invention can also be used for the treatment and/or prophylaxis of autoimmune diseases.

The compounds of the invention are also suitable for the treatment and/or prophylaxis of fibrotic disorders of the internal organs, for example the lung, the heart, the kidney, the bone marrow and in particular the liver, and also dermatological fibroses and fibrotic eye disorders. In the context of the present invention, the term fibrotic disorders includes in particular the following terms: hepatic fibrosis, cirrhosis of the liver, pulmonary fibrosis, endomyocardial fibrosis, nephropathy, glomerulonephritis, interstitial renal fibrosis, fibrotic damage resulting from diabetes, bone marrow fibrosis and similar fibrotic disorders, scleroderma, morphea, keloids, hypertrophic scarring (also following surgical procedures), naevi, diabetic retinopathy, proliferative vitroretinopathy and disorders of the connective tissue (for example sarcoidosis).

The compounds of the invention are also suitable for controlling postoperative scarring, for example as a result of glaucoma operations.

The compounds of the invention can also be used cosmetically for ageing and keratinizing skin.

Moreover, the compounds according to the invention are suitable for the treatment and/or prophylaxis of hepatitis, neoplasms, osteoporosis, glaucoma and gastroparesis.

The present invention further provides for the use of the compounds according to the invention for the treatment and/or prophylaxis of disorders, especially the disorders mentioned above.

The present invention further provides for the use of the compounds according to the invention for the treatment and/or prophylaxis of heart failure, angina pectoris, hypertension, pulmonary hypertension, ischaemias, vascular disorders, renal insufficiency, thromboembolic disorders, fibrotic disorders and arteriosclerosis.

The present invention further provides the compounds of the invention for use in a method for the treatment and/or prophylaxis of heart failure, angina pectoris, hypertension, pulmonary hypertension, ischaemias, vascular disorders, renal insufficiency, thromboembolic disorders, fibrotic disorders and arteriosclerosis.

The present invention further provides for the use of the compounds according to the invention for production of a medicament for the treatment and/or prophylaxis of disorders, especially the disorders mentioned above.

The present invention further provides for the use of the compounds according to the invention for production of a medicament for the treatment and/or prophylaxis of heart failure, angina pectoris, hypertension, pulmonary hypertension, ischaemias, vascular disorders, renal insufficiency, thromboembolic disorders, fibrotic disorders and arteriosclerosis.

The present invention further provides a method for the treatment and/or prophylaxis of disorders, in particular the disorders mentioned above, using an effective amount of at least one of the compounds of the invention.

The present invention further provides a method for the treatment and/or prophylaxis of heart failure, angina pectoris, hypertension, pulmonary hypertension, ischaemias, vascular disorders, renal insufficiency, thromboembolic disorders, fibrotic disorders and arteriosclerosis using an effective amount of at least one of the compounds of the invention.

The compounds according to the invention can be used alone or, if required, in combination with other active compounds. The present invention further provides medicaments comprising at least one of the compounds of the invention and one or more further active compounds, especially for the treatment and/or prophylaxis of the aforementioned disorders. Preferred examples of active compounds suitable for combinations include:
organic nitrates and NO donors, for example sodium nitroprusside, nitroglycerin, isosorbide mononitrate, isosorbide dinitrate, molsidomine or SIN-1, and inhaled NO;
compounds which inhibit the breakdown of cyclic guanosine monophosphate (cGMP), for example inhibitors of phosphodiesterases (PDE) 1, 2 and/or 5, especially PDE 5 inhibitors such as sildenafil, vardenafil and tadalafil;
antithrombotic agents, by way of example and with preference from the group of the platelet aggregation inhibitors, the anticoagulants or the profibrinolytic substances;
hypotensive active compounds, by way of example and with preference from the group of the calcium antagonists, angiotensin AII antagonists, ACE inhibitors, endothelin antagonists, renin inhibitors, alpha-receptor blockers, beta-receptor blockers, mineralocorticoid receptor antagonists, and the diuretics; and/or
active compounds altering lipid metabolism, for example and with preference from the group of the thyroid receptor agonists, cholesterol synthesis inhibitors such as, by way of example and preferably, HMG-CoA reductase inhibitors or squalene synthesis inhibitors, the ACAT inhibitors, CETP inhibitors, MTP inhibitors, PPAR-alpha, PPAR-gamma and/or PPAR-delta agonists, cholesterol absorption inhibitors, lipase inhibitors, polymeric bile acid adsorbents, bile acid reabsorption inhibitors and lipoprotein(a) antagonists.

Antithrombotic agents are preferably understood to mean compounds from the group of the platelet aggregation inhibitors, the anticoagulants or the profibrinolytic substances.

In a preferred embodiment of the invention, the compounds according to the invention are administered in combination with a platelet aggregation inhibitor, by way of example and with preference aspirin, clopidogrel, ticlopidine or dipyridamole.

In a preferred embodiment of the invention, the compounds of the invention are administered in combination with a thrombin inhibitor, by way of example and with preference ximelagatran, dabigatran, melagatran, bivalirudin or clexane.

In a preferred embodiment of the invention, the compounds according to the invention are administered in combination with a GPIIb/IIIa antagonist, by way of example and with preference tirofiban or abciximab.

In a preferred embodiment of the invention, the compounds of the invention are administered in combination with a factor Xa inhibitor, by way of example and with preference rivaroxaban (BAY 59-7939), DU-176b, apixaban, otamixaban, fidexaban, razaxaban, fondaparinux, idraparinux, PMD-3112, YM-150, KFA-1982, EMD-503982, MCM-17, MLN-1021, DX 9065a, DPC 906, JTV 803, SSR-126512 or SSR-128428.

In a preferred embodiment of the invention, the compounds according to the invention are administered in combination with heparin or with a low molecular weight (LMW) heparin derivative.

In a preferred embodiment of the invention, the compounds according to the invention are administered in combination with a vitamin K antagonist, by way of example and with preference coumarin.

Hypotensive agents are preferably understood to mean compounds from the group of the calcium antagonists, angiotensin AII antagonists, ACE inhibitors, endothelin antagonists, renin inhibitors, alpha-receptor blockers, beta-receptor blockers, mineralocorticoid receptor antagonists, and the diuretics.

In a preferred embodiment of the invention, the compounds according to the invention are administered in combination with a calcium antagonist, by way of example and with preference nifedipine, amlodipine, verapamil or diltiazem.

In a preferred embodiment of the invention, the compounds according to the invention are administered in combination with an alpha-1-receptor blocker, by way of example and with preference prazosin.

In a preferred embodiment of the invention, the compounds according to the invention are administered in combination with a beta-receptor blocker, by way of example and with preference propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, the compounds according to the invention are administered in combination with an angiotensin AII antagonist, by way of example and with preference losartan, candesartan, valsartan, telmisartan or embursatan.

In a preferred embodiment of the invention, the compounds according to the invention are administered in combination with an ACE inhibitor, by way of example and with preference enalapril, captopril, lisinopril, ramipril, delapril, fosinopril, quinopril, perindopril or trandopril.

In a preferred embodiment of the invention, the compounds according to the invention are administered in combination with an endothelin antagonist, by way of example and with preference bosentan, darusentan, ambrisentan or sitaxsentan.

In a preferred embodiment of the invention, the compounds according to the invention are administered in combination with a renin inhibitor, by way of example and with preference aliskiren, SPP-600 or SPP-800.

In a preferred embodiment of the invention, the compounds according to the invention are administered in combination with a mineralocorticoid receptor antagonist, by way of example and with preference spironolactone or eplerenone.

In a preferred embodiment of the invention, the compounds according to the invention are administered in combination with a loop diuretic, for example furosemide, torasemide, bumetanide and piretanide, with potassium-sparing diuretics, for example amiloride and triamterene, with aldosterone antagonists, for example spironolactone, potassium canrenoate and eplerenone, and also thiazide diuretics, for example hydrochlorothiazide, chlorthalidone, xipamide and indapamide.

Lipid metabolism modifiers are preferably understood to mean compounds from the group of the CETP inhibitors, thyroid receptor agonists, cholesterol synthesis inhibitors such as HMG-CoA reductase inhibitors or squalene synthesis inhibitors, the ACAT inhibitors, MTP inhibitors, PPAR-alpha, PPAR-gamma and/or PPAR-delta agonists, cholesterol absorption inhibitors, polymeric bile acid adsorbents, bile acid reabsorption inhibitors, lipase inhibitors and the lipoprotein(a) antagonists.

In a preferred embodiment of the invention, the compounds according to the invention are administered in combination with a CETP inhibitor, by way of example and with preference dalcetrapib, BAY 60-5521, anacetrapib or CETP vaccine (CETi-1).

In a preferred embodiment of the invention, the compounds according to the invention are administered in combination with a thyroid receptor agonist, by way of example and with preference D-thyroxine, 3,5,3′-triiodothyronine (T3), CGS 23425 or axitirome (CGS 26214).

In a preferred embodiment of the invention, the compounds according to the invention are administered in combination with an HMG-CoA reductase inhibitor from the class of statins, by way of example and with preference lovastatin, simvastatin, pravastatin, fluvastatin, atorvastatin, rosuvastatin or pitavastatin.

In a preferred embodiment of the invention, the compounds according to the invention are administered in combination with a squalene synthesis inhibitor, by way of example and with preference BMS-188494 or TAK-475.

In a preferred embodiment of the invention, the compounds according to the invention are administered in combination with an ACAT inhibitor, by way of example and with preference avasimibe, melinamide, pactimibe, eflucimibe or SMP-797.

In a preferred embodiment of the invention, the compounds according to the invention are administered in combination with an MTP inhibitor, by way of example and with preference implitapide, BMS-201038, R-103757 or JTT-130.

In a preferred embodiment of the invention, the compounds according to the invention are administered in combination with a PPAR-gamma agonist, by way of example and with preference pioglitazone or rosiglitazone.

In a preferred embodiment of the invention, the compounds according to the invention are administered in combination with a PPAR-delta agonist, by way of example and with preference GW 501516 or BAY 68-5042.

In a preferred embodiment of the invention, the compounds according to the invention are administered in combination with a cholesterol absorption inhibitor, by way of example and with preference ezetimibe, tiqueside or pamaqueside.

In a preferred embodiment of the invention, the compounds according to the invention are administered in combination with a lipase inhibitor, by way of example and with preference orlistat.

In a preferred embodiment of the invention, the compounds according to the invention are administered in combination with a polymeric bile acid adsorber, by way of example and with preference cholestyramine, colestipol, colesolvam, CholestaGel or colestimide.

In a preferred embodiment of the invention, the compounds according to the invention are administered in combination with a bile acid reabsorption inhibitor, by way of example and with preference ASBT (=IBAT) inhibitors, for example AZD-7806, S-8921, AK-105, BARI-1741, SC-435 or SC-635.

In a preferred embodiment of the invention, the compounds according to the invention are administered in combination with a lipoprotein(a) antagonist, by way of example and with preference gemcabene calcium (CI-1027) or nicotinic acid.

The present invention further provides medicaments which comprise at least one compound according to the invention, typically together with one or more inert, non-toxic, pharmaceutically suitable auxiliaries, and for the use thereof for the aforementioned purposes.

The compounds according to the invention can act systemically and/or locally. For this purpose, they can be administered in a suitable manner, for example by the oral, parenteral, pulmonal, nasal, sublingual, lingual, buccal, rectal, dermal, transdermal, conjunctival or otic route, or as an implant or stent.

The compounds according to the invention can be administered in administration forms suitable for these administration routes.

Suitable administration forms for oral administration are those which work according to the prior art and release the compounds according to the invention rapidly and/or in a modified manner and which contain the compounds according to the invention in crystalline and/or amorphized and/or dissolved form, for example tablets (uncoated or coated tablets, for example with gastric juice-resistant or retarded-dissolution or insoluble coatings which control the release of the compound of the invention), tablets or films/oblates which disintegrate rapidly in the oral cavity, films/lyophilizates, capsules (for example hard or soft gelatin capsules), sugar-coated tablets, granules, pellets, powders, emulsions, suspensions, aerosols or solutions.

Parenteral administration can be accomplished with avoidance of a resorption step (for example by an intravenous, intraarterial, intracardiac, intraspinal or intralumbar route) or with inclusion of a resorption (for example by an intramuscular, subcutaneous, intracutaneous, percutaneous or intraperitoneal route). Administration forms suitable for parenteral administration include preparations for injection and infusion in the form of solutions, suspensions, emulsions, lyophilizates or sterile powders.

For the other administration routes, suitable examples are inhalable medicament forms (including powder inhalers, nebulizers), nasal drops, solutions or sprays, tablets, films/oblates or capsules for lingual, sublingual or buccal administration, suppositories, ear or eye preparations, vaginal capsules, aqueous suspensions (lotions, shaking mixtures), lipophilic suspensions, ointments, creams, transdermal therapeutic systems (e.g. patches), milk, pastes, foams, sprinkling powders, implants or stents.

Preference is given to oral or parenteral administration, especially oral administration.

The compounds according to the invention can be converted to the administration forms mentioned. This can be accomplished in a manner known per se by mixing with inert, non-toxic, pharmaceutically suitable auxiliaries. These auxiliaries include carriers (for example microcrystalline cellulose, lactose, mannitol), solvents (e.g. liquid polyethylene glycols), emulsifiers and dispersing or wetting agents (for example sodium dodecylsulphate, polyoxysorbitan oleate), binders (for example polyvinylpyrrolidone), synthetic and natural polymers (for example albumin), stabilizers (e.g. antioxidants, for example ascorbic acid), colorants (e.g. inorganic pigments, for example iron oxides) and flavour and/or odour correctants.

In general, it has been found to be advantageous in the case of parenteral administration to administer amounts of about 0.001 to 1 mg/kg, preferably about 0.01 to 0.5 mg/kg, of body weight to achieve effective results. In the case of oral administration, the dose is about 0.001 to 2 mg/kg, preferably about 0.001 to 1 mg/kg, of body weight.

It may nevertheless be necessary in some cases to deviate from the stated amounts, specifically as a function of body weight, route of administration, individual response to the active compound, nature of the preparation and time or interval over which administration takes place. Thus in some cases it may be sufficient to manage with less than the abovementioned minimum amount, while in other cases the upper limit mentioned must be exceeded. In the case of administration of greater amounts, it may be advisable to divide them into several individual doses over the day.

The working examples which follow illustrate the invention. The invention is not restricted to the examples.

Unless stated otherwise, the percentages in the tests and examples which follow are percentages by weight; parts are parts by weight. Solvent ratios, dilution ratios and concentration data for the liquid/liquid solutions are based in each case on volume.

### A. EXAMPLES

### Abbreviations and Acronyms

aq. aqueous solution
calc. calculated
br. broad signal (NMR coupling pattern)
CAS No. Chemical Abstracts Service number
δ shift in the NMR spectrum (stated in ppm)
d doublet (NMR coupling pattern)
TLC thin-layer chromatography
DCI direct chemical ionization (in MS)
DMAP 4-N,N-dimethylaminopyridine
DMF dimethylformamide
DMSO dimethyl sulphoxide
EDCI N-[3-(dimethylamino)propyl]-N′-ethylcarbodiimide
eq. equivalent(s)
ESI electrospray ionization (in MS)
Et ethyl
h hour(s)
HATU N-[(dimethylamino)(3H-[1,2,3]triazolo[4,5-b]-pyridin-3-yloxy)methylene]-N-methylmethanaminium hexafluorophosphate
HOBT 1H-benzotriazol-1-ol
HPLC high-pressure, high-performance liquid chromatography
HRMS high-resolution mass spectrometry
ID internal diameter
conc. concentrated
LC-MS liquid chromatography-coupled mass spectrometry
LiHMDS lithium hexamethyldisilazide
m multiplet
Me methyl
min minute(s)
MS mass spectrometry
NMR nuclear magnetic resonance spectrometry
PDA photodiode array detector
Pd₂dba₃ tris(dibenzylideneacetone)dipalladium
Ph phenyl
q quartet (NMR coupling pattern)
quint. quintet (NMR coupling pattern)
R_{F} retention factor (in thin-layer chromatography)
RT room temperature
Rₜ retention time (in HPLC)
s singlet (NMR coupling pattern)
t triplet (NMR coupling pattern)
THF tetrahydrofuran
TBTU (benzotriazol-1-yloxy)bisdimethylaminomethylium fluoroborate
UPLC-MS ultra-pressure liquid chromatography-coupled mass spectrometry
UV ultraviolet spectrometry
v/v ratio by volume (of a solution)
Xantphos 4,5-bis(diphenylphosphino)-9,9-dimethylxanthene
XPHOS dicyclohexyl(2′,4′,6′-triisopropylbiphenyl-2-yl)phosphine

The multiplicities of proton signals in ¹H NMR spectra reported in the paragraphs which follow represent the signal form observed in each case and do not take account of any higher-order signal phenomena. In all ¹H NMR spectra data, the chemical shifts δ are stated in ppm.

Additionally, the starting materials, intermediates and working examples may be present as hydrates. There was no quantitative determination of the water content. In certain cases, the hydrates may affect the ¹H NMR spectrum and possibly shift and/or significantly broaden the water signal in the ¹H NMR.

Unless stated otherwise, the percentages in the tests and examples which follow are percentages by weight; parts are parts by weight. Solvent ratios, dilution ratios and concentration data for the liquid/liquid solutions are based in each case on volume.

When compounds of the invention are purified by preparative HPLC by the above-described methods in which the mobile phases contain additives, for example trifluoroacetic acid, formic acid or ammonia, the compounds of the invention may be obtained in salt form, for example as trifluoroacetate, formate or ammonium salt, if the compounds of the invention contain a sufficiently basic or acidic functionality. Such a salt can be converted to the corresponding free base or acid by various methods known to the person skilled in the art.

In the case of the synthesis intermediates and working examples of the invention described hereinafter, any compound specified in the form of a salt of the corresponding base or acid is generally a salt of unknown exact stoichiometric composition, as obtained by the respective preparation and/or purification process. Unless specified in more detail, additions to names and structural formulae, such as “hydrochloride”, “trifluoroacetate”, “sodium salt” or “x HCl”, “x CF₃COOH”, “x Na⁺” should not therefore be understood in a stoichiometric sense in the case of such salts, but have merely descriptive character with regard to the salt-forming components present therein.

This applies correspondingly if synthesis intermediates or working examples or salts thereof were obtained in the form of solvates, for example hydrates, of unknown stoichiometric composition (if they are of a defined type) by the preparation and/or purification processes described.

### LC/MS and HPLC Methods:

### Method 1 (LC-MS):

Instrument: Waters ACQUITY SQD UPLC system; column: Waters Acquity UPLC HSS T3 1.8μ 50×1 mm; mobile phase A: 1 l of water+0.25 ml of 99% strength formic acid, mobile phase B: 1 l of acetonitrile+0.25 ml of 99% strength formic acid; gradient: 0.0 min 90% A→1.2 min 5% A→2.0 min 5% A; oven: 50° C.; flow rate: 0.40 ml/min; UV detection: 210-400 nm.

### Method 2 (LC-MS):

Instrument: Micromass Quattro Premier with Waters UPLC Acquity; column: Thermo Hypersil GOLD 1.9μ 50 mm×1 mm; mobile phase A: 1 l of water+0.5 ml of 50% strength formic acid, mobile phase B: 1 l of acetonitrile+0.5 ml of 50% strength formic acid; gradient: 0.0 min 90% A→0.1 min 90% A→1.5 min 10% A→2.2 min 10% A; flow rate: 0.33 ml/min; oven: 50° C.; UV detection: 210 nm.

### Method 3 (DCI-MS):

Instrument: DSQ II; Thermo Fisher-Scientific; DCI with NH₃, flow rate: 1.1 ml/min; source temperature: 200° C.; ionization energy 70 eV; heat DCI filament to 800° C.; mass range 80-900.

### Method 4 (LCMS):

Instrument: Waters ACQUITY SQD UPLC system; column: Waters Acquity UPLC HSS T3 1.8μ 30×2 mm; mobile phase A: 1 l of water+0.25 ml of 99% strength formic acid, mobile phase B: 1 l of acetonitrile+0.25 ml of 99% strength formic acid; gradient: 0.0 min 90% A→1.2 min 5% A→2.0 min 5% A oven: 50° C.; flow rate: 0.60 ml/min; UV detection: 208-400 nm.

### Method 5 (LC-MS):

Instrument: Acquity UPLC coupled with Quattro Micro mass spectrometer; column: Acquity UPLC BEH C18 (50 mm×2.1 mm ID, 1.7 μm packing diameter); mobile phase A: 10 mM aqueous ammonium bicarbonate solution (adjusted with ammonia to a pH of 10), mobile phase B: acetonitrile; gradient: 0.0 min 97% A, 3% B, flow rate 1 ml/min; 1.5 min 100% B, flow rate 1 ml/min; 1.9 min 100% B, flow rate 1 ml/min; 2.0 min 97% A, 3% B, flow rate 0.05 ml/min; column temperature: 40° C.; UV detection: from 210 nm to 350 nm; MS conditions: ionization mode: alternating scans positive and negative electrospray (ES+/ES−); scan range: 100 to 1000 AMU.

### Method 6 (LC-MS):

Instrument: Acquity UPLC coupled with Quattro Micro mass spectrometer; column: Acquity UPLC BEH C18 (50 mm×2.1 mm ID, 1.7 μm packing diameter); mobile phase A: 0.1% formic acid in water, mobile phase B: 0.1% formic acid in acetonitrile; gradient: 0.0 min 97% A, 3% B, flow rate 1 ml/min; 1.5 min 100% B, flow rate 1 ml/min; 1.9 min 100% B, flow rate 1 ml/min; 2.0 min 97% A, 3% B, flow rate 0.05 ml/min; column temperature: 40° C.; UV detection: from 210 nm to 350 nm; MS conditions: ionization mode: alternating scans positive and negative electrospray (ES+/ES−); scan range: 100 to 1000 AMU.

### Method 7 (LC-MS):

Instrument: Waters 2690, PDA detector Waters 2996 coupled with Quattro Micro mass MS detector; column: Waters SunFire C18 3.5 μm, 2.1×50 mm; mobile phase A: 10 mM aqueous ammonium bicarbonate solution (adjusted with ammonia to a pH of 10), mobile phase B: acetonitrile; gradient: 0.0 min 95% A, 5% B, flow rate 0.5 ml/min; 3.0 min 95% A, 5% B, flow rate 0.5 ml/min; 17.50 min 5% A, 95% B, flow rate 0.5 ml/min; 19.00 min 5% A, 95% B, flow rate 0.5 ml/min; 19.50 min 95% A, 5% B, flow rate 0.5 ml/min; 20.00 min 95% A, 5% B, flow rate 0.5 ml/min; column temperature: 30° C.; UV detection: from 210 nm to 400 nm; MS conditions: ionization mode: scans positive and negative electrospray (ES+/ES−); scan range: 130 to 1100 AMU.

### Method 8 (LC-MS):

Instrument: Waters 2690, PDA detector Waters 2996 coupled with Quattro Micro mass MS detector; column: Waters SunFire C18 3.5 μm, 2.1×50 mm; mobile phase A: 0.1% formic acid in water, mobile phase B: 0.1% formic acid in acetonitrile; gradient: 0.0 min 95% A, 5% B, flow rate 0.5 ml/min; 3.0 min 95% A, 5% B, flow rate 0.5 ml/min; 17.50 min 5% A, 95% B, flow rate 0.5 ml/min; 19.00 min 5% A, 95% B, flow rate 0.5 ml/min; 19.50 min 95% A, 5% B, flow rate 0.5 ml/min; 20.00 min 95% A, 5% B, flow rate 0.5 ml/min; column temperature: 30° C.; UV detection: from 210 nm to 400 nm; MS conditions: ionization mode: scans positive and negative electrospray (ES+/ES−); scan range: 130 to 1100 AMU.

Methode 9 (prep. HPLC):

Instrument: Waters 2690, PDA detector Waters 2996 coupled with Quattro Micro mass MS detector; column: XBridge Prep. MS C18 OBD (150 mm×30 mm ID 5 μm particle size) at room temperature; mobile phase A: 10 mM NH₄HCO₃, adjusted with ammonia to a pH of 10, mobile phase B: acetonitrile; gradient: 0.0 min 97% A, 3% B; 1.0 min 97% A, 3% B; 30 min 0% A, 100% B; 35 min 0% A, 100% B, flow rate 50 ml/min; column temperature: 30° C.; UV detection: from 210 nm to 400 nm; MS conditions: ionization mode: scans positive and negative electrospray (ES+/ES−); scan range: 100 to 1000 AMU.

### Starting Materials and Intermediates

### Example 1A

### 3-[(2,6-Difluorobenzyl)oxy]pyridine-2-amine

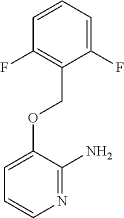 [SMILES: Nc1ncccc1OCc1c(F)cccc1F]

At RT, 51 g of sodium methoxide (953 mmol, 1.05 equivalents) were initially charged in 1000 ml of methanol, 100 g of 2-amino-3-hydroxypyridine (908 mmol, 1 equivalent) were added and the mixture was stirred at RT for 15 min. The reaction mixture was concentrated under reduced pressure, the residue was taken up in 2500 ml of DMSO and 197 g of 2,6-difluorobenzyl bromide (953 mmol, 1.05 equivalents) were added. After 4 h at RT, the reaction mixture was added to 20 l of water, the mixture was stirred for a further 15 min and the solid was filtered off. The solid was washed with 1 l of water and 100 ml of isopropanol and 500 ml of petroleum ether and dried under high vacuum. This gave 171 g of the title compound (78% of theory).

¹H-NMR (400 MHz, DMSO-d₆): δ=5.10 (s, 2H), 5.52 (br. s, 2H), 6.52 (dd, 1H), 7.16-7.21 (m, 3H), 7.49-7.56 (m, 2H).

### Example 2A

### 5-Bromo-3-[(2,6-difluorobenzyl)oxy]pyridine-2-amine

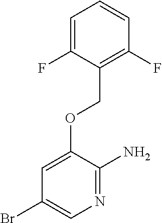 [SMILES: Nc1ncc(Br)cc1OCc1c(F)cccc1F]

32.6 g of 3-[(2,6-difluorobenzyl)oxy]pyridine-2-amine (Example 1A; 138 mmol, 1 equivalent) were suspended in 552 ml of 10% strength sulphuric acid, and the mixture was cooled to 0° C. 8.5 ml of bromine (165 mmol, 1.2 equivalents) were dissolved in 85 ml of acetic acid and then, over 90 min, added dropwise to the reaction solution, cooled with ice. After the addition had ended, the mixture was stirred at 0° C. for 90 min and then diluted with 600 ml of ethyl acetate, and the aqueous phase was separated off. The aqueous phase was extracted with ethyl acetate. The organic phases were combined, washed with saturated aqueous sodium bicarbonate solution, dried and concentrated. The residue was dissolved in dichloromethane and chromatographed on silica gel (petroleum ether/ethyl acetate gradient as mobile phase). This gave 24 g (55% of theory) of the title compound.

LC-MS (Method 1): Rₜ=0.96 min

MS (ESpos): m/z=315.1/317.1 (M+H)⁺

¹H-NMR (400 MHz, DMSO-d₆): δ=5.14 (s, 2H), 5.83 (br. s, 2H), 7.20 (t, 2H), 7.42 (d, 1H), 7.54 (q, 1H), 7.62 (d, 1H).

### Example 3A

### Ethyl 6-bromo-8-[(2,6-difluorobenzyl)oxy]-2-methylimidazo[1,2-a]pyridine-3-carboxylate

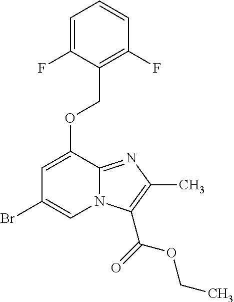 [SMILES: CCOC(=O)c1c(C)nc2c(OCc3c(F)cccc3F)cc(Br)cn12]

16 g of powdered molecular sieve 3 Å and 52.7 ml of ethyl 2-chloroacetoacetate (380.8 mmol, 5 equivalents) were added to 24 g of 5-bromo-3-[(2,6-difluorobenzyl)oxy]pyridine-2-amine (Example 2A; 76.2 mmol; 1 equivalent) in 400 ml of ethanol, and the mixture was heated at reflux overnight. 8 g of molecular sieve were added and the mixture was heated at reflux for a further 24 h. The reaction mixture was concentrated under reduced pressure, and the residue was taken up in dichloromethane and chromatographed on silica gel (mobile phase: dichloromethane/methanol 20:1). The product-containing fractions were concentrated and the residue was stirred with 100 ml of diethyl ether for 30 min. The solid was then filtered off, washed with a little diethyl ether and dried. This gave 15 g (45% of theory) of the title compound.

LC-MS (Method 2): Rₜ=1.43 min

MS (ESpos): m/z=414.9/416.8 (M+H)⁺

¹H-NMR (400 MHz, DMSO-d₆): δ=1.36 (t, 3H), 2.54 (s, 3H; hidden by DMSO signal), 4.37 (q, 2H), 5.36 (s, 2H), 7.25 (t, 2H), 7.42 (d, 1H), 7.61 (q, 1H), 9.00 (d, 1H).

### Example 4A

### Ethyl 8-[(2,6-difluorobenzyl)oxy]-2,6-dimethylimidazo[1,2-a]pyridine-3-carboxylate

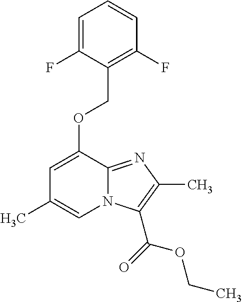 [SMILES: CCOC(=O)c1c(C)nc2c(OCc3c(F)cccc3F)cc(C)cn12]

### Method 1:

600 mg (1.4 mmol, 1 equivalent) of ethyl 6-bromo-8-[(2,6-difluorobenzyl)oxy]-2-methylimidazo[1,2-a]pyridine-3-carboxylate (Example 3A) and 230 mg of 1,1′-bis(diphenylphosphino)ferrocenepalladium(II) dichloride/dichloromethane complex (0.282 mmol, 20 mol %) were dissolved in 25 ml of THF, and 0.88 ml (1.76 mmol, 1.2 equivalents) of a 2 M solution of methylzinc chloride in THF was added. In a microwave, the reaction mixture was heated at 100° C. for 40 min. The reaction mixture was filtered through Celite and then concentrated under reduced pressure. The residue was chromatographed (Biotage Isolera Four; cyclohexane:ethyl acetate). This gave 225 mg (38% of theory) of the title compound.

### Method 2:

20.00 g (85.38 mmol) of ethyl 8-hydroxy-2,6-dimethylimidazo[1,2-a]pyridine-3-carboxylate from Example 9A, 19.44 g (93.91 mmol) of 2,6-difluorobenzyl bromide and 61.20 g (187.83 mmol) of caesium carbonate in 1.18 l of DMF were stirred at 60° C. for 5 h. The reaction mixture was then added to 6.4 l of 10% strength aqueous sodium chloride solution and then twice extracted with ethyl acetate. The combined organic phases were washed with 854 ml of 10% strength aqueous sodium chloride solution, dried, concentrated and dried at RT under high vacuum overnight. This gave 28.2 g (92% of theory; purity: 90%) of the title compound.

LC-MS (Method 1): Rₜ=1.05 min

MS (ESpos): m/z=361.1 (M+H)⁺

¹H-NMR (400 MHz, DMSO-d₆): δ=1.38 (t, 3H), 2.36 (s, 3H), 4.35 (q, 2H), 5.30 (s, 2H), 7.10 (s, 1H), 7.23 (t, 2H), 7.59 (q, 1H), 8.70 (s, 1H).

### Example 5A

### 8-[(2,6-Difluorobenzyl)oxy]-2,6-dimethylimidazo[1,2-a]pyridine-3-carboxylic acid

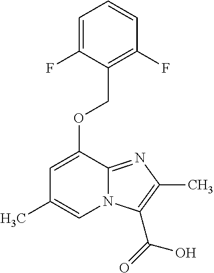

220 mg (0.524 mmol, 1 equivalent) of ethyl 8-[(2,6-difluorobenzyl)oxy]-2,6-dimethylimidazo[1,2-a]pyridine-3-carboxylate (Example 4A) were dissolved in 7 ml of THF/methanol (1:1), 2.6 ml of 1 N aqueous lithium hydroxide solution (2.6 mmol, 5 equivalents) were added and the mixture was stirred at RT for 16 h. The mixture was concentrated under reduced pressure and the residue was acidified with 1N aqueous hydrochloric acid and stirred for 15 min. The solid was filtered off, washed with water and dried under reduced pressure. This gave 120 mg of the title compound (60% of theory).

LC-MS (Method 1): Rₜ=0.68 min

MS (ESpos): m/z=333.1 (M+H)⁺

¹H-NMR (400 MHz, DMSO-d₆): δ=2.34 (s, 3H), 5.28 (s, 2H), 7.09 (s, 1H), 7.23 (t, 2H), 7.58 (q, 1H), 8.76 (s, 1H), 13.1 (br. s, 1H).

### Example 6A

### 3-(Benzyloxy)-5-bromopyridine-2-amine

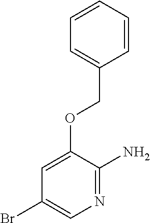 [SMILES: Nc1ncc(Br)cc1OCc1ccccc1]

The target compound is known from the literature and described:

1) Palmer, A. M. et al. J Med. Chem. 2007, 50, 6240-6264.

### 2) ALTANA WO2005/58325

### 3) ALTANA WO2005/90358

4) Cui, J. T. et al. J Med. Chem. 2011, 54, 6342-6363

Further preparation method:

200 g (1 mol) of 2-amino-3-benzyloxypyridine were initially charged in 4 l of dichloromethane, and at 0° C. a solution of 62 ml (1.2 mol) of bromine in 620 ml of dichloromethane was added over 30 min. After the addition had ended, the reaction solution was stirred at 0° C. for 60 min. About 4 l of saturated aqueous sodium bicarbonate solution were then added to the mixture. The organic phase was removed and concentrated. The residue was purified by silica gel column chromatography (petroleum ether:ethyl acetate 6:4) and the product fractions were concentrated. This gave 214 g (77% of theory) of the title compound.

LC-MS (Method 1): Rₜ=0.92 min

MS (ESpos): m/z=279 (M+H)⁺

¹H-NMR (400 MHz, DMSO-d₆): δ=5.16 (s, 2H), 5.94-6.00 (m, 2H), 7.26-7.29 (m, 1H), 7.31-7.36 (m, 1H), 7.37-7.43 (m, 2H), 7.47-7.52 (m, 2H), 7.57-7.59 (m, 1H).

### Example 7A

### Ethyl 8-(benzyloxy)-6-bromo-2-methylimidazo[1,2-a]pyridine-3-carboxylate

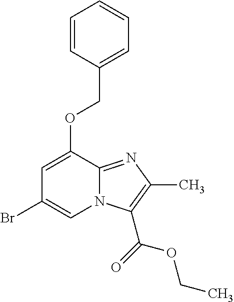 [SMILES: CCOC(=O)c1c(C)nc2c(OCc3ccccc3)cc(Br)cn12]

Under argon, 200 g (0.72 mol) of 3-(benzyloxy)-5-bromopyridine-2-amine from Example 6A, 590 g (3.58 mol) of ethyl 2-chloroacetoacetate and 436 g of 3 A molecular sieve were suspended in 6 l of ethanol, and the suspension was stirred at reflux for 72 h. The reaction mixture was filtered off through silica gel and concentrated. The residue was purified by silica gel chromatography (petroleum ether:ethyl acetate 9:1, then 6:4) and the product fractions were concentrated. This gave 221 g (79% of theory) of the target compound.

LC-MS (Method 4): Rₜ=1.31 min

MS (ESpos): m/z=389 (M+H)⁺

¹H-NMR (400 MHz, DMSO-d₆): δ=1.36 (t, 3H), 2.58 (s, 3H), 4.32-4.41 (m, 2H), 5.33 (s, 2H), 7.28-7.32 (m, 1H), 7.36-7.47 (m, 3H), 7.49-7.54 (m, 2H), 8.98 (d, 1H).

### Example 8A

### Ethyl 8-(benzyloxy)-2,6-dimethylimidazo[1,2-a]pyridine-3-carboxylate

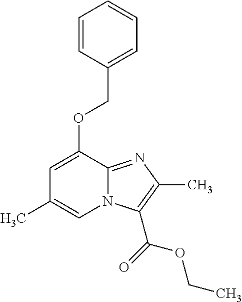 [SMILES: CCOC(=O)c1c(C)nc2c(OCc3ccccc3)cc(C)cn12]

Under argon, 105 g (270 mmol) of ethyl 8-(benzyloxy)-6-bromo-2-methylimidazo[1,2-a]pyridine-3-carboxylate from Example 7A were suspended in 4.2 l of 1,4-dioxane, and 135.4 g (539 mmol, purity 50%) of trimethylboroxine, 31.2 g (27 mmol) of tetrakis(triphenylphosphine)palladium(0) and 78.3 g (566 mmol) of potassium carbonate were added in succession and the mixture was stirred under reflux for 8 h. The reaction mixture was cooled to RT and, using silica gel, freed from the precipitate by filtration, and the filtrate was concentrated. The residue was dissolved in dichloromethane and purified by silica gel chromatography (dichloromethane:ethyl acetate=9:1). This gave 74 g (84.6% of theory; purity 100%) of the target compound.

LC-MS (Method 4): Rₜ=1.06 min; diastereomeric purity:

MS (ESpos): m/z=325 (M+H)⁺

¹H-NMR (400 MHz, DMSO-d₆): δ=1.35 (t, 3H), 2.34 (br. s, 3H), 2.56 (s, 3H), 4.31-4.38 (m, 2H), 5.28 (br. s, 2H), 6.99-7.01 (m, 1H), 7.35-7.47 (m, 3H), 7.49-7.54 (m, 2H), 8.68-8.70 (m, 1H).

### Example 9A

### Ethyl 8-hydroxy-2,6-dimethylimidazo[1,2-a]pyridine-3-carboxylate

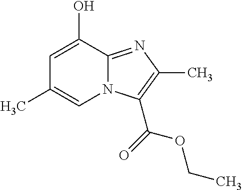 [SMILES: CCOC(=O)c1c(C)nc2c(O)cc(C)cn12]

74 g (228 mmol) of ethyl 8-(benzyloxy)-2,6-dimethylimidazo[1,2-a]pyridine-3-carboxylate from Example 8A were initially charged in 1254 ml of dichloromethane and 251 ml of ethanol, and 20.1 g of 10% palladium on activated carbon (moist with water, 50%) were added under argon. The reaction mixture was hydrogenated at RT and under standard pressure overnight. The reaction mixture was filtered off through silica gel and concentrated. The crude product was purified by silica gel chromatography (dichloromethane:methanol=95:5). This gave 50.4 g (94% of theory) of the target compound.

DCI-MS: (Method 3) (ESpos): m/z=235.2 (M+H)⁺

¹H-NMR (400 MHz, DMSO-d₆): δ=1.35 (t, 3H), 2.27 (s, 3H), 2.58 (s, 3H), 4.30-4.38 (m, 2H), 6.65 (d, 1H), 8.59 (s, 1H), 10.57 (br. s, 1H).

### Example 10A

### 8-[(2,6-Difluorobenzyl)oxy]-2,6-dimethylimidazo[1,2-a]pyridine

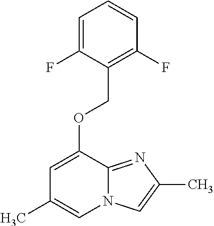

10.0 g (30.09 mmol) of 8-[(2,6-difluorobenzyl)oxy]-2,6-dimethylimidazo[1,2-a]pyridine-3-carboxylic acid from Example 5A were initially charged in 228 ml of dioxane, 25.1 ml of 6 N aqueous hydrochloric acid solution were added and the mixture was stirred at 100° C. for 2 h. After cooling, dioxane was removed under reduced pressure and the aqueous residue was adjusted to pH 8 using 2 N aqueous sodium hydroxide solution. The solid obtained was filtered off, washed with water and dried under high vacuum. This gave 8.97 g of the target compound (97% of theory, purity 94%).

LC-MS (Method 1): Rₜ=0.70 min

MS (ESpos): m/z=289 (M+H)⁺

¹H-NMR (400 MHz, DMSO-d₆): δ=2.22-2.30 (m, 6H); 5.27 (s, 2H); 6.67 (s, 1H); 7.21 (t, 2H); 7.53-7.63 (m, 2H); 7.89 (s, 1H).

### Example 11A

### 3-Bromo-8-[(2,6-difluorobenzyl)oxy]-2,6-dimethylimidazo[1,2-a]pyridine

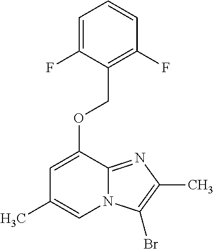

Under argon and with exclusion of light, 3.865 g (13.41 mmol) of 8-[(2,6-difluorobenzyl)oxy]-2,6-dimethylimidazo[1,2-a]pyridine from Example 10A were initially charged in 42 ml of ethanol, 2.625 g (14.75 mmol) of N-bromosuccinimide were added and the mixture was stirred at room temperature for 4 h. The reaction mixture was concentrated. The residue was stirred with about 100 ml of water, and the resulting suspension was then stirred at RT for 30 min. The precipitate formed was filtered off, washed with water and dried under high vacuum. This gave 4.48 g of the target compound (91% of theory, purity 100%).

LC-MS (Method 1): Rₜ=0.93 min

MS (ESpos): m/z=267 (M+H)⁺

¹H-NMR (400 MHz, DMSO-d₆): δ=2.28 (s, 3H), 2.33 (s, 3H); 5.30 (s, 2H); 6.89 (s, 1H); 7.22 (t, 2H); 7.53-7.63 (m, 1H); 7.75 (s, 1H).

### Example 12A

### 8-[(2,6-Difluorobenzyl)oxy]-2,6-dimethylimidazo[1,2-a]pyridine-3-carboxamide

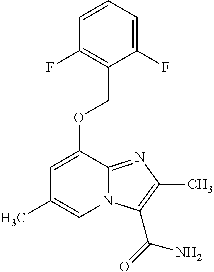

7.0 g (21.07 mmol) of 8-[(2,6-difluorobenzyl)oxy]-2,6-dimethylimidazo[1,2-a]pyridine-3-carboxylic acid from Example 5A were initially charged in 403 ml of dichloromethane, 6.06 g (31.60 mmol) of 1-(3-dimethylaminopropyl)-3-ethylcarbodiimide hydrochloride and 4.27 g (31.60 mmol) of 1-hydroxy-1H-benzotriazole hydrate were added and the mixture was stirred at room temperature for 10 min. Subsequently, 5.63 g (105.32 mmol) of ammonium chloride and 25.68 ml (147.5 mmol) of N,N-diisopropylethylamine were added and the mixture was stirred at room temperature overnight. Water was added to the reaction mixture, and the solid present was filtered off, then stirred with water at 50° C. for 30 min, filtered off again and washed with water. This gave 4.59 g (65% of theory) of the title compound. The combined filtrate fractions (dichloromethane/water) were separated into the phases. The dichloromethane phase was washed in each case once with saturated aqueous sodium bicarbonate solution and saturated aqueous sodium chloride solution. The organic phase was dried over sodium sulphate, filtered and concentrated under reduced pressure. The residue was stirred with a little acetonitrile and filtered off. This gave a further 1.29 g (17% of theory; purity 93%) of the title compound.

LC-MS (Method 1): Rₜ=0.64 min

MS (ESpos): m/z=332 (M+H)⁺

¹H-NMR (400 MHz, DMSO-d₆): δ=2.31 (s, 3H), 2.50 (s, 3H; hidden under DMSO signal), 5.28 (s, 2H), 6.92 (s, 1H), 7.22 (t, 2H), 7.35 (br. s, 2H), 7.53-7.63 (m, 1H); 8.62 (s, 1H).

### Example 13A

### 8-[(2,6-Difluorobenzyl)oxy]-2,6-dimethylimidazo[1,2-a]pyridine-3-carbonitrile

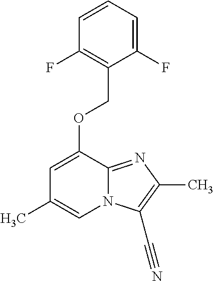

5.7 g (17.20 mol) of 8-[(2,6-difluorobenzyl)oxy]-2,6-dimethylimidazo[1,2-a]pyridine-3-carboxamide Example 12A were initially charged in 77 ml of THF, and 3.56 ml (44.0 mmol) of pyridine were added. At RT, 6.22 ml (44.0 mmol) of trifluoroacetic anhydride were then added dropwise, and the reaction mixture was stirred at RT for 3 h. After the reaction had ended, the mixture was added to water and extracted three times with ethyl acetate. The combined organic phases were washed once with saturated aqueous sodium bicarbonate solution, once with 1 N aqueous hydrochloric acid and once with saturated sodium chloride solution, dried over sodium sulphate and concentrated under reduced pressure. The residue was dried under reduced pressure overnight. This gave 5.47 g (90% of theory) of the title compound.

LC-MS (Method 1): Rₜ=1.12 min

MS (ESpos): m/z=314 (M+H)⁺

¹H-NMR (400 MHz, DMSO-d₆): δ=2.37 (s, 3H), 2.41 (s, 3H), 5.31 (s, 2H), 7.12 (s, 1H), 7.23 (t, 2H), 7.54-7.63 (m, 1H), 8.09 (s, 1H).

### Example 14A

### 8-[(2,6-Difluorobenzyl)oxy]-2,6-dimethylimidazo[1,2-a]pyridine-3-carboximidamide

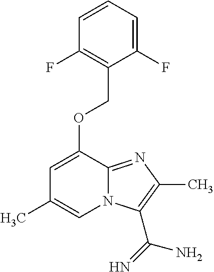

Under argon, 2.26 g (44.03 mmol, 2.52 equivalents) of ammonium chloride were initially charged in 69 ml of toluene, and the mixture was cooled to 0° C. At this temperature, 22.02 ml of a 2 molar solution of trimethylaluminium in toluene (44.04 mmol, 2.52 equivalents) were added, and the mixture was stirred at RT for 2 h. In another flask, 5.47 g of 8-[(2,6-difluorobenzyl)oxy]-2,6-dimethylimidazo[1,2-a]pyridine-3-carbonitrile from Example 13A (17.46 mmol, 1 equivalent) were initially charged in 58 ml of toluene, 34.3 ml of the solution prepared beforehand were added at RT and the mixture was stirred at 110° C. for 1 h. This procedure was repeated eight times. The mixture was then cooled, silica gel and a 1:1 mixture of dichloromethane/methanol were added at RT and the mixture was stirred at RT for 30 min. The silica gel was filtered off over a frit. The silica gel was washed with methanol and the filtrate was concentrated under reduced pressure. The residue was purified by silica gel chromatography (mobile phase: dichloromethane; dichloromethane:methanol=10:2). This gave 1.28 g (22% of theory) of the title compound.

LC-MS (Method 1): Rₜ=0.60 min

MS (ESpos): m/z=331.3 (M+H)⁺

¹H-NMR (400 MHz, DMSO-d₆): δ=2.35 (s, 3H), 2.43 (s, 3H), 5.31 (s, 2H), 7.06 (s, 1H), 7.24 (t, 2H), 7.54-7.65 (m, 1H), 8.02 (s, 1H), 9.25 (br. s, 3H).

LC-MS (Method 1): Rₜ=0.60 min

MS (ESpos): m/z=331.3 (M+H)⁺

¹H-NMR (400 MHz, DMSO-d₆): δ=2.35 (s, 3H), 2.43 (s, 3H), 5.31 (s, 2H), 7.06 (s, 1H), 7.24 (t, 2H), 7.54-7.65 (m, 1H), 8.02 (s, 1H), 9.25 (br. s, 3H).

### Example 15A

### 8-[(2,6-Difluorobenzyl)oxy]-2,6-dimethylimidazo[1,2-a]pyridine-3-carboximidohydrazide

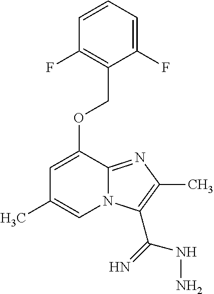

600 mg (1.82 mmol) of 8-[(2,6-difluorobenzyl)oxy]-2,6-dimethylimidazo[1,2-a]pyridine-3-carboximidamide from Example 14A were initially charged in ethanol (15 ml), and 2.025 ml (14.53 mmol) of triethylamine and then 220 μl (3.63 mmol) of hydrazine hydrate (80%) were added. The mixture was stirred at 50° C. overnight and then concentrated under reduced pressure. This gave 681 mg of crude product.

LC-MS (Method 1): Rₜ=0.55 min

MS (ESpos): m/z=346.2 (M+H)⁺

### Example 16A

### 2-Methyl-2-nitropropyl trifluoromethanesulphonate

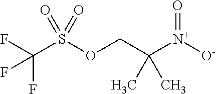

1.0 g (8.40 mmol) of 2-methyl-2-nitropropan-1-ol was initially charged in 20 ml of dichloromethane, 1.0 ml (12.59 mmol) of pyridine was added, the mixture was cooled to 0° C. and 1.85 ml (10.91 mmol) of trifluoromethanesulphonic anhydride was added slowly. The mixture was then stirred at 0° C. for 1 h. The course of the reaction was monitored by TLC (cyclohexane/ethyl acetate 7/3, staining reagent: potassium permanganate stain). The reaction solution was washed in each case once with water and saturated aqueous sodium chloride solution. The organic phase was dried over sodium sulphate and filtered and the filtrate was concentrated. This gave 2.18 g of the target compound (99% of theory). The target compound was stored at −18° C. and used without further purification.

MS (Method 3):

MS (ESpos): m/z=269 (M+NH₄)⁺

¹H NMR (400 MHz, DMSO-d₆) δ=1.64 (s, 6H), 5.13 (s, 2H).

### Example 17A

### 5-{8-[(2,6-Difluorobenzyl)oxy]-2,6-dimethylimidazo[1,2-a]pyridin-3-yl}-1-(2-methyl-2-nitropropyl)pyridin-2(1H)-one

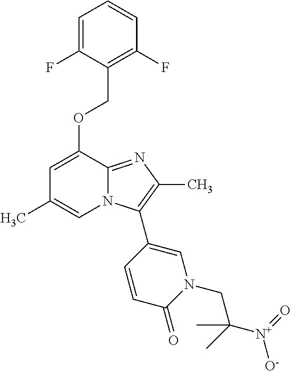 [SMILES: Cc1cc(OCc2c(F)cccc2F)c2nc(C)c(-c3ccc(=O)n(CC(C)(C)[N+](=O)[O-])c3)n2c1]

29.3 mg (0.12 mmol) of 2-methyl-2-nitropropyl trifluoromethanesulphonate Example 16A and then 103.8 mg (0.32 mmol) of caesium carbonate were added to a solution of 50 mg (0.10 mmol) of 5-{8-[(2,6-difluorobenzyl)oxy]-2,6-dimethylimidazo[1,2-a]pyridin-3-yl}pyridin-2(1H)-one Example 7 in 5 ml of dioxane. The reaction mixture was stirred at room temperature for 15 h. After the reaction had ended, the solvent was evaporated under reduced pressure and the residue was partitioned between 10 ml of dichloromethane and 10 ml of water. The aqueous phase was separated off and dried by lyophilization, and the residue was dissolved in 3 ml of methanol. The mother liquor was decanted off and concentrated under reduced pressure and the residue was purified by flash chromatography using a silica gel cartridge (mobile phase:dichloromethane-methanol 100:1 to 10:1), which gave 70 mg (44% yield, purity 32%) of the target compound.

LC-MS (Method 6): Rₜ=0.90 min; m/z=483 (M+H)⁺

### Example 18A

### 8-[(2,6-Difluorobenzyl)oxy]-2,6-dimethyl-3-{6-[(2-nitropropan-2-yl)oxy]pyridin-3-yl}imidazo[1,2-a]pyridine

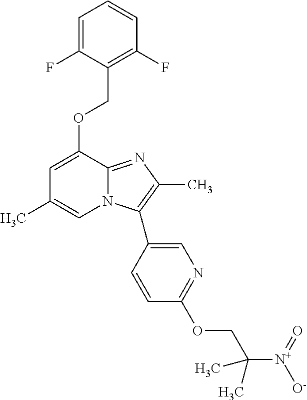 [SMILES: Cc1cc(OCc2c(F)cccc2F)c2nc(C)c(-c3ccc(OCC(C)(C)[N+](=O)[O-])nc3)n2c1]

21.7 mg (0.087 mmol) of 2-methyl-2-nitropropyl trifluoromethanesulphonate Example 16A and then 32.6 mg (0.236 mmol) of potassium carbonate were added to a solution of 30 mg (0.079 mmol) of 5-{8-[(2,6-difluorobenzyl)oxy]-2,6-dimethylimidazo[1,2-a]pyridin-3-yl}pyridin-2(1H)-one Example 7 in 3 ml of dimethylformamide. The reaction mixture was stirred at room temperature for 3 h and then partitioned between dichloromethane (20 ml) and water (10 ml). The phases were separated and the organic phase was concentrated under reduced pressure. The residue was purified by flash chromatography using a silica gel cartridge (mobile phase:dichloromethane-methanol 100:1 to 10:1), which gave 10 mg (25% yield, purity 93%) of the target compound.

LC-MS (Method 6): Rₜ=1.08 min; m/z=483.36 (M+H)⁺

### Example 19A

### 4-{8-[(2,6-Difluorobenzyl)oxy]-2,6-dimethylimidazo[1,2-a]pyridin-3-yl}-1-(2-methyl-2-nitropropyl)pyridin-2(1H)-one and

### Example 20A

### 8-[(2,6-Difluorobenzyl)oxy]-2,6-dimethyl-3-[2-(2-methyl-2-nitropropoxy)pyridin-4-yl]imidazo[1,2-a]pyridine

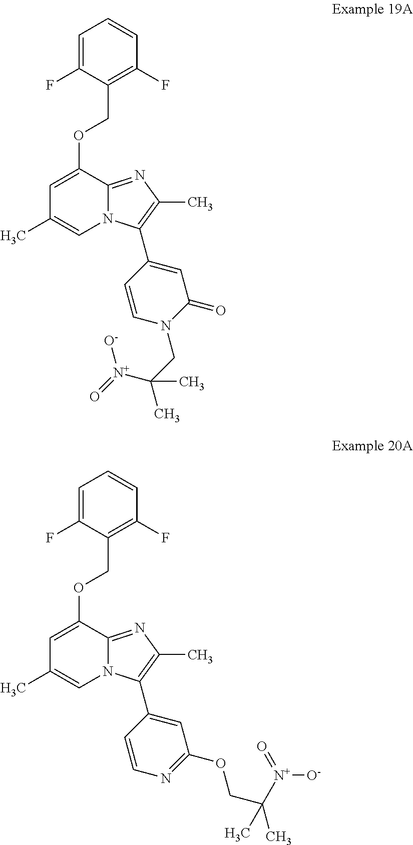 [SMILES: Cc1cc(OCc2c(F)cccc2F)c2nc(C)c(-c3ccn(CC(C)(C)[N+](=O)[O-])c(=O)c3)n2c1 ; Cc1cc(OCc2c(F)cccc2F)c2nc(C)c(-c3ccnc(OCC(C)(C)[N+](=O)[O-])c3)n2c1]

205 mg (0.629 mmol) of caesium carbonate and then 57.9 mg (0.231 mmol) of 2-methyl-2-nitropropyl trifluoromethanesulphonate Example 16A were added to a solution of 80 mg (0.210 mmol) of 4-{8-[(2,6-difluorobenzyl)oxy]-2,6-dimethylimidazo[1,2-a]pyridin-3-yl}pyridin-2(1H)-one Example 8 in 5 ml of dioxane. The resulting suspension was stirred at room temperature for 15 h, the solvent was removed under reduced pressure and the residue was partitioned between dichloromethane (20 ml) and water (10 ml). The phases were separated and the organic phase was concentrated under reduced pressure. The residue was purified by flash chromatography using a silica gel cartridge (mobile phase: cyclohexane-ethyl acetate 10:1 to 1:1), which gave 55 mg (50% yield, purity 92%) of Example 19A and 30 mg (29% yield, purity 98%) of target compound 20A.

### Example 19A

LC-MS (Method 6): Rₜ=0.84 min; m/z=483.41 (M+H)⁺

### Example 20A

LC-MS (Method 6): Rₜ=1.01 min; m/z=483.42 (M+H)⁺

### Example 21A

1H-Benzotriazol-1-yl{8-[(2,6-difluorobenzyl)oxy]-2,6-dimethylimidazo[1,2-a]pyridin-3-yl}methanone

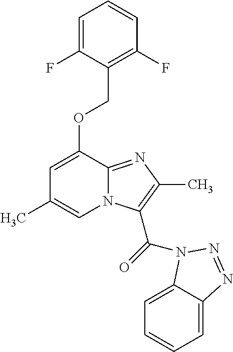 [SMILES: Cc1cc(OCc2c(F)cccc2F)c2nc(C)c(C(=O)n3nnc4ccccc43)n2c1]

A solution of 1.5 g (4.5 mmol) of 8-[(2,6-difluorobenzyl)oxy]-2,6-dimethylimidazo[1,2-a]pyridine-3-carboxylic acid Example 5A in 10 ml of undiluted thionyl chloride was stirred at 100° C. for 1 h. The solvent was removed under reduced pressure and the residue was suspended in 20 ml of dry dichloromethane. 476 mg (4.0 mmol) of 1H-1,2,3-benzotriazole were added, followed by the slow addition of 0.67 ml (4.8 mmol) of triethylamine. The reaction mixture was stirred at room temperature for 16 h, 0.1 M aqueous hydrochloric acid (5 ml) was then added and stirring was continued for a further 5 min. The organic phase was washed with water (20 ml), separated off, dried with a phase separation cartridge and concentrated under reduced pressure, which gave 1.5 g (86% of theory) of the target compound.

LC MS (Method 6): Rₜ=1.28 min; m/z=434.29 (M+H)+

¹H-NMR (300 MHz, DMSO-d₆): δ [ppm]=8.63 (s, 1H), 8.38 (d, 1H), 8.29 (d, 1H), 7.90 (t, 1H), 7.66-7.78 (m, 2H), 7.35 (t, 3H), 5.47 (s, 2H), 2.59 (s, 3H), 2.49 (s, 3H), 2.32 (s, 2H).

### Example 22A

### 1-{8-[(2,6-Difluorobenzyl)oxy]-2,6-dimethylimidazo[1,2-a]pyridin-3-yl}-5-methylhex-4-ene-1,3-dione

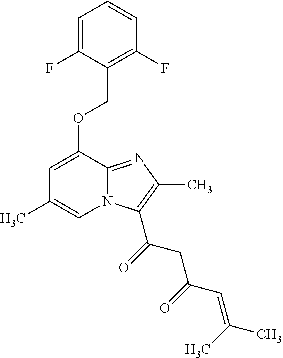 [SMILES: CC(C)=CC(=O)CC(=O)c1c(C)nc2c(OCc3c(F)cccc3F)cc(C)cn12]

1.4 ml (12.5 mmol) of 4-methylpent-3-en-2-one (CAS: 141-79-7) followed by 2.8 ml (16.6 mmol) of diisopropylethylamine were added to a suspension of 1.8 g (4.1 mmol) of 1H-benzotriazol-1-yl{8-[(2,6-difluorobenzyl)oxy]-2,6-dimethylimidazo[1,2-a]pyridin-3-yl}methanone Example 21A and 3.2 g (12.5 mmol) of magnesium bromide-diethyl ether complex in 20 ml of dichloromethane. The resulting mixture was stirred at room temperature overnight. 0.1 M aqueous hydrochloric acid (10 ml) was added, and stirring was continued for a further 5 min. The aqueous phase was extracted with dichloromethane (2×30 ml). The combined organic extracts were dried with a phase separation cartridge and concentrated under reduced pressure. The residue was purified by flash chromatography using a silica gel cartridge (mobile phase: dichloromethane/methanol 100:1 to 10:1), which gave 1.05 g (41% yield, purity 67%) of the target compound, which was used in the next step without further purification.

LC-MS (Method 7): Rₜ=1.38 min; m/z=413.37 (M+H)⁺

### Example 23A

### 5-Amino-1-{8-[(2,6-difluorobenzyl)oxy]-2,6-dimethylimidazo[1,2-a]pyridin-3-yl}-5-methylhexane-1,3-dione

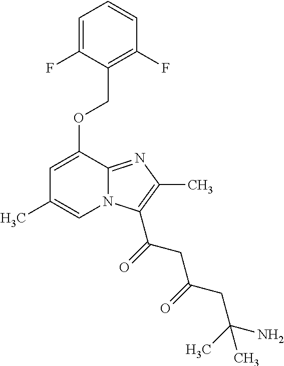 [SMILES: Cc1cc(OCc2c(F)cccc2F)c2nc(C)c(C(=O)CC(=O)CC(C)(C)N)n2c1]

2.3 g (29.0 mmol) of ammonium bicarbonate were added to a solution of 800 mg (1.3 mmol, purity 67%) of 1-{8-[(2,6-difluorobenzyl)oxy]-2,6-dimethylimidazo[1,2-a]pyridin-3-yl}-5-methylhex-4-ene-1,3-dione Example 22A in 15 ml of absolute ethanol. The mixture was heated to 80° C. and stirred for 15 min. The content was cooled to room temperature and stirred for a further 15 h. The reaction mixture was filtered and the mother liquor was concentrated under reduced pressure, which gave 1 g (33% yield, purity 28%) of the Example 23A. The crude product was used in the next step without further purification.

LC-MS (Method 6): Rₜ=0.85 min; m/z=430.37 (M+H)⁺

### Example 24A

### 5-{8-[(2,6-Difluorobenzyl)oxy]-2,6-dimethylimidazo[1,2-a]pyridin-3-yl}-1,2-dihydro-3H-1,2,4-triazol-3-one

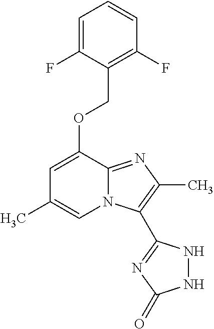

99 mg (0.612 mmol) of 1,1′-carbonyldiimidazole were added to a solution of 176 mg (0.51 mmol) of 8-[(2,6-difluorobenzyl)oxy]-2,6-dimethylimidazo[1,2-a]pyridine-3-carboximidohydrazide Example 15A in 4.5 ml of 1,4-dioxane. The reaction mixture was heated in a microwave at 90° C. for 20 minutes. The resulting precipitate was filtered off and dried under reduced pressure overnight, which gave 159 mg (82%, purity 97%) of the target compound.

LC-MS (Method 5): Rₜ=0.67 min; m/z=372.30 (M+H)⁺

¹H-NMR (500 MHz, DMSO-d₆): δ [ppm]=2.40 (s, 3H), 2.50 (s, 3H), 3.64 (s, 2H), 6.99 (s, 1H), 7.31 (m, 2H), 7.57-7.87 (m, 1H), 8.37 (s, 1H), 11.76 (br. s, 1H), 11.94 (s, 1H).

### Example 25A

### 8-[(2,6-Difluorobenzyl)oxy]-2,6-dimethyl-3-[3-(2-methyl-2-nitropropoxy)-1H-1,2,4-triazol-5-yl]imidazo[1,2-a]pyridine

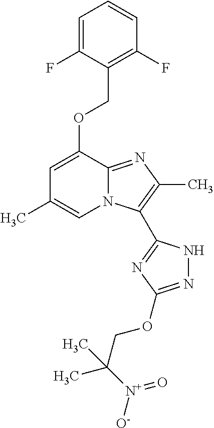

130 mg (0.399 mmol) of caesium carbonate and 100 mg (0.399 mmol) of 2-methyl-2-nitropropyl trifluoromethanesulphonate Example 16A were added to a solution of 153 mg (0.399 mmol) of 5-{8-[(2,6-difluorobenzyl)oxy]-2,6-dimethylimidazo[1,2-a]pyridin-3-yl}-1,2-dihydro-3H-1,2,4-triazol-3-one Example 24A in 2 ml of N,N-dimethylformamide. The reaction mixture was heated in a microwave at 100° C. for 20 minutes. After concentration under reduced pressure, the residue was purified by flash chromatography using a silica gel cartridge (mobile phase:dichloromethane:methanol 100:1 to 10:1). This gave 68 mg (33%, purity 91%) of the target compound.

LC-MS (Method 5): Rₜ=1.14 min; m/z=473.36 (M+H)⁺

¹H-NMR (300 MHz, DMSO-d₆): δ [ppm]=1.68 (s, 6H), 2.72 (s, 3H), 2.88 (s, 3H), 4.84 (s, 2H), 5.29 (s, 2H), 7.23 (m, 2H), 7.49-7.69 (m, 1H), 7.94 (s, 2H), 8.78 (s, 1H).

### WORKING EXAMPLES

### Example 1

### 1-(2-Amino-2-methylpropyl)-5-{8-[(2,6-difluorobenzyl)oxy]-2,6-dimethylimidazo[1,2-a]pyridin-3-yl}pyridin-2(1H)-one

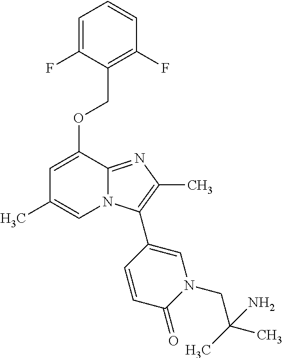 [SMILES: Cc1cc(OCc2c(F)cccc2F)c2nc(C)c(-c3ccc(=O)n(CC(C)(C)N)c3)n2c1]

1 ml of a suspension of Raney nickel in water was added to a solution of 70 mg (0.046 mmol, purity 32%) of 5-{8-[(2,6-difluorobenzyl)oxy]-2,6-dimethylimidazo[1,2-a]pyridin-3-yl}-1-(2-methyl-2-nitropropyl)pyridin-2(1H)-one Example 17A in 10 ml of absolute ethanol. The resulting mixture was hydrogenated at room temperature at 1 bar for 15 h. The reaction mixture was filtered through Celite and the mother liquor was concentrated under reduced pressure. The residue was purified by preparative HPLC chromatography (Method 9), which gave 3.3 mg (15% yield, purity 98%) of the target compound.

LC-MS (Method 7): Rₜ=11.77 min; m/z=453.54 (M+H)⁺

¹H-NMR (500 MHz, DMSO-d₆): δ [ppm]=1.05 (s, 6H), 2.25 (s, 3H), 2.26 (s, 3H), 3.90 (s, 2H), 5.28 (s, 2H), 6.54 (d, 1H), 6.75 (s, 1H), 7.24 (t, 2H), 7.51 (dd, 1H), 7.59 (quint., 1H), 7.76 (s, 1H), 7.88 (d, 1H).

¹³C-NMR (125 MHz, DMSO-d₆): δ [ppm]=13.8, 18.3, 28.9, 55.5, 57.2, 58.6, 105.4, 106.1, 112.0, 115.3, 118.3, 120.0, 121.3, 132.3, 136.9, 139.4, 141.1, 141.9, 146.4, 161.4, 161.4.

### Example 2

### 1-[(5-{8-[(2,6-Difluorobenzyl)oxy]-2,6-dimethylimidazo[1,2-a]pyridin-3-yl}pyridin-2-yl)oxy]-2-methylpropan-2-amine

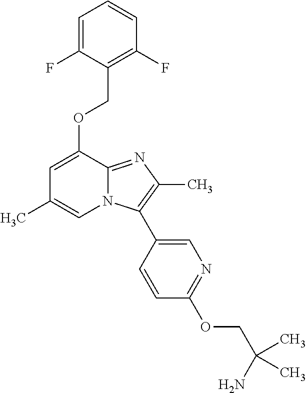 [SMILES: Cc1cc(OCc2c(F)cccc2F)c2nc(C)c(-c3ccc(OCC(C)(C)N)nc3)n2c1]

0.5 ml of a suspension of Raney nickel in water was added to a solution of 10 mg (0.021 mmol) of 8-[(2,6-difluorobenzyl)oxy]-2,6-dimethyl-3-{6-[(2-nitropropan-2-yl)oxy]pyridin-3-yl}imidazo[1,2-a]pyridine Example 18A in 5 ml of absolute ethanol. The reaction mixture was hydrogenated at room temperature at 1 bar for 15 h, the content was filtered through Celite and the mother liquor was concentrated to dryness under reduced pressure, which gave 8.0 mg (83% yield, purity 97%) of the target compound.

LC-MS (Method 8): Rₜ=6.75 min; m/z=453.16 (M+H)⁺

¹H-NMR (600 MHz, DMSO-d₆): δ [ppm]=1.12 (s, 6H), 2.25 (s, 6H), 4.03 (s, 2H), 5.29 (s, 2H), 6.77 (d, 1H), 7.02 (d, 1H), 7.20-7.29 (m, 2H), 7.56-7.61 (m, 1H), 7.62 (s, 1H), 7.83 (dd, 1H), 8.24 (d, 1H).

¹³C-NMR (150 MHz, DMSO-d₆): δ [ppm]=13.5, 18.0, 27.2, 49.0, 58.1, 75.5, 106.0, 111.1, 112.0, 114.4, 118.4, 121.1, 131.9, 139.5, 140.2, 145.9, 147.5, 161.2, 163.0.

### Example 3

### 1-(2-Amino-2-methylpropyl)-4-{8-[(2,6-difluorobenzyl)oxy]-2,6-dimethylimidazo[1,2-a]pyridin-3-yl}pyridin-2(1H)-one

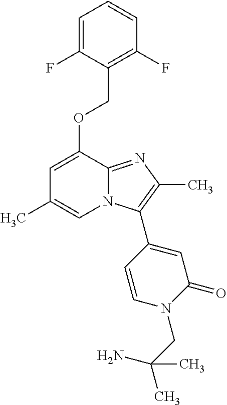 [SMILES: Cc1cc(OCc2c(F)cccc2F)c2nc(C)c(-c3ccn(CC(C)(C)N)c(=O)c3)n2c1]

1 ml of a suspension of Raney nickel in water was added to a solution of 55 mg (0.114 mmol) of 4-{8-[(2,6-difluorobenzyl)oxy]-2,6-dimethylimidazo[1,2-a]pyridin-3-yl}-1-(2-methyl-2-nitropropyl)pyridin-2(1H)-one Example 19A in 10 ml of absolute ethanol. The mixture was subsequently hydrogenated at 1 bar at room temperature for 15 h and then filtered through Celite. The mother liquor was concentrated under reduced pressure, which gave 18 mg (52% yield, purity 96%) of the target compound.

LC-MS (Method 7): Rₜ=11.11 min; m/z=453.57 (M+H)⁺

¹H-NMR (500 MHz, DMSO-d₆): δ [ppm]=1.05 (s, 6H), 2.30 (s, 3H), 2.34 (s, 3H), 3.87 (s, 2H), 5.29 (s, 2H), 6.36 (dd, 1H), 6.45 (d, 1H), 6.85 (s, 1H), 7.24 (t, 2H), 7.59 (quint., 1H), 7.80 (d, 1H), 7.86 (s, 1H).

¹³C-NMR (125 MHz, DMSO-d₆): δ [ppm]=14.3, 18.1, 28.5, 51.7, 57.3, 58.2, 104.1, 106.7, 111.9, 112.2, 115.4, 117.3, 119.2, 122.3, 132.4, 137.9, 140.4, 140.9, 141.2, 146.2, 161.7, 162.2.

### Example 4

### 1-[(4-{8-[(2,6-Difluorobenzyl)oxy]-2,6-dimethylimidazo[1,2-a]pyridin-3-yl}pyridin-2-yl)oxy]-2-methylpropan-2-amine

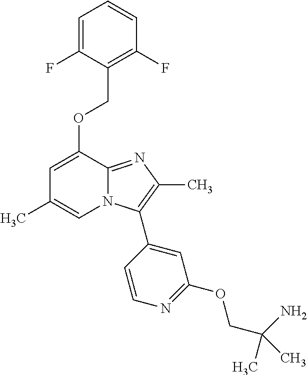 [SMILES: Cc1cc(OCc2c(F)cccc2F)c2nc(C)c(-c3ccnc(OCC(C)(C)N)c3)n2c1]

1 ml of a suspension of Raney nickel in water was added to a solution of 30 mg (0.062 mmol) of 8-(2,6-difluorobenzyl)oxy-2,6-dimethyl-3-[2-(2-methyl-2-nitropropoxy)pyridin-4-yl]imidazo[1,2-a]pyridine Example 20A in 10 ml of absolute ethanol. The content was hydrogenated at 1 bar at room temperature for 15 h and then filtered through Celite, and the mother liquor was concentrated to dryness under reduced pressure, which gave 7 mg (24% yield, purity 97%) of the target compound.

LC MS (Method 7): R^{t}=12.95 min; m/z=453.34 (M+H)⁺

¹H-NMR (500 MHz, DMSO-d₆): δ [ppm]=1.11 (s, 6H), 2.28 (s, 3H), 2.33 (s, 3H), 4.03 (s, 2H), 5.29 (s, 2H), 6.84 (s, 1H), 6.92 (s, 1H), 7.12 (d, 1H), 7.20-7.29 (m, 2H), 7.52-7.64 (m, 1H), 7.86 (s, 1H), 8.27 (d, 1H).

¹³C-NMR (125 MHz, DMSO-d₆): δ [ppm]=13.8, 18.0, 27.1, 49.3, 58.2, 75.4, 106.6, 109.5, 111.9, 112.2, 115.0, 116.6, 119.3, 122.0, 132.1, 137.8, 139.8, 141.6, 146.2, 147.7, 161.2, 164.5.

### Example 5

### (4E)-6-{8-[(2,6-Difluorobenzyl)oxy]-2,6-dimethylimidazo[1,2-a]pyridin-3-yl}-N-hydroxy-2,2-dimethyl-2,3-dihydropyridine-4(1H)-imine

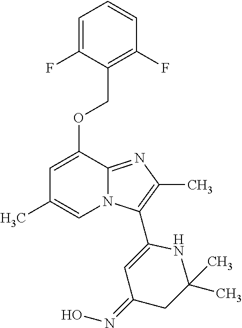

33.9 mg (0.49 mmol) of hydroxylamine hydrochloride were added to a solution of 150 mg (0.098 mmol, purity 28%) of 5-amino-1-{8-[(2,6-difluorobenzyl)oxy]-2,6-dimethylimidazo[1,2-a]pyridin-3-yl}-5-methylhexane-1,3-dione Example 23A in 5 ml of absolute ethanol. Under microwave irradiation, the resulting solution was heated at 120° C. for 20 min. The solvent was drawn off under reduced pressure and the residue was purified by preparative HPLC chromatography (Method 9), which gave 14 mg (34% yield, purity 98%) of the target compound.

LC MS (Method 8): Rₜ=7.60 min; m/z=427.19 (M+H)⁺

¹H-NMR (500 MHz, DMSO-d₆): δ [ppm]=1.23 (s, 6H), 2.26 (s, 2H), 2.28 (s, 6H), 5.27 (s, 2H), 5.37 (s, 1H), 6.38 (s, 1H), 6.77 (s, 1H), 7.23 (t, 2H), 7.59 (quint., 1H), 7.74 (s, 1H), 9.79 (s, 1H).

¹³C-NMR (125 MHz, DMSO-d₆): δ [ppm]=13.9, 18.3, 26.3, 40.4, 51.2, 58.2, 87.7, 106.1, 111.6, 112.2, 116.2, 119.2, 121.2, 132.5, 137.1, 138.5, 140.8, 146.3, 149.3, 161.1.

### Example 6

### 1-[(5-{8-[(2,6-Difluorobenzyl)oxy]-2,6-dimethylimidazo[1,2-a]pyridin-3-yl}-1H-1,2,4-triazol-3-yl)oxy]-2-methylpropan-2-amine

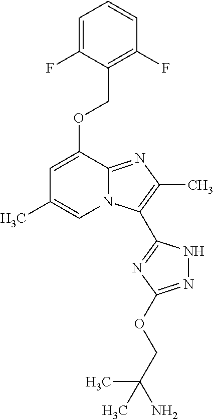

0.3 ml of Raney nickel (50% in water) was added to a solution of 66 mg (0.14 mmol) of 8-[(2,6-difluorobenzyl)oxy]-2,6-dimethyl-3-[3-(2-methyl-2-nitropropoxy)-1H-1,2,4-triazol-5-yl]imidazo[1,2-a]pyridine Example 25A in 3 ml of ethanol. At room temperature, the mixture was stirred under a hydrogen atmosphere (1 bar) overnight. The mixture was filtered through a layer of Celite which was washed with ethanol, dichloromethane and tetrahydrofuran. The combined filtrates were concentrated under reduced pressure and the residue was purified by flash chromatography using a silica gel cartridge (mobile phase: dichloromethane-2M ammonia in methanol 100:1 to 10:1), which gave 27 mg (42% yield, purity 98%) of the target compound.

LC-MS (Method 8): Rₜ=6.73 min; m/z=443.04 (M+H)⁺

¹H-NMR (500 MHz, DMSO-d₆): δ [ppm]=1.07 (s, 6H), 2.28 (s, 3H), 2.50 (s, 3H), 4.05 (s, 2H), 5.23 (s, 2H), 6.82 (s, 1H), 7.18 (t, 2H), 7.45-7.60 (m, 1H), 8.68 (s, 1H).

¹³C-NMR (126 MHz, DMSO-d6): δ [ppm]=15.0, 18.4, 25.9, 49.5, 58.2, 79.4, 106.7, 111.9, 112.2, 113.3, 117.5, 121.6, 132.1, 137.2, 142.3, 145.8, 150.3, 161.4, 164.3.

### Example 7

### 5-{8-[(2,6-Difluorobenzyl)oxy]-2,6-dimethylimidazo[1,2-a]pyridin-3-yl}pyridin-2(1H)-one

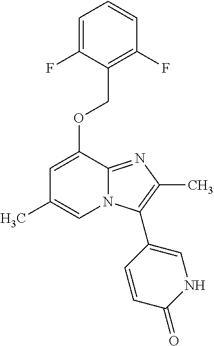

A mixture of 150 mg (0.41 mmol) of 3-bromo-8-[(2,6-difluorobenzyl)oxy]-2,6-dimethylimidazo[1,2-a]pyridine (Example 11A), 142 mg (1.03 mmol) of 6-hydroxy-3-pyridineboronic acid (CAS: 903899-13-8), 33 mg (0.041 mmol) of [1,1′-bis(diphenylphosphino)ferrocene]dichloropalladium(II) complex with dichloromethane and 0.61 ml (1.23 mmol) of 2N aqueous sodium carbonate solution in a mixture of ethanol (1 ml), toluene (2 ml) and water (1 ml) was stirred in a preheated oil bath at 90° C. for 4 h. The reaction mixture was cooled to room temperature and then concentrated under reduced pressure. The residue was purified by flash chromatography using a silica gel cartridge (mobile phase:dichloromethane-methanol 100:1 to 10:1), which gave 50 mg (26% yield, purity 81%) of the target compound.

LC-MS (Method 6): Rₜ=0.68 min; m/z=382 (M+H)⁺

¹H-NMR (300 MHz, DMSO-d₆): δ [ppm]=2.21 (s, 3H), 2.25 (s, 3H), 5.27 (s, 2H), 6.48 (dd, 1H), 6.76 (s, 1H), 7.23 (t, 2H), 7.45-7.64 (m, 4H), 11.84 (br.s, 1H).

### Example 8

### 4-{8-[(2,6-Difluorobenzyl)oxy]-2,6-dimethylimidazo[1,2-a]pyridin-3-yl}pyridin-2(1H)-one

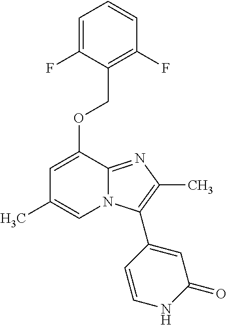 [SMILES: Cc1cc(OCc2c(F)cccc2F)c2nc(C)c(-c3cc[nH]c(=O)c3)n2c1]

A suspension of 115.5 mg (0.817 mmol) of (2-oxo-1,2-dihydropyridin-4-yl)boronic acid (CAS: 902148-83-8) in a mixture of 3 ml of absolute ethanol and 1 ml of toluene, followed by 47.4 mg (0.041 mmol) of tetrakis(triphenylphosphine)palladium(0) and a solution of 260.1 mg (1.225 mmol) of potassium phosphate in 1 ml of water were added to a solution of 150 mg (0.408 mmol) of 3-bromo-8-[(2,6-difluorobenzyl)oxy]-2,6-dimethylimidazo[1,2-a]pyridine Example 11A in 1 ml of absolute ethanol. The resulting mixture was stirred in a preheated oil bath at 90° C. for 4 h. After cooling to room temperature, the mixture was partitioned between ethyl acetate (30 ml) and water (10 ml). The phases were separated and the organic phase was concentrated under reduced pressure. The residue was purified by flash chromatography using a prepacked silica gel cartridge (mobile phase: dichloromethane-methanol 100:1 to 10:1), which gave 80 mg (50% yield, purity 97%) of the target compound.

LC-MS (Method 6): Rₜ=0.69 min; m/z=382.31 (M+H)⁺

¹H-NMR (300 MHz, DMSO-d₆): δ [ppm]=2.27 (s, 3H), 2.31 (s, 3H), 5.27 (s, 2H), 6.31 (dd, 1H), 6.38 (d, 1H), 6.81 (d, 1H), 7.22 (t, 2H), 7.49 (d, 1H), 7.59 (quint., 1H), 7.81 (t, 1H), 11.62 (br.s, 1H).

### B. ASSESSMENT OF PHARMACOLOGICAL EFFICACY

The following abbreviations are used:

ATP adenosine triphosphate
Brij35 polyoxyethylene(23) lauryl ether
BSA bovine serum albumin
DTT dithiothreitol
TEA triethanolamine

The pharmacological action of the compounds of the invention can be demonstrated in the following assays:

### B-1. Measurement of sGC Enzyme Activity by Means of PPi Detection

Soluble guanylyl cyclase (sGC) converts GTP to cGMP and pyrophosphate (PPi) when stimulated. PPi is detected with the aid of the method described in WO 2008/061626. The signal that arises in the assay increases as the reaction progresses and serves as a measure of the sGC enzyme activity. With the aid of a PPi reference curve, the enzyme can be characterized in a known manner, for example in terms of conversion rate, stimulability or Michaelis constant.

### Practice of the Test

To conduct the test, 29 μl of enzyme solution (0-10 nM soluble guanylyl cyclase (prepared according to Hönicka et al., Journal of Molecular Medicine 77(1999)14-23), in 50 mM TEA, 2 mM magnesium chloride, 0.1% BSA (fraction V), 0.005% Brij 35, pH 7.5) were initially charged in the microplate, and 1 μl of the stimulator solution (0-10 μM 3-morpholinosydnonimine, SIN-1, Merck in DMSO) was added. The microplate was incubated at RT for 10 min. Then 20 μl of detection mix (1.2 nM Firefly Luciferase (Photinus pyralis luciferase, Promega), 29 μM dehydroluciferin (prepared according to Bitler & McElroy, Arch. Biochem. Biophys. 72 (1957) 358), 122 μM luciferin (Promega), 153 μM ATP (Sigma) and 0.4 mM DTT (Sigma) in 50 mM TEA, 2 mM magnesium chloride, 0.1% BSA (fraction V), 0.005% Brij 35, pH 7.5) were added. The enzyme reaction was started by adding 20 μl of substrate solution (1.25 mM guanosine 5′-triphosphate (Sigma) in 50 mM TEA, 2 mM magnesium chloride, 0.1% BSA (fraction V), 0.005% Brij 35, pH 7.5) and analysed continuously in a luminometer.

### B-2. Effect on a Recombinant Guanylate Cyclase Reporter Cell Line

The cellular activity of the compounds according to the invention is determined using a recombinant guanylate cyclase reporter cell line, as described in F. Wunder et al., Anal. Biochem. 339, 104-112 (2005).

Representative MEC values (MEC=minimum effective concentration) for the compounds of the invention are shown in the table below (in some cases as mean values for individual determinations):

TABLE A

| Example no. | MEC [μM] |
|---|---|
| 1 | 1.0 |
| 2 | 0.3 |
| 3 | 1.0 |
| 4 | 0.03 |
| 5 | 0.03 |
| 6 | 3.0 |
| 8 | 1.0 |

### B-3. Vasorelaxant Effect In Vitro

Rabbits are stunned by a blow to the neck and exsanguinated. The aorta is removed, freed from adhering tissue and divided into rings of width 1.5 mm, which are placed individually under prestress into 5 ml organ baths with carbogen-sparged Krebs-Henseleit solution at 37° C. having the following composition (each mM): sodium chloride: 119; potassium chloride: 4.8; calcium chloride dihydrate: 1; magnesium sulphate heptahydrate: 1.4; potassium dihydrogenphosphate: 1.2; sodium bicarbonate: 25; glucose: 10. The contractile force is determined with Statham UC2 cells, amplified and digitalized using A/D transducers (DAS-1802 HC, Keithley Instruments Munich), and recorded in parallel on linear recorders. To obtain a contraction, phenylephrine is added to the bath cumulatively in increasing concentration. After several control cycles, the substance to be studied is added in increasing dosage each time in every further run, and the magnitude of the contraction is compared with the magnitude of the contraction attained in the last preceding run. This is used to calculate the concentration needed to reduce the magnitude of the control value by 50% (IC₅₀ value). The standard administration volume is 5 μl; the DMSO content in the bath solution corresponds to 0.1%.

### B-4. Blood Pressure Measurement on Anaesthetized Rats

Male Wistar rats having a body weight of 300-350 g are anaesthetized with thiopental (100 mg/kg i.p.). After tracheotomy, a catheter is introduced into the femoral artery to measure the blood pressure. The substances to be tested are administered as solutions, either orally by means of a gavage or intravenously via the femoral vein (Stasch et al. Br. J. Pharmacol. 2002; 135: 344-355).

### B-5. Radiotelemetry Measurement of Blood Pressure in Conscious, Spontaneously Hypertensive Rats

A commercially available telemetry system from DATA SCIENCES INTERNATIONAL DSI, USA, is employed for the blood pressure measurement on conscious rats described below.

The system consists of 3 main components:

implantable transmitters (Physiotel® telemetry transmitter)
receivers (Physiotel® receiver) which are linked via a multiplexer (DSI Data Exchange Matrix) to a
data acquisition computer.

The telemetry system makes it possible to continuously record blood pressure, heart rate and body motion of conscious animals in their usual habitat.

### Animal Material

The studies are conducted on adult female spontaneously hypertensive rats (SHR Okamoto) with a body weight of >200 g. SHR/NCrl from the Okamoto Kyoto School of Medicine, 1963, were a cross of male Wistar Kyoto rats having greatly elevated blood pressure and female rats having slightly elevated blood pressure, and were handed over at F13 to the U.S. National Institutes of Health.

After transmitter implantation, the experimental animals are housed singly in type 3 Makrolon cages. They have free access to standard feed and water.

The day/night rhythm in the experimental laboratory is changed by the room lighting at 6:00 am and at 7:00 pm.

### Transmitter Implantation

The TA11 PA-C40 telemetry transmitters used are surgically implanted under aseptic conditions in the experimental animals at least 14 days before the first experimental use. The animals instrumented in this way can be used repeatedly after the wound has healed and the implant has settled.

For the implantation, the fasted animals are anaesthetized with pentobarbital (Nembutal, Sanofi: 50 mg/kg i.p.) and shaved and disinfected over a large area of their abdomens. After the abdominal cavity has been opened along the linea alba, the liquid-filled measuring catheter of the system is inserted into the descending aorta in the cranial direction above the bifurcation and fixed with tissue glue (VetBonD™, 3M). The transmitter housing is fixed intraperitoneally to the abdominal wall muscle, and the wound is closed layer by layer.

An antibiotic (Tardomyocel COMP, Bayer, 1 ml/kg s.c.) is administered postoperatively for prophylaxis of infection.

### Substances and Solutions

Unless stated otherwise, the substances to be studied are administered orally by gavage to a group of animals in each case (n=6). In accordance with an administration volume of 5 ml/kg of body weight, the test substances are dissolved in suitable solvent mixtures or suspended in 0.5% tylose.

A solvent-treated group of animals is used as control.

### Experimental Outline

The telemetry measuring unit present is configured for 24 animals. Each experiment is recorded under an experiment number (Vyear month day).

Each of the instrumented rats living in the system is assigned a separate receiving antenna (1010 Receiver, DSI).

The implanted transmitters can be activated externally by means of an incorporated magnetic switch. They are switched to transmission in the run-up to the experiment. The signals emitted can be detected online by a data acquisition system (Dataquest™ A.R.T. for WINDOWS, DSI) and processed accordingly. The data are stored in each case in a file created for this purpose and bearing the experiment number.

In the standard procedure, the following are measured for 10-second periods in each case:

systolic blood pressure (SBP)
diastolic blood pressure (DBP)
mean arterial pressure (MAP)
heart rate (HR)
activity (ACT).

The acquisition of measurements is repeated under computer control at 5-minute intervals. The source data obtained as absolute values are corrected in the diagram with the currently measured barometric pressure (Ambient Pressure Reference Monitor; APR-1) and stored as individual data. Further technical details are given in the extensive documentation from the manufacturer company (DSI).

Unless indicated otherwise, the test substances are administered at 9:00 am on the day of the experiment. Following the administration, the parameters described above are measured over 24 hours.

### Evaluation

After the end of the experiment, the acquired individual data are sorted using the analysis software (DATAQUEST™ A.R.T.™ ANALYSIS). The blank value is assumed here to be the time 2 hours before administration, and so the selected data set encompasses the period from 7:00 am on the day of the experiment to 9:00 am on the following day.

The data are smoothed over a predefinable period by determination of the average (15-minute average) and transferred as a text file to a storage medium. The measured values presorted and compressed in this way are transferred to Excel templates and tabulated. For each day of the experiment, the data obtained are stored in a dedicated file bearing the number of the experiment. Results and test protocols are stored in files in paper form sorted by numbers.

### LITERATURE

Klaus Witte, Kai Hu, Johanna Swiatek, Claudia Müssig, Georg Ertl and Björn Lemmer: Experimental heart failure in rats: effects on cardiovascular circadian rhythms and on myocardial β-adrenergic signaling. Cardiovasc Res 47 (2): 203-405, 2000; Kozo Okamoto: Spontaneous hypertension in rats. Int Rev Exp Pathol 7: 227-270, 1969; Maarten van den Buuse: Circadian Rhythms of Blood Pressure, Heart Rate, and Locomotor Activity in Spontaneously Hypertensive Rats as Measured With Radio-Telemetry. Physiology & Behavior 55(4): 783-787, 1994.

### B-6. Determination of Pharmacokinetic Parameters Following Intravenous and Oral Administration

The pharmacokinetic parameters of the compounds according to the invention are determined in male CD-1 mice, male Wistar rats and female beagles. Intravenous administration in the case of mice and rats is carried out by means of a species-specific plasma/DMSO formulation, and in the case of dogs by means of a water/PEG400/ethanol formulation. In all species, oral administration of the dissolved substance is performed via gavage, based on a water/PEG400/ethanol formulation. The removal of blood from rats is simplified by inserting a silicone catheter into the right Vena jugularis externa prior to substance administration. The operation is carried out at least one day prior to the experiment with isofluran anaesthesia and administration of an analgesic (atropine/rimadyl (3/1) 0.1 ml s.c.). The blood is taken (generally more than 10 time points) within a time window including terminal time points of at least 24 to a maximum of 72 hours after substance administration. The blood is removed into heparinized tubes. The blood plasma is then obtained by centrifugation; if required, it is stored at −20° C. until further processing.

An internal standard (which may also be a chemically unrelated substance) is added to the samples of the compounds of the invention, calibration samples and qualifiers, and there follows protein precipitation by means of acetonitrile in excess. Addition of a buffer solution matched to the LC conditions, and subsequent vortexing, is followed by centrifugation at 1000 g. The supernatant is analysed by LC-MS/MS using C18 reversed-phase columns and variable mobile phase mixtures. The substances are quantified via the peak heights or areas from extracted ion chromatograms of specific selected ion monitoring experiments.

The plasma concentration/time plots determined are used to calculate the pharmacokinetic parameters such as AUC, Cₘₐₓ, t_{1/2} (terminal half-life), F (bioavailability), MRT (mean residence time) and CL (clearance), by means of a validated pharmacokinetic calculation program.

Since the substance quantification is performed in plasma, it is necessary to determine the blood/plasma distribution of the substance in order to be able to adjust the pharmacokinetic parameters correspondingly. For this purpose, a defined amount of substance is incubated in heparinized whole blood of the species in question in a rocking roller mixer for 20 min. After centrifugation at 1000 g, the plasma concentration is measured (by means of LC-MS/MS; see above) and determined by calculating the ratio of the C_{blood}/Cₚₗₐₛₘₐ value.

### B-7. Metabolic Study

To determine the metabolic profile of the inventive compounds, they are incubated with recombinant human cytochrome P450 (CYP) enzymes, liver microsomes or primary fresh hepatocytes from various animal species (e.g. rats, dogs), and also of human origin, in order to obtain and to compare information about a very substantially complete hepatic phase I and phase II metabolism, and about the enzymes involved in the metabolism.

The compounds of the invention were incubated with a concentration of about 0.1-10 μM. To this end, stock solutions of the compounds of the invention having a concentration of 0.01-1 mM in acetonitrile were prepared, and then pipetted with a 1:100 dilution into the incubation mixture. Liver microsomes and recombinant enzymes were incubated at 37° C. in 50 mM potassium phosphate buffer pH 7.4 with and without NADPH-generating system consisting of 1 mM NADP⁺, 10 mM glucose-6-phosphate and 1 unit glucose-6-phosphate dehydrogenase. Primary hepatocytes were incubated in suspension in Williams E medium, likewise at 37° C. After an incubation time of 0-4 h, the incubation mixtures were stopped with acetonitrile (final concentration about 30%) and the protein was centrifuged off at about 15 000×g. The samples thus stopped were either analysed directly or stored at −20° C. until analysis.

The analysis is carried out by high-performance liquid chromatography with ultraviolet and mass spectrometry detection (HPLC-UV-MS/MS). To this end, the supernatants of the incubation samples are chromatographed with suitable C18 reversed-phase columns and variable eluent mixtures of acetonitrile and 10 mM aqueous ammonium formate solution or 0.05% formic acid. The UV chromatograms in conjunction with mass spectrometry data serve for identification, structural elucidation and quantitative estimation of the metabolites, and for quantitative metabolic reduction of the compound of the invention in the incubation mixtures.

### B-8. Caco-2 Permeability Test

The permeability of a test substance was determined with the aid of the Caco-2 cell line, an established in vitro model for permeability prediction at the gastrointestinal barrier (Artursson, P. and Karlsson, J. (1991). Correlation between oral drug absorption in humans and apparent drug permeability coefficients in human intestinal epithelial (Caco-2) cells. Biochem. Biophys. 175 (3), 880-885). The Caco-2 cells (ACC No. 169, DSMZ, Deutsche Sammlung von Mikroorganismen und Zellkulturen, Braunschweig, Germany) were sown in 24-well plates having an insert and cultivated for 14 to 16 days. For the permeability studies, the test substance was dissolved in DMSO and diluted to the final test concentration with transport buffer (Hanks Buffered Salt Solution, Gibco/Invitrogen, with 19.9 mM glucose and 9.8 mM HEPES). In order to determine the apical to basolateral permeability (PₐₚₚA-B) of the test substance, the solution comprising the test substance was applied to the apical side of the Caco-2 cell monolayer, and transport buffer to the basolateral side. In order to determine the basolateral to apical permeability (PₐₚₚB-A) of the test substance, the solution comprising the test substance was applied to the basolateral side of the Caco-2 cell monolayer, and transport buffer to the apical side. At the start of the experiment, samples were taken from the respective donor compartment in order to ensure the mass balance. After an incubation time of two hours at 37° C., samples were taken from the two compartments. The samples were analysed by means of LC-MS/MS and the apparent permeability coefficients (Pₐₚₚ) were calculated. For each cell monolayer, the permeability of Lucifer Yellow was determined to ensure cell layer integrity. In each test run, the permeability of atenolol (marker for low permeability) and sulfasalazine (marker for active excretion) was also determined as quality control.

B-9. hERG Potassium Current Assay

The hERG (human ether-a-go-go related gene) potassium current makes a significant contribution to the repolarization of the human cardiac action potential (Scheel et al., 2011). Inhibition of this current by pharmaceuticals can in rare cases cause potentially lethal cardiac arrhythmias, and is therefore studied at an early stage during drug development.

The functional hERG assay used here is based on a recombinant HEK293 cell line which stably expresses the KCNH2(HERG) gene (Zhou et al., 1998). These cells are studied by means of the “whole-cell voltage-clamp” technique (Hamill et al., 1981) in an automated system (Patchliner™; Nanion, Munich, Germany), which controls the membrane voltage and measures the hERG potassium current at room temperature. The PatchControlHT™ software (Nanion) controls the Patchliner system, data capture and data analysis. The voltage is controlled by 2 EPC-10 quadro amplifiers controlled by the PatchMasterPro™ software (both: HEKA Elektronik, Lambrecht, Germany). NPC-16 chips with moderate resistance (˜2 MΩ; Nanion) serve as the planar substrate for the voltage clamp experiments.

NPC-16 chips are filled with intra- and extracellular solution (cf. Himmel, 2007) and with cell suspension. After forming a gigaohm seal and establishing whole-cell mode (including several automated quality control steps), the cell membrane is clamped at the −80 mV holding potential. The subsequent voltage clamp protocol changes the command voltage to +20 mV (for 1000 ms), −120 mV (for 500 ms), and back to the −80 mV holding potential; this is repeated every 12 s. After an initial stabilization phase (about 5-6 minutes), test substance solution is introduced by pipette in rising concentrations (e.g. 0.1, 1, and 10 μmol/l) (exposure about 5-6 minutes per concentration), followed by several washing steps.

The amplitude of the inward “tail” current which is generated by a change in potential from +20 mV to −120 mV serves to quantify the hERG potassium current, and is described as a function of time (IgorPro™ Software). The current amplitude at the end of various time intervals (for example stabilization phase before test substance, first/second/third concentration of test substance) serves to establish a concentration/effect curve, from which the half-maximum inhibiting concentration IC₅₀ of the test substance is calculated.
Hamill O P, Marty A, Neher E, Sakmann B, Sigworth F J. Improved patch-clamp techniques for high-resolution current recording from cells and cell-free membrane patches. Pfluegers Arch 1981; 391:85-100.
Himmel H M. Suitability of commonly used excipients for electrophysiological in-vitro safety pharmacology assessment of effects on hERG potassium current and on rabbit Purkinje fiber action potential. J Pharmacol Toxicol Methods 2007; 56:145-158.
Scheel O, Himmel H, Rascher-Eggstein G, Knott T. Introduction of a modular automated voltage-clamp platform and its correlation with manual human ether-a-go-go related gene voltage-clamp data. Assay Drug Dev Technol 2011; 9:600-607.
Zhou Z F, Gong Q, Ye B, Fan Z, Makielski J C, Robertson G A, January C T. Properties of hERG channels stably expressed in HEK293 cells studied at physiological temperature. Biophys J 1998; 74:230-241.

### C. WORKING EXAMPLES OF PHARMACEUTICAL COMPOSITIONS

The compounds of the invention can be converted to pharmaceutical preparations as follows:

### Tablet:

### Composition:

100 mg of the compound of the invention, 50 mg of lactose (monohydrate), 50 mg of corn starch (native), 10 mg of polyvinylpyrrolidone (PVP 25) (BASF, Ludwigshafen, Germany) and 2 mg of magnesium stearate.

Tablet weight 212 mg. Diameter 8 mm, radius of curvature 12 mm.

### Production:

The mixture of compound of the invention, lactose and starch is granulated with a 5% solution (w/w) of the PVP in water. The granules are dried and then mixed with the magnesium stearate for 5 minutes. This mixture is compressed using a conventional tabletting press (see above for format of the tablet). The guide value used for the pressing is a pressing force of 15 kN.

### Suspension for Oral Administration:

### Composition:

1000 mg of the compound of the invention, 1000 mg of ethanol (96%), 400 mg of Rhodigel® (xanthan gum from FMC, Pennsylvania, USA) and 99 g of water.

10 ml of oral suspension correspond to a single dose of 100 mg of the compound of the invention.

### Production:

The Rhodigel is suspended in ethanol; the compound of the invention is added to the suspension. The water is added while stirring. The mixture is stirred for about 6 h until the swelling of the Rhodigel is complete.

### Solution for Oral Administration:

### Composition:

500 mg of the compound of the invention, 2.5 g of polysorbate and 97 g of polyethylene glycol 400. 20 g of oral solution correspond to a single dose of 100 mg of the compound of the invention.

### Production:

The compound of the invention is suspended in the mixture of polyethylene glycol and polysorbate with stirring. The stirring operation is continued until dissolution of the compound of the invention is complete.

i.v. Solution:

The compound of the invention is dissolved in a concentration below the saturation solubility in a physiologically acceptable solvent (e.g. isotonic saline solution, glucose solution 5% and/or PEG 400 solution 30%). The resulting solution is subjected to sterile filtration and dispensed into sterile and pyrogen-free injection vessels.

1. Compound of the formula (I) 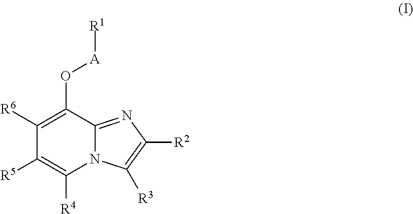
in which
A represents CH₂, CD₂ or CH(CH₃),
R¹ represents (C₃-C₇)-cycloalkyl, phenyl or pyridyl,
where (C₃-C₇)-cycloalkyl may be substituted by 1 to 4 substituents independently of one another selected from the group consisting of fluorine, trifluoromethyl and (C₁-C₄)-alkyl,
where phenyl is substituted by 1 to 4 substituents independently of one another selected from the group consisting of halogen, cyano, monofluoromethyl, difluoromethyl, trifluoromethyl, (C₁-C₄)-alkyl, (C₁-C₄)-alkoxy and difluoromethoxy
and
where pyridyl is substituted by 1 or 2 substituents independently of one another selected from the group consisting of halogen, cyano and (C₁-C₄)-alkyl,
R² represents (C₁-C₄)-alkyl, cyclopropyl, cyclobutyl, monofluoromethyl, difluoromethyl or trifluoromethyl,
R³ represents a group of the formula 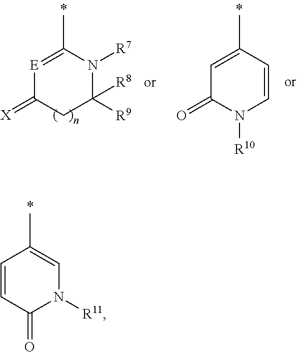
where
* represents the point of attachment to the imidazo[1,2-a]pyridine ring,
E represents carbon or nitrogen,
n represents 0, 1 or 2,
X represents oxygen or nitrogen,
in which nitrogen may be substituted by hydrogen or hydroxy,
R⁷ represents hydrogen or (C₁-C₆)-alkyl,
in which (C₁-C₆)-alkyl may be substituted by amino or hydroxy,
and
in which (C₁-C₆)-alkyl may be substituted up to five times by fluorine,
R⁸ represents hydrogen or (C₁-C₄)-alkyl,
in which (C₁-C₄)-alkyl may be substituted up to five times by fluorine,
R⁹ represents hydrogen or (C₁-C₄)-alkyl,
in which (C₁-C₄)-alkyl may be substituted up to five times by fluorine,
or
R⁸ and R⁹ together with the carbon atom to which they are bonded form a 3- to 7-membered carbocycle or a 4- to 7-membered heterocycle,
in which the 3- to 7-membered carbocycle and the 4- to 7-membered heterocycle may in turn be substituted by 1 or 2 substituents independently of one another selected from the group consisting of fluorine and (C₁-C₄)-alkyl,
R¹⁰ represents hydrogen or (C₁-C₈)-alkyl,
in which (C₁-C₈)-alkyl may be substituted by amino or hydroxy,
and
in which (C₁-C₈)-alkyl may be substituted up to five times by fluorine,
R¹¹ represents hydrogen or (C₁-C₈)-alkyl,
in which (C₁-C₈)-alkyl may be substituted by amino or hydroxy,
and
in which (C₁-C₈)-alkyl may be substituted up to five times by fluorine,
or
represents 5- to 10-membered heteroaryl,
where 5- to 10-membered heteroaryl is substituted by (C₁-C₈)-alkoxy,
in which (C₁-C₈)-alkoxy is substituted by amino,
and
in which (C₁-C₈)-alkoxy may be substituted up to five times by fluorine,
and
where 5- to 10-membered heteroaryl may be substituted by 1 or 2 substituents independently of one another selected from the group consisting of halogen, cyano, trifluoromethyl, difluoromethyl and (C₁-C₆)-alkyl,
R⁴ represents hydrogen,
R⁵ represents hydrogen, halogen, cyano, (C₁-C₄)-alkyl, (C₂-C₄)-alkynyl, (C₁-C₄)-alkoxy, (C₃-C₅)-cycloalkyl, difluoromethoxy, difluoromethyl, trifluoromethyl, 4- to 7-membered heterocyclyl or 5- or 6-membered heteroaryl,
R⁶ represents hydrogen or halogen,
and the N-oxides, salts, solvates, salts of the N-oxides and solvates of the N-oxides and salts thereof.

2. Compound of the formula (I) according to claim 1 in which
A represents CH₂ or CD₂,
R¹ represents cyclohexyl, phenyl or pyridyl,
where phenyl is substituted by 1 to 4 substituents independently of one another selected from the group consisting of fluorine, bromine, chlorine, cyano and methyl,
and
where pyridyl is substituted by 1 or 2 substituents independently of one another selected from the group consisting of fluorine, cyano and methyl,
R² represents (C₁-C₄)-alkyl, cyclopropyl or trifluoromethyl,
R³ represents a group of the formula 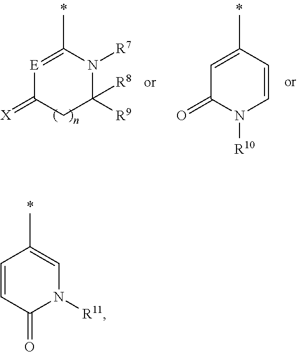
where
* represents the point of attachment to the imidazo[1,2-a]pyridine ring,
E represents carbon or nitrogen,
n represents 0 or 1,
X represents oxygen or nitrogen,
in which nitrogen may be substituted by hydrogen or hydroxy,
R⁷ represents hydrogen or (C₁-C₆)-alkyl,
in which (C₁-C₆)-alkyl may be substituted by amino or hydroxy,
and
in which (C₁-C₆)-alkyl may be substituted up to five times by fluorine,
R⁸ represents hydrogen or (C₁-C₄-alkyl,
in which (C₁-C₄)-alkyl may be substituted up to five times by fluorine,
R⁹ represents hydrogen or (C₁-C₄)-alkyl,
in which (C₁-C₄)-alkyl may be substituted up to five times by fluorine,
or
R⁸ and R⁹ together with the carbon atom to which they are bonded form a 3- to 7-membered carbocycle,
in which the 3- to 7-membered carbocycle may be substituted by 1 or 2 substituents independently of one another selected from the group consisting of fluorine and methyl,
R¹⁰ represents hydrogen or (C₁-C₈)-alkyl,
in which (C₁-C₈)-alkyl may be substituted by amino or hydroxy,
and
in which (C₁-C₈)-alkyl may be substituted up to five times by fluorine,
R¹¹ represents hydrogen or (C₁-C₈)-alkyl,
in which (C₁-C₈)-alkyl may be substituted by amino or hydroxy,
and
in which (C₁-C₈)-alkyl may be substituted up to five times by fluorine,
or
represent a group of the formula 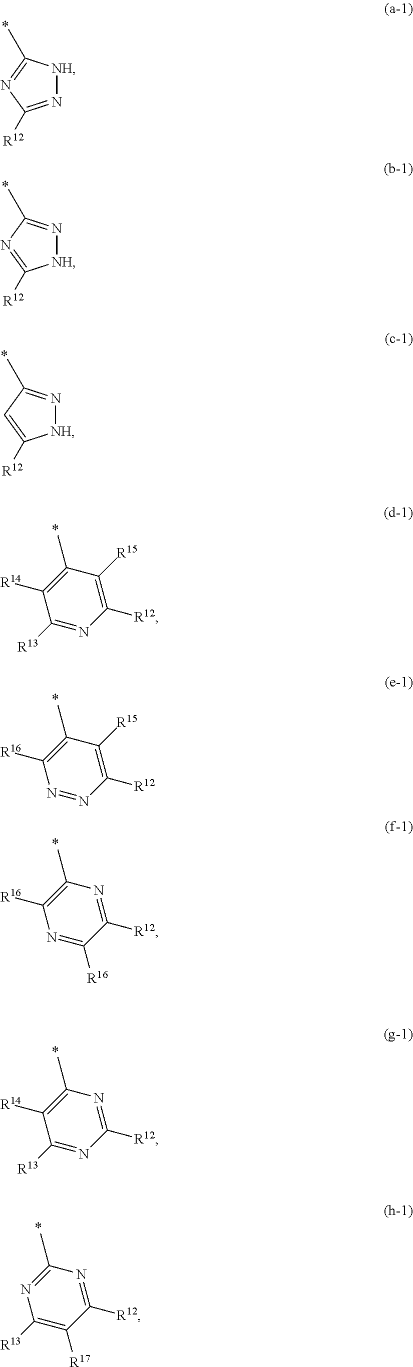
where
R¹² represents (C₁-C₈)-alkoxy,
in which (C₁-C₈)-alkoxy is substituted by amino,
and
in which (C₁-C₈)-alkoxy may be substituted up to five times by fluorine,
and
R¹³ represents hydrogen, cyano, trifluoromethyl, difluoromethyl or methyl,
R¹⁴ represents hydrogen, fluorine, chlorine, cyano, trifluoromethyl, difluoromethyl or methyl,
R¹⁵ represents hydrogen, fluorine, chlorine, cyano, trifluoromethyl, difluoromethyl or methyl,
R¹⁶ represents hydrogen, cyano, trifluoromethyl, difluoromethyl or methyl,
R¹⁷ represents hydrogen, fluorine, chlorine, cyano, trifluoromethyl, difluoromethyl or methyl,
R⁴ represents hydrogen,
R⁵ represents hydrogen, chlorine, cyano, methyl, methoxy or cyclopropyl,
R⁶ represents hydrogen or fluorine,
and the N-oxides, salts, solvates, salts of the N-oxides and solvates of the N-oxides and salts thereof.

3. Compound of the formula (I) according to claim 1 in which
A represents CH₂,
R¹ represents a phenyl group of the formula 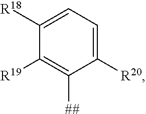
where
## represents the point of attachment to A,
and
R¹⁸, R¹⁹ and R²⁰ independently of one another represent hydrogen or fluorine,
with the proviso that at least two of the radicals R¹⁸, R¹⁹, R²⁰ are different from hydrogen,
R² represents methyl,
R³ represents a group of the formula 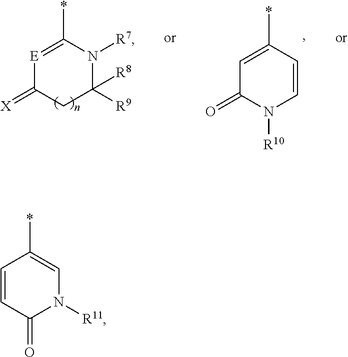
where
* represents the point of attachment to the imidazo[1,2-a]pyridine ring,
E represents carbon or nitrogen,
n represents 1,
X represents oxygen or nitrogen,
in which nitrogen may be substituted by hydrogen or hydroxy,
R⁷ represents hydrogen,
R⁸ represents hydrogen, methyl or ethyl,
in which methyl may be substituted up to three times by fluorine,
and
in which ethyl may be substituted up to five times by fluorine,
R⁹ represents hydrogen, methyl or ethyl,
in which methyl may be substituted up to three times by fluorine,
and
in which ethyl may be substituted up to five times by fluorine,
or
R⁸ and R⁹ together with the carbon atom to which they are bonded form a 3- to 6-membered carbocycle,
R¹⁰ represents hydrogen or (C₁-C₈)-alkyl,
in which (C₁-C₈)-alkyl is substituted by amino,
and
in which (C₁-C₈)-alkyl may be substituted up to five times by fluorine,
R¹¹ represents hydrogen or (C₁-C₈)-alkyl,
in which (C₁-C₈)-alkyl is substituted by amino,
and
in which (C₁-C₈)-alkyl may be substituted up to five times by fluorine,
or
represent a group of the formula 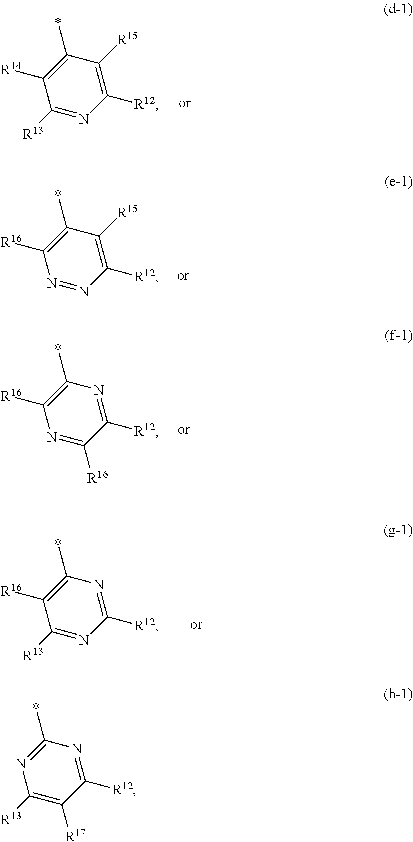
where
R¹² represents (C₁-C₈)-alkoxy,
in which (C₁-C₈)-alkoxy is substituted by amino,
and
in which (C₁-C₈)-alkoxy may be substituted up to five times by fluorine,
R¹³ represents hydrogen or methyl,
R¹⁴ represents hydrogen, fluorine, chlorine or methyl,
R¹⁵ represents hydrogen, fluorine, chlorine or methyl,
R¹⁶ represents hydrogen or methyl,
and
R¹⁷ represents hydrogen, fluorine, chlorine or methyl,
R⁴ represents hydrogen,
R⁵ represents hydrogen, chlorine or methyl,
R⁶ represents hydrogen,
and the N-oxides, salts, solvates, salts of the N-oxides and solvates of the N-oxides and salts thereof.

4. Compound of the formula (I) according to claim 1, in which
A represents CH₂,
R¹ represents a phenyl group of the formula 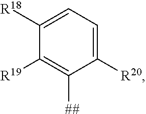
where
## represents the point of attachment to A,
and
R¹⁸, R¹⁹ and R²⁰ independently of one another represent hydrogen or fluorine,
with the proviso that at least two of the radicals R¹⁸, R¹⁹, R²⁰ are different from hydrogen,
R² represents methyl,
R³ represents a group of the formula 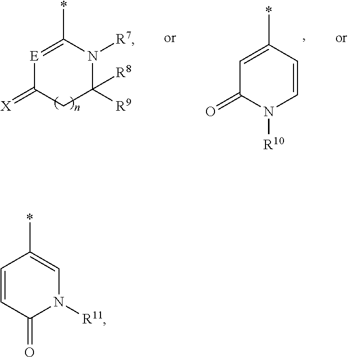
where
* represents the point of attachment to the imidazo[1,2-a]pyridine ring,
E represents carbon or nitrogen,
n represents 1,
X represents oxygen or nitrogen,
in which nitrogen may be substituted by hydrogen or hydroxy,
R⁷ represents hydrogen,
R⁸ represents hydrogen, methyl or ethyl,
in which methyl may be substituted up to three times by fluorine,
and
in which ethyl may be substituted up to five times by fluorine,
R⁹ represents hydrogen, methyl or ethyl,
in which methyl may be substituted up to three times by fluorine,
and
in which ethyl may be substituted up to five times by fluorine
or
R⁸ and R⁹ together with the carbon atom to which they are attached form a cyclopropyl ring,
R¹⁰ represents hydrogen or (C₁-C₆)-alkyl,
in which (C₁-C₆)-alkyl is substituted by amino,
and
in which (C₁-C₆)-alkyl may be substituted up to five times by fluorine,
R¹¹ represents hydrogen or (C₁-C₆)-alkyl,
in which (C₁-C₆)-alkyl is substituted by amino,
and
in which (C₁-C₆)-alkyl may be substituted up to five times by fluorine,
or
represent a group of the formula 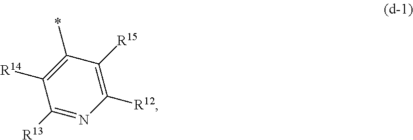
where
R¹² represents (C₁-C₆)-alkoxy,
in which (C₁-C₆)-alkoxy is substituted by amino,
and
in which (C₁-C₆)-alkoxy may be substituted up to five times by fluorine,
R¹³ represents hydrogen,
R¹⁴ represents hydrogen or fluorine,
R¹⁵ represents hydrogen or fluorine,
R⁴ represents hydrogen,
R⁵ represents hydrogen, chlorine or methyl,
R⁶ represents hydrogen,
and the N-oxides, salts, solvates, salts of the N-oxides and solvates of the N-oxides and salts thereof.

5. Process for preparing the compound of claim 1, comprising:
reacting a compound of the formula (II) 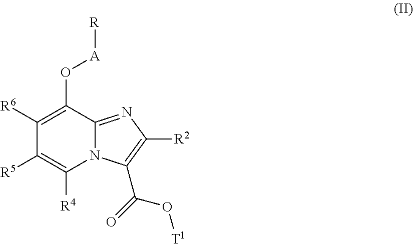
in which A, R¹, R², R⁴, R⁵ and R⁶ are each as defined in claim 1 and
T¹ represents (C₁-C₄)-alkyl or benzyl,
in an inert solvent in the presence of a suitable base or acid to give a carboxylic acid of the formula (III) 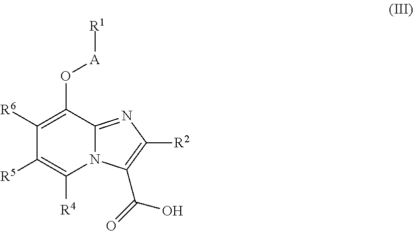
in which A, R¹, R², R⁴, R⁵ and R⁶ each have the meanings given above,
and subsequently reacting the carboxylic acid of the formula (III) in the presence of a suitable acid to give an imidazo[1,2-a]pyridine of the formula (IV) 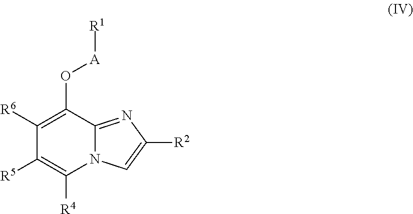
in which A, R¹, R², R⁴, R⁵ and R⁶ each have the meanings given above,
and converting the imidazo[1,2-a]pyridine of the formula (IV) with a halogen equivalent into a compound of the formula (V) 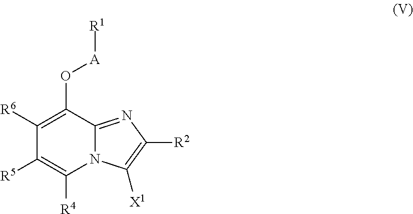
in which A, R¹, R², R⁴, R⁵ and R⁶ are each as defined above and
X¹ represents chlorine, bromine or iodine,
and subsequently reacting the compound of the formula (V) in an inert solvent, in the presence of a suitable transition metal catalyst, with a compound of the formula (VI) 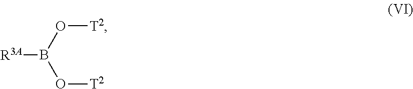
in which
R^{3A} has the meanings given above for R³
and
T² represents hydrogen or (C₁-C₄)-alkyl, or the two T² radicals together form a —C(CH₃)₂—C(CH₃)₂— bridge,
to give a compound of the formula (I-A) 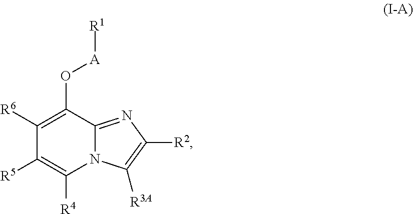
and, if R^{3A} represents 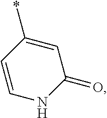 [SMILES: *c1cc[nH]c(=O)c1]
reacting the compound of the formula (I-A) in an inert solvent in the presence of a suitable base with a compound of the formula (VIII)
R^{10A}—X² (VIII)
in which
X² represents a suitable leaving group, in particular chlorine, bromine, iodine, mesylate, triflate or tosylate,
and
R^{10A} represents (C₁-C₈)-alkyl,
in which (C₁-C₈)-alkyl is substituted by nitro,
and
in which (C₁-C₈)-alkyl may be substituted up to five times by fluorine,
to give compounds of the formula (VII-A) or (VII-B) 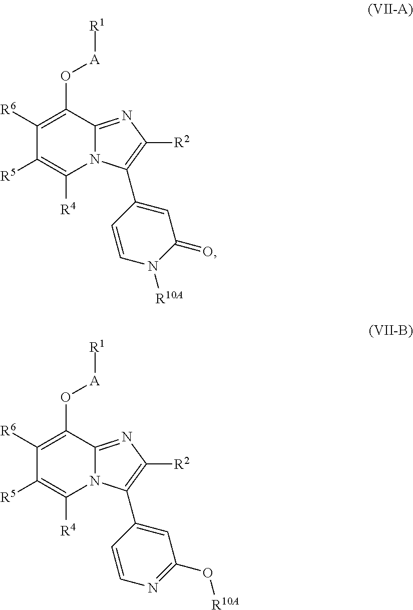
in which A, R¹, R², R⁴, R⁵ and R⁶ each have the meanings given above
and
R^{10A} represents (C₁-C₈)-alkyl,
in which (C₁-C₈)-alkyl is substituted by nitro,
and
in which (C₁-C₈)-alkyl may be substituted up to five times by fluorine,
and converting the nitro compounds in an inert solvent in the presence of Raney nickel or palladium/carbon in a hydrogen atmosphere into compounds of the formula (I-B and I-C) 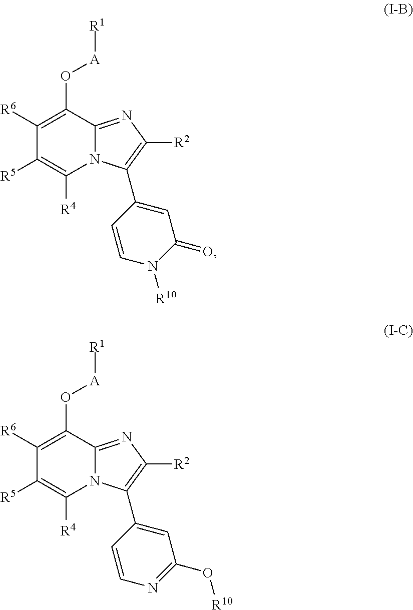
in which A, R¹, R², R⁴, R⁵, R⁶ and R¹⁰ each have the meanings given above,
and detaching any protective groups present, and the resulting compounds of the formula (I) are optionally converted with the appropriate (i) solvents and/or (ii) acids or bases to the solvates, salts and/or solvates of the salts thereof.

6. (canceled)

7. (canceled)

8. Medicament comprising a compound of the formula (I) as defined in claim 1 in combination with an inert, non-toxic, pharmaceutically suitable auxiliary.

9. Medicament comprising a compound of the formula (I) as defined in claim 1 in combination with a further active compound selected from the group consisting of organic nitrates, NO donors, cGMP-PDE inhibitors, antithrombotic agents, hypotensive agents and lipid metabolism modifiers.

10. (canceled)

11. Method for the treatment and/or prophylaxis of heart failure, angina pectoris, hypertension, pulmonary hypertension, ischaemias, vascular disorders, renal insufficiency, thromboembolic disorders and arteriosclerosis comprising administering an effective amount of at least one compound of the formula (I) as defined in claim 1 to a human or animal in need thereof.

12. Method for treatment and/or prophylaxis of heart failure, angina pectoris, hypertension, pulmonary hypertension, ischaemias, vascular disorders, renal insufficiency, thromboembolic disorders and arteriosclerosis comprising administering an effective amount of the medicament of claim 8 to a human or animal in need thereof.

13. Method for treatment and/or prophylaxis of heart failure, angina pectoris, hypertension, pulmonary hypertension, ischaemias, vascular disorders, renal insufficiency, thromboembolic disorders and arteriosclerosis comprising administering an effective amount of the medicament of claim 9 to a human or animal in need thereof.
